(19) United States
(12) Patent Application Publication
(10) Pub. No.: US 2025/0237648 A1
(43) Pub. Date: Jul. 24, 2025

(54) BACTERIAL BIOSENSORS FOR DIAGNOSING AND TREATING INFLAMMATION

(71) Applicant: Baylor College of Medicine, Houston, TX (US)

(72) Inventors: Robert Britton, Houston, TX (US); Duolong Zhu, Houston, TX (US)

(73) Assignee: Baylor College of Medicine, Houston, TX (US)

(21) Appl. No.: 18/855,810

(22) PCT Filed: Apr. 12, 2023
(86) PCT No.: PCT/US2023/065647

§ 371 Date: Oct. 10, 2024

Related U.S. Application Data
(60) Provisional application No. 63/330,597, filed on Apr. 13, 2022.

(51) Int. Cl. G01N 33/569; A61K 38/20; A61P 1/00

(52) U.S. Cl. CPC G01N 33/56911; A61K 38/2066; A61P 1/00; G01N 2333/245; G01N 2333/33; G01N 2333/5428

(57) ABSTRACT

Embodiments of the disclosure relate to methods, compositions, and symptoms for detecting the imminent onset of a symptom of a gut inflammation medical condition and/or treatment thereof. The disclosure concerns microbial biosensors that detect a marker in the gut that is predictive of onset of at least one symptom of gut inflammation, such as with inflammatory bowel disease (IBD), for example, and such a sensor may include a promoter sensitive to the marker that is linked to expression of a detectable readout, such as in the feces of the individual. In various embodiments, the sensor includes a mechanism by which the biosensor is permanently activated to sense inflammation for future detection in the stool.

This application claims priority to U.S. Provisional Patent Application Ser. No. 63/330,597, filed Apr. 13, 2022, which is incorporated by reference herein in its entirety.

### TECHNICAL FIELD

Embodiments of the disclosure include at least the fields of cell biology, molecular biology, bacteriology, gastroenterology, inflammation, diagnosis, and medicine.

### BACKGROUND

The inflammatory bowel diseases (IBD), which include Crohn's Disease (CD) and ulcerative colitis (UC), are chronic gastrointestinal (GI) disorders often associated with periodic symptomatic relapses. These episodes are caused by a hyperactive inflammatory response and the subsequent release of a cascade of damaging mediators [1]. Such flares are unpredictable in nature, and have a high probability of occurring on a yearly basis for IBD patients [2]. Compounding the difficulties associated with the erratic and disruptive nature of IBD symptomology are the current disease detection and maintenance options. Many of these methods, including endoscopy or magnetic resonance imaging (MRI), are invasive and costly. As a result of these negative aspects, they are not a realistic option for frequent diagnostic evaluations of IBD relapse. In recent years, researchers have identified calprotectin as a validated fecal marker for IBD, giving patients a cheaper option for disease monitoring [3]. Calprotectin is a neutrophil-source antimicrobial peptide that impinges upon bacterial growth through free metal chelation by sequestering zinc, manganese, and iron [4-6]. Fecal calprotectin assays identify concentrations above 100 μg/mL as being positive for GI inflammation, but also contain a range of borderline levels between 50 and 100 μg/mL calprotectin that require retesting due to lower predictive value [7,8]. Herein lies a major issue, as compliance on retests can be low [9]. Thus, despite the reduced invasion and cost to the patient, fecal calprotectin detection can still miss the onset of a symptomatic flare due to missed retests on borderline results, as well as infrequent administration (3× per year) and primarily being used reactively to symptom onset instead of pre-emptively.

A need exists for an IBD-monitoring method that is rapid and allows for more frequent observation and oversight by clinician and patient, and the present disclosure provides a solution for that long-felt need.

### BRIEF SUMMARY

Embodiments of the disclosure provide systems, methods, and compositions related to monitoring of a medical condition, including a gastrointestinal and/or inflammatory medical condition. In particular embodiments, the monitoring concerns a biological forewarning or confirmatory system for onset of one or more symptoms for patients with, or at risk for, a gastrointestinal inflammatory condition, such as an inflammatory bowel disease (IBD) or an infection. In various embodiments, the systems, methods, and compositions allow for detection of any event in which neutrophils are involved. In particular embodiments, the system allows the patient to be notified before the onset of one or more particular symptoms of a gastrointestinal inflammatory condition or in some cases in early stages of the gastrointestinal inflammatory condition. In a specific embodiment, the system allows the patient to become aware of imminent onset of one or more symptoms without initial oversight by a medical practitioner. In at least certain cases, the system is sufficiently sensitive to detect biological signals of the medical condition in vivo before a symptom of the medical condition detectably manifests, including before one or more unpleasant or deleterious symptoms occur. In specific embodiments, the individual is in remission for a gastrointestinal inflammatory condition and desires to monitor their health. In various embodiments, the systems, methods, and compositions of the disclosure allow for an individual to monitor for the gastrointestinal inflammatory condition in a private setting, such as removed from a medical facility, and in cases wherein there is no direct or real-time oversight from a medical practitioner. The monitoring may be random or periodic, including daily, weekly, monthly, and so on. Any method encompassed herein avoids the need for handling of feces by the individual, in specific embodiments. Instead, the monitoring occurs by observation, in various embodiments, and may or may not require one or more instruments other than visual observation for detection.

The systems, methods, and compositions of the present disclosure may be utilized in connection with any medical conditions that involve chronic inflammation of any kind. In some cases, the inflammation is of the digestive tract, a hallmark of IBD. Examples of particular IBDs include Crohn's disease and ulcerative colitis. In particular embodiments, the system detects a biological marker associated with a gastrointestinal symptom and the detection manifests in the feces of the individual. In specific embodiments, the detection of the biological marker occurs before manifestation of one or more symptoms from the inflammation occur. In at least some cases, the detection involves detection of a microbial biosensor that is sensitive to a fecal marker associated with a gastrointestinal inflammatory condition, including IBD. In specific embodiments, the marker is a marker of neutrophil activity, including activity associated with any inflammatory event in which neutrophils play a direct or indirect role, as they are the source of a marker, calprotectin (or the zinc sequestration caused by calprotectin). In particular embodiments, the microbial biosensor comprises bacteria engineered to detect the marker associated with gastrointestinal inflammatory condition such that the bacteria emit and/or secrete a detectable signal representative of the marker and, indirectly, the gastrointestinal inflammatory condition. In particular embodiments, the bacteria are engineered to upregulate expression of a detectable gene product in the presence of calprotectin utilizing calprotectin-responsive sequences in the regulatory sequences operably linked to the detectable gene product.

Embodiments of the disclosure include methods of determining a need for therapy for intestinal inflammation or cancer in an individual, comprising the steps of providing to the individual a population of non-pathogenic bacteria comprising at least one engineered polynucleotide, said polynucleotide comprising one or more direct calprotectin-responsive sequences or indirect calprotectin-responsive sequences operably linked to expression of a detectable readout product; and examining the feces of said individual for the detectable readout product. The calprotectin-responsive sequence comprises a bacterial promoter, in specific embodiments. The sensor sequence may comprise part or all of one or more promoters from Escherichia coli Nissle 1917. In some cases, the calprotectin-responsive sequence comprises a functional part or all of the regulatory sequences of the ribosomal protein L31/L36 operon (ykgM, ykg ( ), the siderophore enterobactin (ent) operon, and/or ABC transporter operon (abt). The calprotectin-responsive sequence may be activated by zinc limitation and/or may comprise one or more zinc-uptake-regulator sites. Any indirect calprotectin-responsive sequences may be directly or indirectly sensitive to a metal to which calprotectin binds, such as free zinc, iron, manganese, or a combination thereof.

In various embodiments, the systems, methods, and compositions utilize one or more characteristics that enhances the range of expression of the calprotectin-responsive sequence. In specific cases, the level of expression when the promoter is not activated by zinc limitation is reduced. In certain cases, the level of expression when the promoter is not activated by zinc limitation is high because a repressor in the genome of the background is too low and imbalanced stoichiometrically. Therefore, in specific embodiments, when the calprotectin-responsive sequence is being expressed from a multicopy plasmid, the repressor is also expressed from the same or similar type of plasmid. In specific embodiments, the repressor is the Zur repressor and the zur gene is co-expressed on the same plasmid as the calprotectin-responsive sequence, such as the ykgMO promoter. The promoter that expresses the zur gene may be of a specific type.

In particular embodiments, the systems, methods, and compositions include a memory switch for the inflammation biosensor. In specific embodiments, the memory switch allows the biosensor to become sustainably activated upon sensing inflammation for future reporting in stool. In specific embodiments, the memory switch stays active even when zinc concentrations are restored to normal (for example, when inflammation is reduced). In particular embodiments, the calprotectin-responsive sequence driving expression of a detectable readout or therapeutic gene is present in the bacteria along with the memory switch. In specific embodiments, the memory switch comprises an expression construct separate from the calprotectin-sensor sequence driving expression of a detectable readout gene or in some cases a therapeutic gene, and both may or may not be on the same vector, for example. In specific embodiments, the memory switch comprises an expression construct wherein the detectable readout gene is in a reverse orientation such that the detectable readout gene cannot produce a detectable readout gene product, and the detectable readout gene is flanked by sequences that can be recognized by any one or more proteins having DNA inversion activity, including an integrase that recognizes attB and attP sequences, for example. Upon activation of the calprotectin-responsive sequence in the presence of calprotectin and low zinc levels, the one or more proteins having DNA inversion activity are expressed that then act on the sequences that can be recognized by them, thereby reversing the orientation of the detectable readout gene.

In some embodiments, methods of treating a gastrointestinal inflammatory condition, including treating intestinal inflammation, are encompassed herein. In specific embodiments, the calprotectin-responsive sequence regulates expression of a therapeutic gene that encodes a therapeutic gene product. In specific embodiments, the therapeutic gene is IL-10 or a fusion of IL-10 with a facilitating carrier protein to enhance secretion of the therapeutic gene product from the bacteria. In specific cases, the facilitating carrier protein is YebF. In specific embodiments, the methods of treatment utilize the memory switch analogous to use with the detectable readout gene. In any event, following providing to an individual in need thereof (including an individual known to have a gastrointestinal inflammatory condition or suspected of having a gastrointestinal inflammatory condition or at risk thereof), one or more symptoms of the gastrointestinal inflammatory condition is treated. In specific embodiments, the individual is also provided the system utilizing the detectable readout, and the feces of the individual is examined for the signal for inflammation. Such a monitoring step may occur prior to, at the same time as, and/or subsequent to use of the system with the therapeutic gene product. Although in some cases the gastrointestinal inflammatory condition is caused by a disease, in other cases the gastrointestinal inflammatory condition is caused by a pathogen. In specific cases, the pathogen may be one or more of C. difficile, C. concisus, F. nucleatum, B. fragilis, F. varium, Mycobacterium avium subspecies paratuberculosis, adherent-invasive Escherichia coli, Campylobacter species, Listeria monocytogenes, Candida albicans, Staphylococcus aureus, Streptococcus pyogenes, Pseudomonas aeruginosa, and Escherichia coli. In specific embodiments, the gastrointestinal inflammatory condition is caused by a pathogen and the individual may or may not be a patient in a hospital or a resident of a living facility including for long-term, such as a nursing home; in such cases, use of the system of the disclosure may or may not occur on a regular basis.

In particular embodiments, the readout product is a detectable colorimetric, ultraviolet, ultrasound, and/or fluorescent marker. The readout product may be one or more of the following: violacein, one or more chromoproteins, one or more carotenoids, one or more phycobilins, one or more anthocyanins, and/or indigo. In specific cases, the readout product is green fluorescence protein, yellow fluorescent protein, blue fluorescent protein, mCherry, or cyan fluorescent protein. The readout product may be detectable upon conversion by an enzyme of a substrate to a detectable product. In specific cases, the readout product is detectable upon conversion by an enzyme of a pro-dye to a visible dye, as one example.

In certain embodiments, the providing step occurs orally and may be performed by the individual. The providing step may or may not occur on a regular basis. The providing step may be performed once or it may occur on a regular basis, such as daily or weekly or monthly. The providing step may occur randomly, such as at the whim of the individual. It may occur during the presence or absence of one or more symptoms of intestinal inflammation. The providing step may occur after the presence of one or more symptoms of intestinal inflammation, such as to monitor efficacy of a therapeutic for the intestinal inflammation. The providing step may occur both before and after onset of one or more symptoms of intestinal inflammation. The examining step of the feces for the detectable readout product may or may not be performed by the individual. In some cases when the readout product is detected, the individual obtains treatment for the intestinal inflammation, which may or may not be from inflammatory bowel disease (IBD), including Crohn's Disease (CD) or ulcerative colitis (UC), as examples. In some cases when the readout product is detected, the individual receives treatment of the inflammation or cancer prior to onset of one or more symptoms.

In one embodiment there is a non-pathogenic bacteria or population thereof (which may be of any kind, such as any Eschercia sp., Lactobacillus sp., Lactococcus sp., Bacteroides sp., or Bacillus sp., including E. coli, Lactobacillus reuteri, Lactococcus lactis, Bacteroides thetaiotamicro, or Bacillus subtilis) comprising an engineered polynucleotide, said polynucleotide comprising one or more direct calprotectin-responsive sequences or indirect calprotectin-responsive sequences operably linked to a sequence that encodes a detectable readout product. In certain embodiments, the bacteria are any bacteria that contain the ykgMO) operon. The calprotectin-responsive sequence is a bacterial promoter, in some cases and may comprise a functional part or all of one or more promoters from Escherichia coli Nissle 1917. The calprotectin-sensor sequence may comprise a functional part or all of ribosomal protein L31/L36 operon (ykgM, ykg ( ), the siderophore enterobactin (ent) operon, and/or ABC transporter operon (abt) promoters. The indirect calprotectin-responsive sequences may be directly or indirectly sensitive to a metal to which calprotectin binds, such as free zinc, iron, manganese, or a combination thereof. In particular embodiments, the readout product is a detectable colorimetric, ultraviolet, ultrasound, or fluorescent marker. The readout product may be one or more of the following: violacein, one or more carotenoids, one or more phycobilins, one or more chromoproteins, one or more anthocyanins, and/or indigo. In some cases, the readout product is green fluorescence protein, yellow fluorescent protein, blue fluorescent protein, one or more phycobilins, one or more anthocyanins, or cyan fluorescent protein.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIGS. 1A-1K. Selection of the ykgMO promoter to sense calprotectin levels and sensitivity of ygkMO, ent and abt biosensors to metal starvation. 1A: Sensitivity of ykgMO (50S Ribosomal Protein L36/L31 type B) promoter (P_{ykg}) biosensor to calprotectin; 1B-ID: Sensitivity of ent (1B), abt (1C), and ykg (1D) promoter constructs to TPEN in M9 media detected by Flow Cytometer; 1E: Minimum inhibitory concentration (MIC) of calprotectin on E. coli Nissile1917 (referred to EcN) in LB media; IF: Upregulation of calprotectin responsive promoters with TPEN induction in LB media verified by RT-qPCR; 1G: Sensitivity of ent promoter construct to TPEN in M9 media detected by plate reader; 1H: Sensitivity of abt promoter construct to TPEN in M9 media detected by plate reader; 1I: Sensitivity of ykg promoter construct to TPEN in M9 media detected by plate reader; 1J: Upregulation of calprotectin responsive ykgMO) promoter with TPEN induction in M9 media verified by RT-qPCR; 1K: Quenching of different ions on P_{ykg} biosensor (EcN/pBSI0). Equimolar (1.5 μM) or 10×-excess (15 μM) of Zn²⁺, Mn²⁺, and Fe²⁺ were added to P_{ykg} biosensor co-cultured with 40 μg/mL calprotectin in M9 media. 1.5 μM (equivalent metal binding capacity of 40 μg/mL calprotectin), 3 μM, or 30 μM co-cultured with E. coli Nissle 1917 biosensors in pure culture. Calprotectin only-N=9. Metals-N=3, with two technical replicates per sample. ***-p<0.0001; N=3, with two technical replicates per sample. * p<0.05, ** p<0.01, *** p<0.001;

FIGS. 2A-2G. Optimization of the ykg promoter construct for use as a calprotectin biosensor. 2A: Schematic diagram of low basal expression biosensor plasmid with Zur box (pBSI1); 2B: Fluorescence of EcN/pBSI1 with Zur box under control of different strength constitutive promoters; 2C: Comparison of sfgfp basal expression by RT-qPCR; 2D: Schematic diagram of memory circuit biosensor BSIM; 2E Comparison of biosensor EcN/pBSI1 (BSI1) and EcN/pBSIM (BSIM) stability in Zn2* quenched condition; 2F Transcription of sfgfp of BSI1 and BSIM in Zn2-quenched condition; 2G: Primers N1+N2 were used for the non-flipping sfgfp gene detection. Primers N1+N3 were used for the flipped sfofp gene detection. Memory circuit biosensor EcN/pBSIM was induced with 30 μM TPEN in LB media for 0, 2, 4, 6, and 8 h, following the plasmid was isolated for PCR test. * p<0.05, ** p<0.01, *** p<0.001, *** p<0.0001.

FIGS. 3A-3M. Genetic memory circuit biosensor senses intestinal inflammation in vivo. 3A: Experimental design of DSS-induced gut inflammation in mouse (IBD animal model). 6-week-old C57BL/6 mice (5 female and 5 male mice in each group) were given water with or without 1-3% DSS for 6 days before oral gavage with memory circuit sensor bacteria. After 4-6 h, samples were collected from the mice, processed, and analyzed by using qPCR and plating to measure sensor activation fold and percentage; 3B: Schematic diagram of sensor off (before gavage) and sensor on (at the mice gut point with inflammation); 3C: Weight change of mice after 1-3% DSS treated; 3D: Colon length of mice after 1-3% DSS treated; 3E: Concentration of calprotectin in the mice colon contents detected by ELISA; 3F: Activation fold of memory circuit biosensor (fold change of flipped sfgfp gene) in the colon of DSS treated mice detected by qPCR; 3G: Correlations of biosensor activation (fold change of flipped sfgfp gene) and colon calprotectin concentration. 3H: Activation percentage of memory circuit biosensor (green colonies) in the colon of DSS treated mice detected by plating; 31: Experimental design of (′. difficile-induced gut inflammation in mouse (CDI animal model). Briefly, 6-week-old C57BL/6 mice (10 female and 10 male mice in each group) were treated with five mix antibiotics cocktail for four days, following intraperitoneal injection of one dose of clindamycin and 10⁴-10⁵ C. difficile R20291 spores infection. After two days post infection, mice were gavaged with 109 CFU of sensor bacteria. After 4-6 h, colon samples were collected and analyzed; 3J: Weight change of mice after C. difficile R20291 spores infection (CDI); 3K: Activation fold of memory circuit biosensor (fold change of flipped sfgfp gene) in the colon of CDI mice detected by qPCR; 3L: Concentration of calprotectin in the colon of CDI mice detected by ELISA; 3M: Correlations of biosensor activation (fold change of flipped sfgfp gene) and colon calprotectin concentration in CDI mice. * p<0.05, ** p<0.01, *** p<0.001, *** p<0.0001.

FIGS. 4A-4C. Genetic memory circuit biosensor senses intestinal inflammation in vivo (extended from FIG. 3). 4A: Activation fold of memory circuit biosensor (fold change of flipped sfgfp) from 5% DSS treated mice colon samples detected by qPCR. 6-week-old C57BL/6 mice (5 female and 5 male mice in each group) were given water with or without 5% DSS for 5 days before oral gavage with memory circuit sensor bacteria. After 4-6 h, samples were collected from the mice, processed, and analyzed by using qPCR; 4B: Concentration of calprotectin in the DSS treated mice colon contents detected by ELISA; 4C: Activation fold of memory circuit biosensor (fold change of flipped sfgfp) from C. difficile VPI10463 infected mice (10⁴ spores/mouse) colon samples detected by qPCR. Briefly, 6-week-old C57BL/6 mice were treated with five mix antibiotics cocktail for four days, following intraperitoneal injection of one dose of clindamycin and 10⁴ C. difficile VPI10463 spores infection. After two days post infection, mice were gavaged with 10⁹ CFU of sensor bacteria. After 4-6 h, colon samples were collected and analyzed. 3 female and 3 male mice in the control group and 5 female and 5 male mice in the experimental group were used (2 mice died before biosensor test). * p<0.05, ** p<0.01, *** p<0.001, *** p<0.0001.

FIGS. 5A-5G. Secretion of functionally active human IL10 in response to inflammation. 5A: Diagram of therapeutic sensor plasmid pBSIT1 with secreted IL10 fusion gene (yebl-hll.10); 5B: secIL 10 expression of therapeutic sensor EcN/pBSIT1 (BSIT1) induced with 0, 5, 10 and 20 μM TPEN for 2 and 4 h; 5C: Secretion of secIL 10 in EcN/pBSIT1 (BSIT1) compared to hIL 10 leaking expression in AmrcA/pBSIT0; 5D: Activity test of secIL 10 with HEK-Blue IL 10 reporter cell line; 5E: Diagram of secIL 10 stable expression sensor plasmid pBSIT2 (sensor BSIT2) and memory circuit therapeutic sensor plasmid pBSI™ (sensor BSI™) structure; 5F: Comparison of secIL10 expression plasmid stability in therapeutic biosensor BSIT1 (EcN/pBSIT1) and BSIT2 (EcNAasd/pBSIT1); 5G: Concentration of secIL 10 in the supernatants of BSIT2 and BSI™ detected by ELISA. * p<0.05, ** p <0.01, *** p<0.001, *** p<0.0001.

FIGS. 6A-6H. Therapeutic biosensor ameliorates intestinal inflammation in vivo. 6A: Experimental design of therapeutic biosensors applied in DSS-induced gut inflammation in mouse (IBD animal model) to ameliorate intestinal inflammation. 6-week-old C57BL/6 mice (10 male mice in each group) were orally gavaged 2×10⁹ therapeutic sensor bacteria or 100 μl PBS for three days, following mice were treated with or without 3% DSS for 11 days. 2×10⁹ therapeutic sensor bacteria or 100 μl PBS were gavaged every two days. On day 11, mice were euthanized and colon samples were collected; 6B: Weight change of mice in different treated groups; 6C: Percentage of mice with bloody feces in different treated groups; 6D: Concentration of calprotectin in mice feces from different treated groups; 6E: Colon length of mice in different treated groups; F: Histology of colon tissue with H&E staining; * p<0.05, ** p<0.01, *** p<0.001.

FIGS. 7A-7C. Memory circuit biosensor detection based on bfmo gene. 7A: Sketch map of memory circuit biosensor with bfmo gene. 7B: In vitro test of bfmo gene-based memory circuit biosensor. bfmo gene-based biosensor was cultured in M9 media with tryptophan induced or noninduced by TPEN. 7C: In vivo test of bfmo gene-based memory circuit biosensor in DSS animal model. Colon and cecum contents were collected, and then were diluted and plated on LB plates with tryptophan.

FIGS. 8A-8F. Genetic memory circuit biosensor expresses reporter sfGFP stably after TPEN removal. 8A: Memory circuit biosensor EcN/pBSIM was induced by 1.5 and 3 μM TPEN in M9 media. EcN/pBSIM was cultured to early log phase (OD-0.4-0.5), following final concentration of 1.5 and 3 μM TPEN were added to induce sfgfp expression; 8B: Activation fold of memory circuit biosensor induced by 1.5 and 3 μM TPEN in M9 media at 4 h induction; 8C: Stable expression of sfgfp in the memory circuit biosensor after TPEN remove at 4 h induction. After 4 h TPEN induction, cell cultures from FIG. S3A were centrifuged and resuspended in the same volume of fresh M9 media. Following, fluorescence and OD600 nm were detected for 10 h; 8D: Activation fold of memory circuit biosensor after TPEN remove. After 4 h extended incubation (TPEN remove), cell cultures from FIG. S3C were used for activation fold analysis of memory circuit biosensor by qPCR; 8E-8F: After 4 h TPEN induction, cell cultures from FIG. S3A were 1% back diluted into fresh M9 media without TPEN. Following, fluorescence and OD600 nm were detected for 10 h (8E), and the activation fold of memory circuit biosensor was analyzed at 8 h growth (8F). * p<0.05, ** p<0.01, *** p<0.001, *** p<0.0001.

FIGS. 9A-9B. Correlations of DSS concentration, calprotectin concentration, and biosensor activation.

FIGS. 10A-10C. Detection of activated memory circuit biosensor from mice intestine by plating. 10A-10B: Activation percentage of memory circuit biosensor (green colonies) induced with 30 μM TPEN in LB media in vitro measured by plating; 10C: Activation percentage of memory circuit biosensor (green colonies) from DSS treated mice colon contents. * p<0.05, ** p <0.01, *** p<0.001, *** p<0.0001.

FIGS. 11A-11D. Growth profiles of therapeutic sensors with TPEN induction. 11A: Growth profile of therapeutic sensor BSIT1 (EcN/pBSIT1) in LB media with TPEN induction; 11B: Growth profile of EcNAasd in LB media; 11C-11D: Growth profiles of therapeutic sensor BSIT2 (EcNAasd/pBSIT2) and memory circuit therapeutic sensor BSI™ (EcNAasd/pBSI™+pBSIM1) in LB media with TPEN induction.

FIGS. 12-12B. Stability of secIL10 expression plasmid in therapeutic biosensors. Therapeutic biosensor BSIT1 (EcN/pBSIT1) and BSIT2 (Aasd/pBSIT2) were cultured in LB media and LB+10 μM TPEN without antibiotic added, respectively. The biosensors were passaged 5 times, following 1 μl of 10-fold serial dilution cultures were spotted on LB and LB+Cm plates (12A). To evaluate the percentage of lost plasmid in biosensors, biosensor cultures from 5 passaged were plated on LB plates firstly, then a single colony was replica-cultured in LB and LB+Cm plates (12B).

FIG. 13. Schematic diagram of engineered biosensors based on colitis biomarker calprotectin for colitis diagnosis, recording and treatment. The engineered memory circuit biosensor sense and respond to colitis biomarker calprotectin and produce inheritable signals (plasmid DNA recording and colorimetric output) to monitor gut inflammation. Moreover, the engineered biosensor EcN can express and secrete the secIL10 (YebF-IL10) induced by calprotectin and thereby ameliorate colitis.

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of” or “consist of′ one or more sequences of the invention, for example. Some embodiments of the invention may consist of or consist essentially of one or more elements, method steps, and/or methods of the invention. It is contemplated that any method or composition described herein can be implemented with respect to any other method or composition described herein.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

It is contemplated that any embodiment discussed in this specification can be implemented with respect to any method or composition of the invention, and vice versa. Furthermore, compositions of the invention can be used to achieve methods of the invention.

Throughout this application, the term “about” is used according to its plain and ordinary meaning in the area of cell and molecular biology to indicate that a value includes the standard deviation of error for the device or method being employed to determine the value.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In specific embodiments, a vector is engineered through recombinant nucleic acid technologies, and a cell is engineered through transfection or transduction of an engineered vector.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “subject,” as used herein, generally refers to an individual having a that has or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasias, or cancer. The subject may being undergoing or having undergone treatment. The subject may be asymptomatic. The subject may be healthy individuals but that are desirous of prevention of cancer. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

As used herein “treatment” or “treating,” includes any beneficial or desirable effect on the symptoms or pathology of a disease or pathological condition, and may include even minimal reductions in one or more measurable markers of the disease or condition being treated, e.g., a gastrointestinal inflammatory condition. Treatment can involve optionally either the reduction or amelioration of one or more symptoms of the disease or condition, or the delaying of the progression of the disease or condition. “Treatment” does not necessarily indicate complete eradication or cure of the disease or condition, or associated symptoms thereof. Treatment may encompass reduction in inflammation, and in specific cases the inflammation is measured by standard means in the art.

The term “calprotectin-responsive sequence(s)” as used herein refers to polynucleotide sequence in which the protein calprotectin (including in a natural environment) directly or indirectly manipulates the sequence to result in modulation of expression of a gene product. In specific embodiments, calprotectin manipulation results in upregulation of expression of a gene product. In specific embodiments, the calprotectin-responsive sequence represents a zinc level in the environment in which the bacteria comprising a polynucleotide comprising the calprotectin-responsive sequence resides.

### I. GENERAL EMBODIMENTS

Embodiments of the disclosure include microbial biosensors for inflammation detection in an individual, including at least gastrointestinal inflammation of any kind. Detection of the microbial biosensor provides clinical information for the individual, including onset of inflammation itself or any symptom(s) related to inflammation in general or an IBD specifically. The microbial biosensor in at least some cases provides clinical information that is rapid, non-invasive, and is utilized in real-time, including in an at-home setting, as an example. Such a use reduces the need for clinical contact and facilitates patient compliance. In at least some cases, routine use of the system, including repeated administrations, facilitates reduction of day-to-day variance.

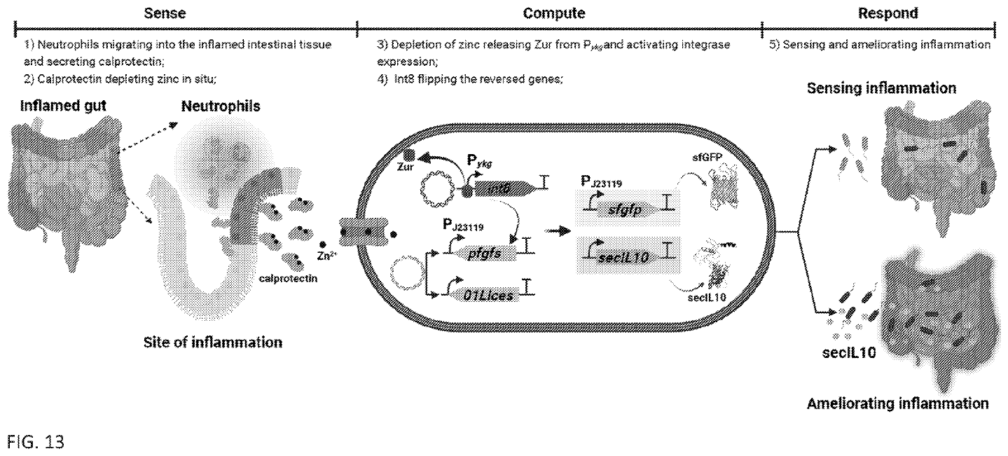

Embodiments of the disclosure include engineered biosensors, such as calprotectin-responsive microbial biosensors, that are capable of detecting and recording gut inflammation and in situ secreting therapeutic recombinant human IL10 (secIL10) carried by YebF to ameliorate intestinal inflammation (FIG. 13). In certain embodiments, to stabilize sensor output within the gastrointestinal tract where disease states can be variable, a genetic memory circuit is integrated into the biosensor to allow for continuous output upon activation of the sensor despite variations in calprotectin and zinc content throughout the gastrointestinal tract. In certain embodiments, a self-tunable engineered calprotectin-based biosensor detects and records intestinal inflammation. In certain embodiments, the self-tunable engineered calprotectin-based biosensor attenuates intestinal inflammation, including in IBD subjects. This allows for a new therapeutic platform for IBD, CDI, and other inflammatory diseases.

In particular embodiments, the biosensor is capable of sensing inflammation at the site of inflammation, such as at the intestinal lining, rather than before or after the site of inflammation. In particular embodiments, the biosensor generates an output, which may include a readout and/or therapeutic agent. The output may be sustained after the biosensor senses the inflammation, including by the genetic memory switch disclosed herein. In certain embodiments, the biosensor delivers a therapeutic gene product at the site of inflammation.

In specific embodiments, the biosensors are sensitive to one or more inflammation biomarkers, including one or more gut inflammation biomarkers. The disclosure provides for a microbial (including bacterial) biosensor that senses an inflammatory level of at least one disease biomarker and produces a detectable output based on the presence of the disease biomarker(s). In at least some cases, the inflammatory level of one or more disease biomarkers is recognized based on inflammation-induced promoters in a diagnostic gene expression system. In specific embodiments, a detectable output based on inflammation biomarker-induced gene expression of a detectable gene product is interpreted by the individual with an inflammatory medical condition or an infection. In specific cases, the detectable output comprises a detectable characteristic of the individual's feces, such as a change in color or the presence of fluorescence, as examples. As an example, a change in color may be a detectable dye pigment in the feces, or fluorescence in the feces may be detected based on suitable light conditions.

In certain embodiments, the microbial biosensor system detects a particular compound associated with the gut inflammation disease. As an example, the microbial biosensor system may detect a compound secreted by neutrophils. As a further example, the microbial biosensor system may recognize a secreted antimicrobial peptide from neutrophils, such as calprotectin that is a heterodimer with each peptide having specificity to certain metals. Calprotectin makes up approximately 50% of total neutrophil granule proteins, is bacteriostatic, and sequesters zinc, manganese, and iron. Thus, in specific embodiments calprotectin sensitivity is associated with a biomarker in fecal testing. In some cases, calprotectin is employed in the context of the methods of the disclosure as being a marker for any kind of inflammation. In alternatives, an inflammatory biomarker other than calprotectin is employed.

In particular embodiments, medical conditions associated with high calprotectin levels are detected utilizing methods of the disclosure. In specific cases, Clostridium difficile infection results in high calprotectin levels and may be the subject of methods for detecting onset of one or more symptoms and/or for methods of treating encompassed herein.

In particular embodiments, the disclosure concerns the development of an in-home inflammation monitoring system that would introduce a vast improvement for IBD flare detection, giving patients advanced warning that currently is not represented in IBD diagnostics. Embodiments include a synthetic probiotic that detects and reports calprotectin levels that are considered clinically relevant but prior to one or more major symptoms of disease. In one embodiment, the detection occurs through the linking of microbial promoters (including aerobic or anaerobic bacterial promoters) to the secretion of a detectable pigment. In specific embodiments, calprotectin-sensitive promoters are utilized. In specific embodiments of calprotectin-sensitive promoters, promoters of genes are utilized that are upregulated greater than at least two-fold by calprotectin induction (but in some cases below levels of calprotectin that are inhibitory to bacterial growth).

The microbial biosensor may be taken orally by a patient at home on a regular schedule, allowing the patient to monitor disease state in real-time on a much shorter timescale, with increased frequency of diagnostic administrations. In some embodiments, the biosensor is taken during remission of an inflammatory condition. In some embodiments, the biosensor is taken when the individual has active inflammation. In certain embodiments, the biosensor is taken when the individual is experiencing a flare-up of an inflammatory condition In certain embodiments, the biosensor is taken when the patient is undergoing symptoms of an inflammatory condition. In certain embodiments, the biosensor is taken when the patient is not undergoing symptoms of an inflammatory condition. This inflammation biosensor may be engineered to have partial or total repression of signal in the absence of stimulating calprotectin. In the presence of inflammatory levels of calprotectin anywhere in the gastrointestinal tract, production of the detectable readout is initiated, and then sustained and amplified over the course of passage throughout the GI tract. Positive signal is detected in excreted stool, such as within one or more days of taking the probiotic biosensor, giving the patient an earlier warning of potential inflammatory onset.

Examination of the feces for the detectable readout product may occur by any suitable method and in at least some embodiments is performed by the individual having the gastrointestinal inflammatory condition. Although in particular embodiments the individual makes the determination of the presence of inflammation based on readout in the stool, in some cases a medical practitioner may make the determination of the presence of the detectable readout product or may confirm the determination by the individual. In such cases, the medical practitioner may or may not do so in a virtual setting over the internet. In specific embodiments, the examination is visual and requires no manipulation of the feces. In alternative cases, the examination is visual and includes manipulation of the feces. For example, one may be required to manipulate the feces in order to detect the detectable readout, such as when a region of the feces in which the detectable readout product is present is internal within the feces and obscured to the naked eye. In specific embodiments, the toilet or receptacle in which the examination step is made comprises one or more compounds in the water that allows, facilitates, or enhances visualization of the detectable readout.

In embodiments of the disclosure, there is a method of determining a need for therapy for intestinal inflammation or cancer in an individual, comprising the steps of providing to the individual a population of non-pathogenic bacteria comprising an engineered polynucleotide comprising one or more direct calprotectin-responsive sequences or indirect calprotectin-responsive sequences operably linked to expression of a detectable readout product; and examining the feces of the individual for the detectable readout product.

In some embodiments, there is a method of monitoring a gastrointestinal inflammatory condition in an individual, including monitoring for the onset of one or more symptoms of the gastrointestinal inflammatory condition.

In some cases, there are methods of monitoring a therapy for a gastrointestinal inflammatory condition for an individual. The individual is provided a population of non-pathogenic bacteria comprising an engineered polynucleotide that comprises one or more direct calprotectin-sensor sequences (or indirect calprotectin-sensor sequences operably linked to expression of a detectable readout product. Following onset of one or more symptoms of the gastrointestinal inflammatory condition, the individual detects the detectable readout product upon examination of their feces. The individual is provided one or more therapies for the gastrointestinal inflammatory condition and continues over time to examine their feces for the detectable readout product. When the therapy is providing treatment of one or more symptoms of the gastrointestinal inflammatory condition, the detectable readout diminishes, including in some cases to a non-detectable level.

In particular embodiments, the method of monitoring intestinal inflammation in an individual (and/or treating intestinal inflammation in the individual) comprises the steps of (a) providing to the individual an effective amount of a population of non-pathogenic bacteria comprising at least two engineered polynucleotides, wherein: (1) a first said polynucleotide comprises one or more calprotectin-responsive sequences operably linked to expression of one or more proteins having DNA inversion activity; and (2) a second said polynucleotide comprises a reverse orientation of a gene product of interest flanked by attachment or recognition sites for the one or more proteins having DNA inversion activity, wherein exposure of the one or more proteins having DNA inversion activity to the second polynucleotide results in inversion of the reverse orientation of the gene product into an orientation of the gene product by which a functional gene product is produced; wherein: (b1) the gene product of interest is a detectable readout product, and the method further comprises examining the feces of said individual for the detectable readout product; and/or (b2) the gene product of interest is a therapeutic gene, and the intestinal inflammation is treated. In specific embodiments, the regulation of expression of the therapeutic gene is driven by a calprotectin-responsive sequence. In certain embodiments, the method comprises examining the feces of an individual for a detectable readout product, when the individual has been provided an effective amount of a population of non-pathogenic bacteria comprising at least two engineered polynucleotides, wherein: (1) a first said polynucleotide comprises one or more calprotectin-responsive sequences operably linked to expression of one or more proteins having DNA inversion activity; and (2) a second said polynucleotide comprises a reverse orientation of the detectable readout flanked by attachment or recognition sites for the one or more proteins having DNA inversion activity, wherein exposure of the one or more proteins having DNA inversion activity to the second polynucleotide results in inversion of the reverse orientation of the detectable readout into an orientation of the gene product by which a functional detectable readout is produced. In specific embodiments, the one or more proteins having DNA inversion activity comprise one or more proteins the sum of which are capable of inversion of DNA. In specific embodiments, the one or more proteins having DNA inversion activity comprises an integrase, Rci recombinase, or FimB and FimE, and regulatory proteins H-NS, Integration Host Factor (IHF) and Leucine responsive protein (LRP), as examples only. In alternative embodiments, instead of there being a first and second polynucleotides, the respective expression constructs are on the same polynucleotide.

In particular embodiments, the microbial biosensor system detects calprotectin at particular levels in the gut. The level in specific cases may be >50 μg/mL, >75 μg/mL, >100 μg/mL, >110 μg/mL, >120 μg/mL, >125 μg/mL, >130 μg/mL, >140 μg/mL, >150 g/mL,>175 μg/mL, and so forth, for example. In specific embodiments, the sensor works at the site of inflammation in the gastrointestinal system yet the readout occurs in the feces, such that the sensor is not actually sensing the feces environment but sensing inflammation prior to that in the system.

In particular embodiments, the microbial biosensor is ingested as non-pathogenic bacteria that comprise one or more engineered polynucleotides that have one or more direct calprotectin-sensor sequences or indirect calprotectin-responsive sequences operably linked to expression of a detectable readout product and/or a therapeutic gene product. In particular embodiments the bacteria are formulated as a probiotic of any kind. Such non-pathogenic bacteria may be ingested by the individual (including on a routine basis) so that the individual is ensured of detecting the presence of the detectable readout in their feces as it occurs and in at least some cases also to expose the individual to the practice and habit of monitoring their feces and becoming familiar with its day-to-day appearance. In specific embodiments, the bacteria are ingested every day, once or twice a day, every other day, once a week, one to three times a week, once a month, several times a month, and so forth. The bacteria may be ingested 1-2, 1-3, 1-4, 1-5, 1-6, 1-7, 1-8, 1-9, 1-10, 1-11, 1-12, 1-13, or 1-14 times a week, or any range derivable therein, in some cases.

The detectable readout of the system may be detectable in a variety of ways, as an example so long as an individual is not required to employ a medical practitioner for making the determination. In some cases, the detectable readout in the feces comprises a color change in the feces. The color may be of any color so long as it is distinguishable from the feces color. In other cases, the detectable readout in the feces comprises the presence of fluorescence, and such a determination may require particular light conditions to be able to identify the fluorescence (for example, turning off overhead or other lights in the room or using a fluorescence detection device).

In specific embodiments, the detectable readout is a specific pigment in the feces, such as a non-toxic pigment. In specific cases, one can utilize violacein, a non-toxic bacterially-derived pigment, although alternatives to violacein include anthocyanins, indigo and/or one or more carotenoids (for example, α-carotene, β-carotene, and/or lycopene), one or more chromoproteins, and/or one or more phycobilins (for example, phycocyanobilin). As one example, violacein production will shift fecal color, turning stool a purple hue that would be visible to the patient upon excretion. In specific embodiments, normal light or ultraviolet light may be utilized for detection of the detectable readout product. In specific cases, the light signal is transformed into an electrical signal as the readout mechanism. In certain cases, the bacteria may produce gas bubbles, such as that can be detected by ultrasound. In specific embodiments, an enzyme is utilized that makes a colorimetric change by interaction with a pro-dye being converted to a visible dye or another detectable change.

As an illustrative embodiment only, this disclosure encompasses a microbial biosensor that utilizes endogenous promoters of Escherichia coli Nissle 1917 to detect the presence of calprotectin, a neutrophil-source antimicrobial peptide. The primary function of calprotectin is to sequester zinc, iron, and/or manganese from the extracellular space. The endogenous promoters that are being used in the sensors have all demonstrated sensitivity to zinc deficiency and the metal-binding properties of calprotectin. The biosensors may have their sensing of calprotectin-induced zinc deficiency coupled with the expression of a colorimetric dye, such as violacein. The dye production may be enhanced to alter fecal pigment, giving patients a private and in-home method of monitoring and detecting intestinal inflammation.

In particular embodiments, upon detection of the detectable readout in the individual's feces, the individual may take action to treat one or more symptoms of the gut inflammation. The action(s) may reduce the intensity of the symptom or delay the onset of one or more symptoms. Examples of treatment include one or more anti-inflammatoires, one or more antibiotics, one or more Aminosalicylates (5-ASAs), one or more corticosteroids, one or more immune modifiers (immunomodulators), and/or one or more biologic therapies. Specific compounds include metronidazole, ciprofloxacin, sulfasalazine, mesalamine, olsalazine, balsalazide, prednisone, azathioprine, cyclosporine, 6-mercaptopurine, tacrolimus, methotrexate, infliximab, infliximab-dyyb, or a combination thereof.

In particular embodiments, the treatment itself comprises the system of the disclosure configured to deliver a therapeutic gene products. In particular embodiments, one or more calprotectin-responsive sequences are operably linked to expression of one or more therapeutic genes, and an expression construct comprising same is present on a recombinant polynucleotide in bacteria of the disclosure. A therapeutically effective amount, such as 10⁶ to 10¹¹, of the bacteria are provided to an individual in need thereof. The individual may have a personal or family history of gastrointestinal inflammation or be at risk for gastrointestinal inflammation. The individual may or may not have utilized the biosensor bacteria of the disclosure. In particular embodiments, the individual is provided an effective amount of non-pathogenic bacteria comprising one or more engineered polynucleotides, wherein the polynucleotide(s) comprise: (a) one or more calprotectin-responsive sequences operably linked to expression of a therapeutic gene; and optionally (b) sequence of the zinc uptake regulator (Zur) repressor. The regulation of expression of the zur gene may or may not be by a constitutive promoter of any kind, including J23114 or J23109 merely as examples. The therapeutic gene and the zur gene may be on the same polynucleotide or on different polynucleotides. Although the therapeutic gene may be of any kind that can ameliorate at least one symptom of gastrointestinal inflammation, including at least one symptom of IBD or infection, for example, in specific cases the therapeutic gene is one or more of IL-10, Elafin, IL-22, IL-36, and anti-TNF nanobodies. In specific embodiments, the therapeutic gene is linked to a facilitating carrier protein that allows the therapeutic gene product to be secreted from the bacteria. In specific cases the facilitating carrier protein is YebF.

In particular embodiments when the bacteria comprise (a) one or more calprotectin-responsive sequences operably linked to expression of a therapeutic gene; and optionally (b) sequence of the zinc uptake regulator (Zur) repressor, the bacteria may also comprise a first polynucleotide that comprises one or more calprotectin-responsive sequences operably linked to expression of one or more proteins having DNA inversion activity; and (2) a second polynucleotide that comprises a reverse orientation of a gene product of interest flanked by attachment or recognition sites for the one or more proteins having DNA inversion activity, wherein exposure of the one or more proteins having DNA inversion activity to the second polynucleotide results in inversion of the reverse orientation of the gene product into an orientation of the gene product by which a functional gene product is produced. In certain embodiments, the first polynucleotide and/or second polynucleotide are incorporated into the genome of the bacteria. The polynucleotide(s) may be incorporated at a location in the genome to allow for expression of the polynucleotides. In certain embodiments, the genome of the bacteria is engineered to remove at least one essential gene from the genome. The essential gene(s) may be replaced in the bacteria via the first polynucleotide and/or second polynucleotide described herein. In some aspects, the first polynucleotide and/or second polynucleotide encodes an essential gene, which may be expressed when present in the bacteria. In some embodiments, the essential gene is incorporated into a plasmid described herein, including a plasmid that contains the calprotectin-responsive sequences, the DNA inversion protein-encoding sequences, or the functional gene product-encoding sequences. An essential gene may comprise a gene that, when removed from the genome of the bacteria, kills the bacteria or causes the bacteria to stop reproducing.

Embodiments of the disclosure encompass non-pathogenic bacterial composition, comprising one or more engineered polynucleotides, wherein: (1) a first said polynucleotide comprises one or more calprotectin-responsive sequences operably linked to expression of an integrase; and (2) a second said polynucleotide comprises a reverse orientation of a gene product of interest flanked by attachment sites for the integrase; wherein: (a) the gene product of interest is a detectable readout product; and/or (b) the gene product of interest is a therapeutic gene, including a therapeutic gene that encodes a therapeutic gene product that may be operably linked to a facilitating carrier protein to facilitate secretion of the therapeutic gene product from the bacteria. In the bacteria, one or more calprotectin-responsive sequences may be operably linked to expression of the gene product of interest. In specific embodiments, either polynucleotide further comprises expression of the zinc uptake regulator (Zur) repressor that may or may not be regulated in expression by a constitutive promoter. The gene product of interest may be a detectable colorimetric, ultraviolet, ultrasound, and/or fluorescent marker, including at least one or more of the following: violacein, one or more carotenoids, one or more phycobilins, one or more anthocyanins, and indigo. In specific cases, the readout product is green fluorescence protein, yellow fluorescent protein, blue fluorescent protein, one or more phycobilins, one or more anthocyanins, or cyan fluorescent protein.

### II. POLYNUCLEOTIDE SEQUENCES FOR EMBODIMENTS HEREIN

The disclosure includes embodiments wherein calprotectin-responsive regulatory sequence, such as a promoter, are utilized as a means for detection of calprotectin at a level that signals the onset of one or more symptoms of gut inflammation. In particular cases, sequestration of metals in the extracellular space and/or binding of calprotectin to a bacterial cell elicits activation of a bacterial promoter on a recombinant polynucleotide therein, resulting in the expression of a gene product that is detectable, such as detectable in feces. Alternatively, calprotectin alters the environment in such a way that elicits activation of a bacterial promoter resulting in the expression of a gene product that is detectable, such as detectable in feces. One such way that calprotectin alters the environment is by chelating metals, which may alter expression of calprotectin sensitive promoters via depletion of metals such as Zinc, Iron, or Manganese.

Thus, upon identification of a calprotectin-responsive promoter, the promoter may be operably linked to a polynucleotide encoding a readout gene product that is detectable. The detectable gene product may produce a product that is colorimetric, fluorescent, or light-sensitive. An expression construct comprising a calprotectin-sensitive promoter operably linked to expression of a polynucleotide encoding a detectable readout gene product may be utilized.

In some cases, a calprotectin-sensitive promoter is derived from a bacterial genome. Such a promoter may be utilized in its entirety, or the promoter may be modified compared to the endogenous bacterial promoter sequence. For example, a bacterial promoter may be truncated to modify the strength of the promoter or to reduce background expression levels of the promoter. Such modifications may increase the signal to noise ratio of the promoters, allowing for increased sensitivity.

In specific embodiments, the promoters are from E. coli Nissle. As particular embodiments, promoters in E. coli Nissle 1917 that are sensitive to IBD-associated biomarker(s) are utilized. Specific examples are as follows: ribosomal protein L31/L36 operon (ykgMO), the siderophore enterobactin (ent) operon, and/or an ABC transporter operon (abt).

Examples of specific promoters useful for embodiments described herein, which in certain embodiments, are calprotectin-sensitive promoters, are as follows:

E. coli Nissle 1917 L36 Accessory Protein Promoter-ykgMO

(SEQ ID NO: 1)
taacggcaataaactgttcacttcagTGATATTTAAAATATGCATCCTC
TCCCTTTTTTGTAAGTAATTATTATATCCGTGGGAGAGGAATACACATT
GTCAGGTAATCAATCATGCTGCAATAAATCATCGGCCAGTAAAGTGGAG
ATAGCCTCCATTCTCGAAAAATCCATACTCTCAGCGAAACCATCATCAA
TCACTCATCCAGGCGTTTATGGGAGCGTCGCCAATGGCTGCTAACAATG
CCAGACTTCCCCGTTGCGGAAATTCCACATCCCACAAATAGTCACAGTG
ATTGGGTGTTGAAATGATCCGGATGAGCATGTATCTTTAcggttatgtt
ataacataacaggtaaaaatg

E. coli Nissle 1917 ABC Transporter Promoter-P_{abt}

(SEQ ID NO: 2)
aacagacccgaagaaaatgaaatataagAAAAGATCAACGAGTGAAGAA
AAAGTTCAAAAAATGGCTGCCGGGGAGGAAGGAAAGTACCGGATGGAAA
GAGTCCCCCCTAAAGCAGACTGACAGACATAACAAATCCCCGGGGGGAT
TTGTGTATAAGAGACAGTACTTATCTGGAGGTAATTGCAATATCTCTGT
GAACTTACACAGGGTGGGCTTACCGCATACACTGACACTTAGCGGATCG
ACAGAACATTATTAACAGAGCATCACTGAACGCTACATAATCAGAGTTG
CATAAATAAAATGTTATTAACATcacaatcacaacatttcga

E. coli Nissle Enterobactin Synthase Promoter-entCEBA-ybdB

(SEQ ID NO: 3)
aagtcagatcctgttattaatgaagTTAATGCTTCTCATTTTCATTGTA
ACCACAACCAGATGCAACCCCGAGTTGCAGATTGCGTTACCTCAAGAGT
TGACATAGTGCGCGTTTGCTTTTAGGTTAGCGACCGAAAATATAAATGA
TAATCATTATTAAAGCCTTTATcattttgtggaggatgat

J23114 promoter

(SEQ ID NO: 4)
tttatggctagctcagtcctaggtacaatgctagc

J23109 promoter

(SEQ ID NO: 5)
tttacagctagctcagtcctagggactgtgctagc

J23119 promoter

(SEQ ID NO: 6)
ttgacagctagctcagtcctaggtataatgctagc

Bacillus subtilis ytiA promoter

(SEQ ID NO: 7)
atgccatgaggtttacgatgtgaaacagagggaaggatataagaa
gattgacaaaacgtttgtaaggttataaaataaaacgtaattatt
acgatttattgaaaggagatacccatgaaa

Bacillus subtilis yhzA promoter

(SEQ ID NO: 8)
acaaagcggtatgcttgcttttttgaatggtgaatgatacatttt
aaatcgtaacaatttcgatttaaagggaggctatgtgacttggct
aaatcgtaatattacgattt

All or a functional portion of a promoter controlling the expression of ribosomal proteins L36, L33, L31, and S14, including promoters found in E. coli. S. typhi, K. pneumoniae, V. cholerae, Y. pestis, B. subtilis, S. aureus, L. monocytogenes, L. innocua, E. faecalis, S. pneumoniae, S. mutans, S. pyrogenes, and/or L. lactis.

In some embodiments, the promoter comprises all or a functional portion of any of SEQ ID NOS: 1-8. The functional portion may be a sufficient portion of the promoter sequence capable of expressing the gene of interest in the conditions relevant to the embodiment, including when sensing inflammation. In some embodiments, the promoter comprises 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or any range derivable therein, of any of SEQ ID NOS: 1-8.

Examples of polynucleotides useful for embodiments described herein, which can comprise the promoter, gene of interest, memory circuit, reporter gene, and/or therapeutic gene, are as follows:

pBSI0

(SEQ ID NO: 9)
GCTGGCGTTTTTCCATAGGCTCCGCCCCCCTGACGAGCATCACAA
AAATCGACGCTCAAGTCAGAGGTGGCGAAACCCGACAGGACTATA
AAGATACCAGGCGTTTCCCCCTGGAAGCTCCCTCGTGCGCTCTCC
TGTTCCGACCCTGCCGCTTACCGGATACCTGTCCGCCTTTCTCCC
TTCGGGAAGCGTGGCGCTTTCTCAATGCTCACGCTGTAGGTATCT
CAGTTCGGTGTAGGTCGTTCGCTCCAAGCTGGGCTGTGTGCACGA
ACCCCCCGTTCAGCCCGACCGCTGCGCCTTATCCGGTAACTATCG
TCTTGAGTCCAACCCGGTAAGACACGACTTATCGCCACTGGCAGC
AGCCACTGGTAACAGGATTAGCAGAGCGAGGTATGTAGGCGGTGC
TACAGAGTTCTTGAAGTGGTGGCCTAACTACGGCTACACTAGAAG
GACAGTATTTGGTATCTGCGCTCTGCTGAAGCCAGTTACCTTCGG
AAAAAGAGTTGGTAGCTCTTGATCCGGCAAACAAACCACCGCTGG
TAGCGGTGGTTTTTTTGTTTGCAAGCAGCAGATTACGCGCAGAAA
AAAAGGATCTCAAGAAGATCCTTTGATCTTTTCTACGGGGTCTGA
CGCTCAGTGGAACGAAAACTCACGTTAAGGGATTTTGGTCATGAC
TAGTGCTTGGATTCTCACCAATAAAAAACGCCCGGCGGCAACCGA
GCGTTCTGAACAAATCCAGATGGAGTTCTGAGGTCATTACTGGAT
CTATCAACAGGAGTCCAAGCGAGCTCGATATCAAATTACGCCCCG
CCCTGCCACTCATCGCAGTACTGTTGTAATTCATTAAGCATTCTG
CCGACATGGAAGCCATCACAGACGGCATGATGAACCTGAATCGCC
AGCGGCATCAGCACCTTGTCGCCTTGCGTATAATATTTGCCCATG
GTGAAAACGGGGGCGAAGAAGTTGTCCATATTGGCCACGTTTAAA
TCAAAACTGGTGAAACTCACCCAGGGATTGGCTGAGACGAAAAAC
ATATTCTCAATAAACCCTTTAGGGAAATAGGCCAGGTTTTCACCG
TAACACGCCACATCTTGCGAATATATGTGTAGAAACTGCCGGAAA
TCGTCGTGGTATTCACTCCAGAGCGATGAAAACGTTTCAGTTTGC
TCATGGAAAACGGTGTAACAAGGGTGAACACTATCCCATATCACC
AGCTCACCGTCTTTCATTGCCATACGAAATTCCGGATGAGCATTC
ATCAGGCGGGCAAGAATGTGAATAAAGGCCGGATAAAACTTGTGC
TTATTTTTCTTTACGGTCTTTAAAAAGGCCGTAATATCCAGCTGA
ACGGTCTGGTTATAGGTACATTGAGCAACTGACTGAAATGCCTCA
AAATGTTCTTTACGATGCCATTGGGATATATCAACGGTGGTATAT
CCAGTGATTTTTTTCTCCATTTTAGCTTCCTTAGCTCCTGAAAAT
CTCGATAACTCAAAAAATACGCCCGGTAGTGATCTTATTTCATTA
TGGTGAAAGTTGGAACCTCTTACGTGCCGATCAACGTCTCATTTT
CGCCAGATATCGACGTCTAAGAAACCATTATTATCATGACATTAA
CCTATAAAAATAGGCGTATCACGAGGCCCTTTCGTCTTCACCTCG
AGTAACGGCAATAAACTGTTCACTTCAGTGATATTTAAAATATGC
ATCCTCTCCCTTTTTTGTAAGTAATTATTATATCCGTGGGAGAGG
AATACACATTGTCAGGTAATCAATCATGCTGCAATAAATCATCGG
CCAGTAAAGTGGAGATAGCCTCCATTCTCGAAAAATCCATACTCT
CAGCGAAACCATCATCAATCACTCATCCAGGCGTTTATGGGAGCG
TCGCCAATGGCTGCTAACAATGCCAGACTTCCCCGTTGCGGAAAT
TCCACATCCCACAAATAGTCACAGTGATTGGGTGTTGAAATGATC
CGGATGAGCATGTATCTTTACGGTTATGTTATAACATAACAGGTA
AAAATGAAAGAGGAGAAATACTAGATGCGTAAAGGCGAAGAGCTG
TTCACTGGTGTCGTCCCTATTCTGGTGGAACTGGATGGTGATGTC
AACGGTCATAAGTTTTCCGTGCGTGGCGAGGGTGAAGGTGACGCA
ACTAATGGTAAACTGACGCTGAAGTTCATCTGTACTACTGGTAAA
CTGCCGGTACCTTGGCCGACTCTGGTAACGACGCTGACTTATGGT
GTTCAGTGCTTTGCTCGTTATCCGGACCATATGAAGCAGCATGAC
TTCTTCAAGTCCGCCATGCCGGAAGGCTATGTGCAGGAACGCACG
ATTTCCTTTAAGGATGACGGCACGTACAAAACGCGTGCGGAAGTG
AAATTTGAAGGCGATACCCTGGTAAACCGCATTGAGCTGAAAGGC
ATTGACTTTAAAGAAGACGGCAATATCCTGGGCCATAAGCTGGAA
TACAATTTTAACAGCCACAATGTTTACATCACCGCCGATAAACAA
AAAAATGGCATTAAAGCGAATTTTAAAATTCGCCACAACGTGGAG
GATGGCAGCGTGCAGCTGGCTGATCACTACCAGCAAAACACTCCA
ATCGGTGATGGTCCTGTTCTGCTGCCAGACAATCACTATCTGAGC
ACGCAAAGCGTTCTGTCTAAAGATCCGAACGAGAAACGCGATCAT
ATGGTTCTGCTGGAGTTCGTAACCGCAGCGGGCATCACGCATGGT
ATGGATGAACTGTACAAATGATGATAATAATCTAGACCAGGCATC
AAATAAAACGAAAGGCTCAGTCGAAAGACTGGGCCTTTCGTTTTA
TCTGTTGTTTGTCGGTGAACGCTCTCTACTAGAGTCACACTGGCT
CACCTTCGGGTGGGCCTTTCTGCGTTTATAGGTACCCGTGGATCC
TCTAGAGTCGACCGAGCGGTATCAGCTCACTCAAAGGCGGTAATA
CGGTTATCCACAGAATCAGGGGATAACGCAGGAAAGAACATGTGA
GCAAAAGGCCAGCAAAAGGCCAGGAACCGTAAAAAGGCCGCGTT

pBSI0-Pₑₙₜ

(SEQ ID NO: 10)
GCTGGCGTTTTTCCATAGGCTCCGCCCCCCTGACGAGCATCACAA
AAATCGACGCTCAAGTCAGAGGTGGCGAAACCCGACAGGACTATA
AAGATACCAGGCGTTTCCCCCTGGAAGCTCCCTCGTGCGCTCTCC
TGTTCCGACCCTGCCGCTTACCGGATACCTGTCCGCCTTTCTCCC
TTCGGGAAGCGTGGCGCTTTCTCAATGCTCACGCTGTAGGTATCT
CAGTTCGGTGTAGGTCGTTCGCTCCAAGCTGGGCTGTGTGCACGA
ACCCCCCGTTCAGCCCGACCGCTGCGCCTTATCCGGTAACTATCG
TCTTGAGTCCAACCCGGTAAGACACGACTTATCGCCACTGGCAGC
AGCCACTGGTAACAGGATTAGCAGAGCGAGGTATGTAGGCGGTGC
TACAGAGTTCTTGAAGTGGTGGCCTAACTACGGCTACACTAGAAG
GACAGTATTTGGTATCTGCGCTCTGCTGAAGCCAGTTACCTTCGG
AAAAAGAGTTGGTAGCTCTTGATCCGGCAAACAAACCACCGCTGG
TAGCGGTGGTTTTTTTGTTTGCAAGCAGCAGATTACGCGCAGAAA
AAAAGGATCTCAAGAAGATCCTTTGATCTTTTCTACGGGGTCTGA
CGCTCAGTGGAACGAAAACTCACGTTAAGGGATTTTGGTCATGAC
TAGTGCTTGGATTCTCACCAATAAAAAACGCCCGGCGGCAACCGA
GCGTTCTGAACAAATCCAGATGGAGTTCTGAGGTCATTACTGGAT
CTATCAACAGGAGTCCAAGCGAGCTCGATATCAAATTACGCCCCG
CCCTGCCACTCATCGCAGTACTGTTGTAATTCATTAAGCATTCTG
CCGACATGGAAGCCATCACAGACGGCATGATGAACCTGAATCGCC
AGCGGCATCAGCACCTTGTCGCCTTGCGTATAATATTTGCCCATG
GTGAAAACGGGGGCGAAGAAGTTGTCCATATTGGCCACGTTTAAA
TCAAAACTGGTGAAACTCACCCAGGGATTGGCTGAGACGAAAAAC
ATATTCTCAATAAACCCTTTAGGGAAATAGGCCAGGTTTTCACCG
TAACACGCCACATCTTGCGAATATATGTGTAGAAACTGCCGGAAA
TCGTCGTGGTATTCACTCCAGAGCGATGAAAACGTTTCAGTTTGC
TCATGGAAAACGGTGTAACAAGGGTGAACACTATCCCATATCACC
AGCTCACCGTCTTTCATTGCCATACGAAATTCCGGATGAGCATTC
ATCAGGCGGGCAAGAATGTGAATAAAGGCCGGATAAAACTTGTGC
TTATTTTTCTTTACGGTCTTTAAAAAGGCCGTAATATCCAGCTGA
ACGGTCTGGTTATAGGTACATTGAGCAACTGACTGAAATGCCTCA
AAATGTTCTTTACGATGCCATTGGGATATATCAACGGTGGTATAT
CCAGTGATTTTTTTCTCCATTTTAGCTTCCTTAGCTCCTGAAAAT
CTCGATAACTCAAAAAATACGCCCGGTAGTGATCTTATTTCATTA
TGGTGAAAGTTGGAACCTCTTACGTGCCGATCAACGTCTCATTTT
CGCCAGATATCGACGTCTAAGAAACCATTATTATCATGACATTAA
CCTATAAAAATAGGCGTATCACGAGGCCCTTTCGTCTTCACCTCG
AGAAGTCAGATCCTGTTATTAATGAAGTTAATGCTTCTCATTTTC
ATTGTAACCACAACCAGATGCAACCCCGAGTTGCAGATTGCGTTA
CCTCAAGAGTTGACATAGTGCGCGTTTGCTTTTAGGTTAGCGACC
GAAAATATAAATGATAATCATTATTAAAGCCTTTATCATTTTGTG
GAGGATGATAAAGAGGAGAAATACTAGATGCGTAAAGGCGAAGAG
CTGTTCACTGGTGTCGTCCCTATTCTGGTGGAACTGGATGGTGAT
GTCAACGGTCATAAGTTTTCCGTGCGTGGCGAGGGTGAAGGTGAC
GCAACTAATGGTAAACTGACGCTGAAGTTCATCTGTACTACTGGT
AAACTGCCGGTACCTTGGCCGACTCTGGTAACGACGCTGACTTAT
GGTGTTCAGTGCTTTGCTCGTTATCCGGACCATATGAAGCAGCAT
GACTTCTTCAAGTCCGCCATGCCGGAAGGCTATGTGCAGGAACGC
ACGATTTCCTTTAAGGATGACGGCACGTACAAAACGCGTGCGGAA
GTGAAATTTGAAGGCGATACCCTGGTAAACCGCATTGAGCTGAAA
GGCATTGACTTTAAAGAAGACGGCAATATCCTGGGCCATAAGCTG
GAATACAATTTTAACAGCCACAATGTTTACATCACCGCCGATAAA
CAAAAAAATGGCATTAAAGCGAATTTTAAAATTCGCCACAACGTG
GAGGATGGCAGCGTGCAGCTGGCTGATCACTACCAGCAAAACACT
CCAATCGGTGATGGTCCTGTTCTGCTGCCAGACAATCACTATCTG
AGCACGCAAAGCGTTCTGTCTAAAGATCCGAACGAGAAACGCGAT
CATATGGTTCTGCTGGAGTTCGTAACCGCAGCGGGCATCACGCAT
GGTATGGATGAACTGTACAAATGATGATAATAATCTAGACCAGGC
ATCAAATAAAACGAAAGGCTCAGTCGAAAGACTGGGCCTTTCGTT
TTATCTGTTGTTTGTCGGTGAACGCTCTCTACTAGAGTCACACTG
GCTCACCTTCGGGTGGGCCTTTCTGCGTTTATAGGTACCCGTGGA
TCCTCTAGAGTCGACCGAGCGGTATCAGCTCACTCAAAGGCGGTA
ATACGGTTATCCACAGAATCAGGGGATAACGCAGGAAAGAACATG
TGAGCAAAAGGCCAGCAAAAGGCCAGGAACCGTAAAAAGGCCGCG
TT

pBSI0-P_{abt}

(SEQ ID NO: 11)
GCTGGCGTTTTTCCATAGGCTCCGCCCCCCTGACGAGCATCACAA
AAATCGACGCTCAAGTCAGAGGTGGCGAAACCCGACAGGACTATA
AAGATACCAGGCGTTTCCCCCTGGAAGCTCCCTCGTGCGCTCTCC
TGTTCCGACCCTGCCGCTTACCGGATACCTGTCCGCCTTTCTCCC
TTCGGGAAGCGTGGCGCTTTCTCAATGCTCACGCTGTAGGTATCT
CAGTTCGGTGTAGGTCGTTCGCTCCAAGCTGGGCTGTGTGCACGA
ACCCCCCGTTCAGCCCGACCGCTGCGCCTTATCCGGTAACTATCG
TCTTGAGTCCAACCCGGTAAGACACGACTTATCGCCACTGGCAGC
AGCCACTGGTAACAGGATTAGCAGAGCGAGGTATGTAGGCGGTGC
TACAGAGTTCTTGAAGTGGTGGCCTAACTACGGCTACACTAGAAG
GACAGTATTTGGTATCTGCGCTCTGCTGAAGCCAGTTACCTTCGG
AAAAAGAGTTGGTAGCTCTTGATCCGGCAAACAAACCACCGCTGG
TAGCGGTGGTTTTTTTGTTTGCAAGCAGCAGATTACGCGCAGAAA
AAAAGGATCTCAAGAAGATCCTTTGATCTTTTCTACGGGGTCTGA
CGCTCAGTGGAACGAAAACTCACGTTAAGGGATTTTGGTCATGAC
TAGTGCTTGGATTCTCACCAATAAAAAACGCCCGGCGGCAACCGA
GCGTTCTGAACAAATCCAGATGGAGTTCTGAGGTCATTACTGGAT
CTATCAACAGGAGTCCAAGCGAGCTCGATATCAAATTACGCCCCG
CCCTGCCACTCATCGCAGTACTGTTGTAATTCATTAAGCATTCTG
CCGACATGGAAGCCATCACAGACGGCATGATGAACCTGAATCGCC
AGCGGCATCAGCACCTTGTCGCCTTGCGTATAATATTTGCCCATG
GTGAAAACGGGGGCGAAGAAGTTGTCCATATTGGCCACGTTTAAA
TCAAAACTGGTGAAACTCACCCAGGGATTGGCTGAGACGAAAAAC
ATATTCTCAATAAACCCTTTAGGGAAATAGGCCAGGTTTTCACCG
TAACACGCCACATCTTGCGAATATATGTGTAGAAACTGCCGGAAA
TCGTCGTGGTATTCACTCCAGAGCGATGAAAACGTTTCAGTTTGC
TCATGGAAAACGGTGTAACAAGGGTGAACACTATCCCATATCACC
AGCTCACCGTCTTTCATTGCCATACGAAATTCCGGATGAGCATTC
ATCAGGCGGGCAAGAATGTGAATAAAGGCCGGATAAAACTTGTGC
TTATTTTTCTTTACGGTCTTTAAAAAGGCCGTAATATCCAGCTGA
ACGGTCTGGTTATAGGTACATTGAGCAACTGACTGAAATGCCTCA
AAATGTTCTTTACGATGCCATTGGGATATATCAACGGTGGTATAT
CCAGTGATTTTTTTCTCCATTTTAGCTTCCTTAGCTCCTGAAAAT
CTCGATAACTCAAAAAATACGCCCGGTAGTGATCTTATTTCATTA
TGGTGAAAGTTGGAACCTCTTACGTGCCGATCAACGTCTCATTTT
CGCCAGATATCGACGTCTAAGAAACCATTATTATCATGACATTAA
CCTATAAAAATAGGCGTATCACGAGGCCCTTTCGTCTTCACCTCG
AGAACAGACCCGAAGAAAATGAAATATAAGAAAAGATCAACGAGT
GAAGAAAAAGTTCAAAAAATGGCTGCCGGGGAGGAAGGAAAGTAC
CGGATGGAAAGAGTCCCCCCTAAAGCAGACTGACAGACATAACAA
ATCCCCGGGGGGATTTGTGTATAAGAGACAGTACTTATCTGGAGG
TAATTGCAATATCTCTGTGAACTTACACAGGGTGGGCTTACCGCA
TACACTGACACTTAGCGGATCGACAGAACATTATTAACAGAGCAT
CACTGAACGCTACATAATCAGAGTTGCATAAATAAAATGTTATTA
ACATCACAATCACAACATTTCGAAAAGAGGAGAAATACTAGATGC
GTAAAGGCGAAGAGCTGTTCACTGGTGTCGTCCCTATTCTGGTGG
AACTGGATGGTGATGTCAACGGTCATAAGTTTTCCGTGCGTGGCG
AGGGTGAAGGTGACGCAACTAATGGTAAACTGACGCTGAAGTTCA
TCTGTACTACTGGTAAACTGCCGGTACCTTGGCCGACTCTGGTAA
CGACGCTGACTTATGGTGTTCAGTGCTTTGCTCGTTATCCGGACC
ATATGAAGCAGCATGACTTCTTCAAGTCCGCCATGCCGGAAGGCT
ATGTGCAGGAACGCACGATTTCCTTTAAGGATGACGGCACGTACA
AAACGCGTGCGGAAGTGAAATTTGAAGGCGATACCCTGGTAAACC
GCATTGAGCTGAAAGGCATTGACTTTAAAGAAGACGGCAATATCC
TGGGCCATAAGCTGGAATACAATTTTAACAGCCACAATGTTTACA
TCACCGCCGATAAACAAAAAAATGGCATTAAAGCGAATTTTAAAA
TTCGCCACAACGTGGAGGATGGCAGCGTGCAGCTGGCTGATCACT
ACCAGCAAAACACTCCAATCGGTGATGGTCCTGTTCTGCTGCCAG
ACAATCACTATCTGAGCACGCAAAGCGTTCTGTCTAAAGATCCGA
ACGAGAAACGCGATCATATGGTTCTGCTGGAGTTCGTAACCGCAG
CGGGCATCACGCATGGTATGGATGAACTGTACAAATGATGATAAT
AATCTAGACCAGGCATCAAATAAAACGAAAGGCTCAGTCGAAAGA
CTGGGCCTTTCGTTTTATCTGTTGTTTGTCGGTGAACGCTCTCTA
CTAGAGTCACACTGGCTCACCTTCGGGTGGGCCTTTCTGCGTTTA
TAGGTACCCGTGGATCCTCTAGAGTCGACCGAGCGGTATCAGCTC
ACTCAAAGGCGGTAATACGGTTATCCACAGAATCAGGGGATAACG
CAGGAAAGAACATGTGAGCAAAAGGCCAGCAAAAGGCCAGGAACC
GTAAAAAGGCCGCGTT

pBSI1

(SEQ ID NO: 12)
CGGGCTGAACGGGGGGTTCGTGCACACAGCCCAGCTTGGAGCGAA
CGACCTACACCGAACTGAGATACCTACAGCGTGAGCATTGAGAAA
GCGCCACGCTTCCCGAAGGGAGAAAGGCGGACAGGTATCCGGTAA
GCGGCAGGGTCGGAACAGGAGAGCGCACGAGGGAGCTTCCAGGGG
GAAACGCCTGGTATCTTTATAGTCCTGTCGGGTTTCGCCACCTCT
GACTTGAGCGTCGATTTTTGTGATGCTCGTCAGGGGGGCGGAGCC
TATGGAAAAACGCCAGCAACGCGGCCTTTTTACGGTTCCTGGCCT
TTTGCTGGCCTTTTGCTCACATGTTCTTTCCTGCGTTATCCCCTG
ATTCTGTGGATAACCGTATTACCGCCTTTGAGTGAGCTGATACCG
CTCGGTCGACTCTAGAGGATCCACGGGTACCTAACGGCAATAAAC
TGTTCACTTCAGTGATATTTAAAATATGCATCCTCTCCCTTTTTT
GTAAGTAATTATTATATCCGTGGGAGAGGAATACACATTGTCAGG
TAATCAATCATGCTGCAATAAATCATCGGCCAGTAAAGTGGAGAT
AGCCTCCATTCTCGAAAAATCCATACTCTCAGCGAAACCATCATC
AATCACTCATCCAGGCGTTTATGGGAGCGTCGCCAATGGCTGCTA
ACAATGCCAGACTTCCCCGTTGCGGAAATTCCACATCCCACAAAT
AGTCACAGTGATTGGGTGTTGAAATGATCCGGATGAGCATGTATC
TTTACGGTTATGTTATAACATAACAGGTAAAAATGAAAGAGGAGA
AATACTAGATGCGTAAAGGCGAAGAGCTGTTCACTGGTGTCGTCC
CTATTCTGGTGGAACTGGATGGTGATGTCAACGGTCATAAGTTTT
CCGTGCGTGGCGAGGGTGAAGGTGACGCAACTAATGGTAAACTGA
CGCTGAAGTTCATCTGTACTACTGGTAAACTGCCGGTACCTTGGC
CGACTCTGGTAACGACGCTGACTTATGGTGTTCAGTGCTTTGCTC
GTTATCCGGACCATATGAAGCAGCATGACTTCTTCAAGTCCGCCA
TGCCGGAAGGCTATGTGCAGGAACGCACGATTTCCTTTAAGGATG
ACGGCACGTACAAAACGCGTGCGGAAGTGAAATTTGAAGGCGATA
CCCTGGTAAACCGCATTGAGCTGAAAGGCATTGACTTTAAAGAAG
ACGGCAATATCCTGGGCCATAAGCTGGAATACAATTTTAACAGCC
ACAATGTTTACATCACCGCCGATAAACAAAAAAATGGCATTAAAG
CGAATTTTAAAATTCGCCACAACGTGGAGGATGGCAGCGTGCAGC
TGGCTGATCACTACCAGCAAAACACTCCAATCGGTGATGGTCCTG
TTCTGCTGCCAGACAATCACTATCTGAGCACGCAAAGCGTTCTGT
CTAAAGATCCGAACGAGAAACGCGATCATATGGTTCTGCTGGAGT
TCGTAACCGCAGCGGGCATCACGCATGGTATGGATGAACTGTACA
AATGATGATAATAATCTAGACCAGGCATCAAATAAAACGAAAGGC
TCAGTCGAAAGACTGGGCCTTTCGTTTTATCTGTTGTTTGTCGGT
GAACGCTCTCTACTAGAGTCACACTGGCTCACCTTCGGGTGGGCC
TTTCTGCGTTTATACTCGAGGTGAAGACGAAAGGGCCTCGTGATA
CGCCTATTTTTATAGGTTAATGTCATGATAATAATGGTTTCTTAG
ACGAGCGTTTTGTTATCAATAAAAAAGGCCCCCCGATTTGGGAGG
CCTTTTTAGTTAGAAAGCTTTAGCGAGGTTTCTTTTTCACCTGAA
TAGAGTGGTCATGATGGCACTGTTCAGGATGACGACACGCTTCTA
CTTCTACACATGCTGAACATAATCCATGTGCTTCAATAACATTAT
GCCGCAGGGCAAAACCCATTTTTGCCGCCAGCGTATGCATGATGT
CTTCCACGCCTTCAGCACATTCTTCTTTCACCGCGCCGCAGCGAT
CGCAAATAAACATGGCTGAAGTATGGGTGGGCTGATCGAACAGAT
GACAGAGCACATAACTGTTGGTGGATTCCACCTTATGCACAAAAC
CTTGTTCAAGCAGAAACTCCAGCGCGCGATAAACCGTTGGCGGCT
TGGCTTGCGGTTCAGCTTCGCGCAGCAAATCAAGCAGATCATAAG
CGCTGATAGCGCCATCCTGCAGACTCATCAGGCGCAACACTTCGA
GGCGCTGTGGGGTCAGGCGCACATTACGCTGTGCGCAGATTTTTT
CAGCCTGCGCTAATAACTCCTGCGTTGTGGTCTTTTCCATATATT
ATCTCCTGAATTCGCTAGCACAGTCCCTAGGACTGAGCTAGCTGT
AAATCGATATCTGGCGAAAATGAGACGTTGATCGGCACGTAAGAG
GTTCCAACTTTCACCATAATGAAATAAGATCACTACCGGGCGTAT
TTTTTGAGTTATCGAGATTTTCAGGAGCTAAGGAAGCTAAAATGG
AGAAAAAAATCACTGGATATACCACCGTTGATATATCCCAATGGC
ATCGTAAAGAACATTTTGAGGCATTTCAGTCAGTTGCTCAATGTA
CCTATAACCAGACCGTTCAGCTGGATATTACGGCCTTTTTAAAGA
CCGTAAAGAAAAATAAGCACAAGTTTTATCCGGCCTTTATTCACA
TTCTTGCCCGCCTGATGAATGCTCATCCGGAATTTCGTATGGCAA
TGAAAGACGGTGAGCTGGTGATATGGGATAGTGTTCACCCTTGTT
ACACCGTTTTCCATGAGCAAACTGAAACGTTTTCATCGCTCTGGA
GTGAATACCACGACGATTTCCGGCAGTTTCTACACATATATTCGC
AAGATGTGGCGTGTTACGGTGAAAACCTGGCCTATTTCCCTAAAG
GGTTTATTGAGAATATGTTTTTCGTCTCAGCCAATCCCTGGGTGA
GTTTCACCAGTTTTGATTTAAACGTGGCCAATATGGACAACTTCT
TCGCCCCCGTTTTCACCATGGGCAAATATTATACGCAAGGCGACA
AGGTGCTGATGCCGCTGGCGATTCAGGTTCATCATGCCGTCTGTG
ATGGCTTCCATGTCGGCAGAATGCTTAATGAATTACAACAGTACT
GCGATGAGTGGCAGGGCGGGGCGTAATTTGATATCGAGCTCGCTT
GGACTCCTGTTGATAGATCCAGTAATGACCTCAGAACTCCATCTG
GATTTGTTCAGAACGCTCGGTTGCCGCCGGGCGTTTTTTATTGGT
GAGAATCCAAGCACTAGTCATGACCAAAATCCCTTAACGTGAGTT
TTCGTTCCACTGAGCGTCAGACCCCGTAGAAAAGATCAAAGGATC
TTCTTGAGATCCTTTTTTTCTGCGCGTAATCTGCTGCTTGCAAAC
AAAAAAACCACCGCTACCAGCGGTGGTTTGTTTGCCGGATCAAGA
GCTACCAACTCTTTTTCCGAAGGTAACTGGCTTCAGCAGAGCGCA
GATACCAAATACTGTCCTTCTAGTGTAGCCGTAGTTAGGCCACCA
CTTCAAGAACTCTGTAGCACCGCCTACATACCTCGCTCTGCTAAT
CCTGTTACCAGTGGCTGCTGCCAGTGGCGATAAGTCGTGTCTTAC
CGGGTTGGACTCAAGACGATAGTTACCGGATAAGGCGCAGCGGT

pBSIM1

(SEQ ID NO: 13)
CGGGCTGAACGGGGGGTTCGTGCACACAGCCCAGCTTGGAGCGAA
CGACCTACACCGAACTGAGATACCTACAGCGTGAGCATTGAGAAA
GCGCCACGCTTCCCGAAGGGAGAAAGGCGGACAGGTATCCGGTAA
GCGGCAGGGTCGGAACAGGAGAGCGCACGAGGGAGCTTCCAGGGG
GAAACGCCTGGTATCTTTATAGTCCTGTCGGGTTTCGCCACCTCT
GACTTGAGCGTCGATTTTTGTGATGCTCGTCAGGGGGGCGGAGCC
TATGGAAAAACGCCAGCAACGCGGCCTTTTTACGGTTCCTGGCCT
TTTGCTGGCCTTTTGCTCACATGTTCTTTCCTGCGTTATCCCCTG
ATTCTGTGGATAACCGTATTACCGCCTTTGAGTGAGCTGATACCG
CTCGGTCGACTCTAGAGGATCCACGGGTACCTAACGGCAATAAAC
TGTTCACTTCAGTGATATTTAAAATATGCATCCTCTCCCTTTTTT
GTAAGTAATTATTATATCCGTGGGAGAGGAATACACATTGTCAGG
TAATCAATCATGCTGCAATAAATCATCGGCCAGTAAAGTGGAGAT
AGCCTCCATTCTCGAAAAATCCATACTCTCAGCGAAACCATCATC
AATCACTCATCCAGGCGTTTATGGGAGCGTCGCCAATGGCTGCTA
ACAATGCCAGACTTCCCCGTTGCGGAAATTCCACATCCCACAAAT
AGTCACAGTGATTGGGTGTTGAAATGATCCGGATGAGCATGTATC
TTTACGGTTATGTTATAACATAACAGGTAAAGGCTAGCAAAACAA
TAACTAGGATTCGAATGAAAGTTGCCGTTTATTGTCGTGTTAGCA
CCCTGGAACAGAAAGAACATGGTCATAGCATTGAAGAACAAGAGC
GTAAACTGAAAAGCTTCTGCGATATTAATGATTGGACCGTGTATG
ATACCTATATCGATGCAGGTTATAGCGGTGCAAAACGTGATCGTC
CGGAACTGCAGCGTCTGATGAATGATATTAACAAATTTGATCTGG
TGCTGGTGTATAAACTGGATCGTCTGACCCGTAATGTTCGTGATC
TGCTGGACCTGCTGGAAATCTTTGAAAAAAATGATGTGAGCTTTC
GTAGCGCCACCGAAGTTTATGATACCACCACCGCAATGGGTCGTC
TGTTTGTTACCCTGGTTGGTGCAATGGCAGAATGGGAACGTGAAA
CCATTCGTGAACGTACCCAGATGGGTAAACTGGCAGCACTGCGTA
AAGGTATTATGCTGACCACCCCTCCGTTTTATTATGACCGTGTGG
ATAATAAGTTTGTGCCGAACAAATACAAAGACGTTATTCTGTGGG
CATATGACGAAGCAATGAAAGGTCAGAGCGCAAAAGCAATTGCAC
GCAAACTGAATAATAGCGATATTCCGCCTCCGAATAATACCCAGT
GGCAGGGTCGTACCATTACCCATGCCCTGCGTAATCCGTTTACCC
GTGGTCATTTTGATTGGGGTGGTGTGCATATTGAAAATAACCATG
AACCGATCATCACCGATGAGATGTATGAGAAAGTTAAAGATCGCC
TGAATGAACGCGTGAACACCAAAAAAGTTCGTCATACCAGCATTT
TTCGTGGCAAACTGGTTTGTCCGGTTTGTAATGCACGCCTGACCC
TGAATAGCCATAAAAAGAAAAGCAATAGCGGCTATATCTTTGTGA
AACAGTACTACTGCAACAACTGTAAAGTTACCCCGAATCTGAAAC
CGGTGTACATCAAAGAAAAAGAAGTGATTAAAGTTTTTTACAATT
ATCTGAAACGCTTCGATCTGGAAAAATATGAGGTTACCCAGAAAC
AGAACGAACCGGAAATCACCATCGATATCAATAAAGTTATGGAAC
AGCGCAAACGCTACCATAAACTGTATGCAAGCGGTCTGATGCAAG
AAGATGAACTGTTTGACCTGATTAAAGAAACCGATCAGACCATTG
CCGAATATGAAAAACAGAATGAAAACCGCGAAGTGAAGCAGTATG
ATATCGAAGATATCAAACAGTATAAAGATCTGCTGTTAGAAATGT
GGGATATCAGCTCCGATGAAGATAAAGAGGACTTTATCAAAATGG
CGATTAAAAACATCTATTTTGAATATATCATTGGCACCGGTAACA
CCAGCCGTAAACGTAATAGCCTGAAAATTACGAGCATTGAATTCT
ATTAACAGATAAAAAAAATCCTTAGCTTTCGCTAAGGATGATTAA
TAATCTAGACCAGGCATCAAATAAAACGAAAGGCTCAGTCGAAAG
ACTGGGCCTTTCGTTTTATCTGTTGTTTGTCGGTGAACGCTCTCT
ACTAGAGTCACACTGGCTCACCTTCGGGTGGGCCTTTCTGCGTTT
ATACTCGAGGTGAAGACGAAAGGGCCTCGTGATACGCCTATTTTT
ATAGGTTAATGTCATGATAATAATGGTTTCTTAGACGAGCGTTTT
GTTATCAATAAAAAAGGCCCCCCGATTTGGGAGGCCTTTTTAGTT
AGAAAGCTTTAGCGAGGTTTCTTTTTCACCTGAATAGAGTGGTCA
TGATGGCACTGTTCAGGATGACGACACGCTTCTACTTCTACACAT
GCTGAACATAATCCATGTGCTTCAATAACATTATGCCGCAGGGCA
AAACCCATTTTTGCCGCCAGCGTATGCATGATGTCTTCCACGCCT
TCAGCACATTCTTCTTTCACCGCGCCGCAGCGATCGCAAATAAAC
ATGGCTGAAGTATGGGTGGGCTGATCGAACAGATGACAGAGCACA
TAACTGTTGGTGGATTCCACCTTATGCACAAAACCTTGTTCAAGC
AGAAACTCCAGCGCGCGATAAACCGTTGGCGGCTTGGCTTGCGGT
TCAGCTTCGCGCAGCAAATCAAGCAGATCATAAGCGCTGATAGCG
CCATCCTGCAGACTCATCAGGCGCAACACTTCGAGGCGCTGTGGG
GTCAGGCGCACATTACGCTGTGCGCAGATTTTTTCAGCCTGCGCT
AATAACTCCTGCGTTGTGGTCTTTTCCATATATTATCTCCTGAAT
TCGCTAGCACAGTCCCTAGGACTGAGCTAGCTGTAAATCGATATC
TGGCGAAAATGAGACGTTGATCGGCACGTAAGAGGTTCCAACTTT
CACCATAATGAAATAAGATCACTACCGGGCGTATTTTTTGAGTTA
TCGAGATTTTCAGGAGCTAAGGAAGCTAAAATGGAGAAAAAAATC
ACTGGATATACCACCGTTGATATATCCCAATGGCATCGTAAAGAA
CATTTTGAGGCATTTCAGTCAGTTGCTCAATGTACCTATAACCAG
ACCGTTCAGCTGGATATTACGGCCTTTTTAAAGACCGTAAAGAAA
AATAAGCACAAGTTTTATCCGGCCTTTATTCACATTCTTGCCCGC
CTGATGAATGCTCATCCGGAATTTCGTATGGCAATGAAAGACGGT
GAGCTGGTGATATGGGATAGTGTTCACCCTTGTTACACCGTTTTC
CATGAGCAAACTGAAACGTTTTCATCGCTCTGGAGTGAATACCAC
GACGATTTCCGGCAGTTTCTACACATATATTCGCAAGATGTGGCG
TGTTACGGTGAAAACCTGGCCTATTTCCCTAAAGGGTTTATTGAG
AATATGTTTTTCGTCTCAGCCAATCCCTGGGTGAGTTTCACCAGT
TTTGATTTAAACGTGGCCAATATGGACAACTTCTTCGCCCCCGTT
TTCACCATGGGCAAATATTATACGCAAGGCGACAAGGTGCTGATG
CCGCTGGCGATTCAGGTTCATCATGCCGTCTGTGATGGCTTCCAT
GTCGGCAGAATGCTTAATGAATTACAACAGTACTGCGATGAGTGG
CAGGGCGGGGCGTAATTTGATATCGAGCTCGCTTGGACTCCTGTT
GATAGATCCAGTAATGACCTCAGAACTCCATCTGGATTTGTTCAG
AACGCTCGGTTGCCGCCGGGCGTTTTTTATTGGTGAGAATCCAAG
CACTAGTCATGACCAAAATCCCTTAACGTGAGTTTTCGTTCCACT
GAGCGTCAGACCCCGTAGAAAAGATCAAAGGATCTTCTTGAGATC
CTTTTTTTCTGCGCGTAATCTGCTGCTTGCAAACAAAAAAACCAC
CGCTACCAGCGGTGGTTTGTTTGCCGGATCAAGAGCTACCAACTC
TTTTTCCGAAGGTAACTGGCTTCAGCAGAGCGCAGATACCAAATA
CTGTCCTTCTAGTGTAGCCGTAGTTAGGCCACCACTTCAAGAACT
CTGTAGCACCGCCTACATACCTCGCTCTGCTAATCCTGTTACCAG
TGGCTGCTGCCAGTGGCGATAAGTCGTGTCTTACCGGGTTGGACT
CAAGACGATAGTTACCGGATAAGGCGCAGCGGT

pBSIM2

(SEQ ID NO: 14)
AGGTAGAGACCCACGAGGCAGACCTCAGCGCTAGCGGAGTGTATA
CTGGCTTACTATGTTGGCACTGATGAGGGTGTCAGTGAAGTGCTT
CATGTGGCAGGAGAAAAAAGGCTGCACCGGTGCGTCAGCAGAATA
TGTGATACAGGATATATTCCGCTTCCTCGCTCACTGACTCGCTAC
GCTCGGTCGTTCGACTGCGGCGAGCGGAAATGGCTTACGAACGGG
GCGGAGATTTCCTGGAAGATGCCAGGAAGATACTTAACAGGGAAG
TGAGAGGGCCGCGGCAAAGCCGTTTTTCCATAGGCTCCGCCCCCC
TGACAAGCATCACGAAATCTGACGCTCAAATCAGTGGTGGCGAAA
CCCGACAGGACTATAAAGATACCAGGCGTTTCCCCCTGGCGGCTC
CCTCGTGCGCTCTCCTGTTCCTGCCTTTCGGTTTACCGGTGTCAT
TCCGCTGTTATGGCCGCGTTTGTCTCATTCCACGCCTGACACTCA
GTTCCGGGTAGGCAGTTCGCTCCAAGCTGGACTGTATGCACGAAC
CCCCCGTTCAGTCCGACCGCTGCGCCTTATCCGGTAACTATCGTC
TTGAGTCCAACCCGGAAAGACATGCAAAAGCACCACTGGCAGCAG
CCACTGGTAATTGATTTAGAGGAGTTAGTCTTGAAGTCATGCGCC
GGTTAAGGCTAAACTGAAAGGACAAGTTTTGGTGACTGCGCTCCT
CCAAGCCAGTTACCTCGGTTCAAAGAGTTGGTAGCTCAGAGAACC
TTCGAAAAACCGCCCTGCAAGGCGGTTTTTTCGTTTTCAGAGCAA
GAGATTACGCGCAGACCAAAACGATCTCAAGAAGATCATCTTATT
AAGGGGTCTGACGCTCAGTGGAACGAAAAGTATATATGAGTAAAC
TTGGTCTGACAGCTCGAGCTTACCCGTCTTACTGTCCCTAGTGCT
TGGATTCTCACCAATAAAAAACGCCCGGCGGCAACCGAGCGTTCT
GAACAAATCCAGATGGAGTTCTGAGGTCATTACTGGATCTATCAA
CAGGAGTCCAAGCGAGCTCGTAAACTTGGTCTGACAGTTACCAAT
GCTTAATCAGTGAGGCACCTATCTCAGCGATCTGTCTATTTCGTT
CATCCATAGTTGCCTGACTCCCCGTCGTGTAGATAACTACGATAC
GGGAGGGCTTACCATCTGGCCCCAGTGCTGCAATGATACCGCGTG
ATCCACGCTCACCGGCTCCAGATTTATCAGCAATAAACCAGCCAG
CCGGAAGGGCCGAGCGCAGAAGTGGTCCTGCAACTTTATCCGCCT
CCATCCAGTCTATTAATTGTTGCCGGGAAGCTAGAGTAAGTAGTT
CGCCAGTTAATAGTTTGCGCAACGTTGTTGCCATTGCTACAGGCA
TCGTGGTGTCACGCTCGTCGTTTGGTATGGCTTCATTCAGCTCCG
GTTCCCAACGATCAAGGCGAGTTACATGATCCCCCATGTTGTGCA
AAAAATCGGTTAGCTCCTTCGGTCCTCCGATCGTTGTCAGAAGTA
AGTTGGCCGCAGTGTTATCACTCATGGTTATGGCAGCACTGCATA
ATTCTCTTACTGTCATGCCATCCGTAAGATGCTTTTCTGTGACTG
GTGAGTACTCAACCAAGTCATTCTGAGAATAGTGTATGCGGCGAC
CGAGTTGCTCTTGCCCGGCGTCAATACGGGATAATACCGCGCCAC
ATAGCAGAACTTTAAAAGTGCTCATCATTGGAAAACGTTCTTCGG
GGCGAAAACTCTCAAGGATCTTACCGCTGTTGAGATCCAGTTCGA
TGTAACCCACTCGTGCACCCAACTGATCTTCAGCATCTTTTACTT
TCACCAGCGTTTCTGGGTGAGCAAAAACAGGAAGGCAAAATGCCG
CAAAAAAGGGAATAAGGGCGACACGGAAATGTTGAATACTCATAC
TCTTCCTTTTTCAATATTATTGAAGCATTTATCAGGGTTATTGTC
TCATGAGCGGATACATATTTGAATGTTACGTGCCCGATCAACTGC
GTTTATATACTAGTAGCGGCAGCTGCAGTCCGGCAAAAAAGGGCA
AGGTGTCACCACCCTGCCCTTTTTCTTTAAAACCGAAAAGATTAC
TTCGCGTTATGCAGGCTTCCTCGCTCACTGACTCGCTGCGCTCGG
TCGTTCGGCTGCGGCGAGCGGTATCAGCTCACTCAAAGGCGGTAA
TTTGACAGCTAGCTCAGTCCTAGGTATAATGCTAGCAGTTCGATG
AGAGCGATAACCAATCATCAGATAACTATGGCGGCACGTGCATTA
ACCACGGTTGTATCCCGTCTAAAGTACTCGTTCATCATTTGTACA
GTTCATCCATACCATGCGTGATGCCCGCTGCGGTTACGAACTCCA
GCAGAACCATATGATCGCGTTTCTCGTTCGGATCTTTAGACAGAA
CGCTTTGCGTGCTCAGATAGTGATTGTCTGGCAGCAGAACAGGAC
CATCACCGATTGGAGTGTTTTGCTGGTAGTGATCAGCCAGCTGCA
CGCTGCCATCCTCCACGTTGTGGCGAATTTTAAAATTCGCTTTAA
TGCCATTTTTTTGTTTATCGGCGGTGATGTAAACATTGTGGCTGT
TAAAATTGTATTCCAGCTTATGGCCCAGGATATTGCCGTCTTCTT
TAAAGTCAATGCCTTTCAGCTCAATGCGGTTTACCAGGGTATCGC
CTTCAAATTTCACTTCCGCACGCGTTTTGTACGTGCCGTCATCCT
TAAAGGAAATCGTGCGTTCCTGCACATAGCCTTCCGGCATGGCGG
ACTTGAAGAAGTCATGCTGCTTCATATGGTCCGGATAACGAGCAA
AGCACTGAACACCATAAGTCAGCGTCGTTACCAGAGTCGGCCAAG
GTACCGGCAGTTTACCAGTAGTACAGATGAACTTCAGCGTCAGTT
TACCATTAGTTGCGTCACCTTCACCCTCGCCACGCACGGAAAACT
TATGACCGTTGACATCACCATCCAGTTCCACCAGAATAGGGACGA
CACCAGTGAACAGCTCTTCGCCTTTACGCATCTAGTATTTCTCCT
CTTTCTCTAGATTAAACAAAATTATTTGTAGAGGCTGTTTCGTCC
TCACGGACTCATCAGACCGGAAAGCACATCCGGTGACAGCTATTT
TATTATGGAAGTTTGTTCACTCAACATTGCAAGACTGTACATACT
TCCATAGTTTATTAATATAAACGCAGAAAGGCCCACCCGAAGGTG
AGCCAGTGTGACTCTAGTAGAGAGCGTTCACCGACAAACAACAGA
TAAAACGAAAGGCCCAGTCTTTCGACTGAGCCTTTCGTTTTATTT
GATGCCTGGCTAGGGCCCATACCCAAAAAAAAACCCCGCCCCTGA
CAGGGCGGGGTTTTTTTT

pBSIM2-bfmo

(SEQ ID NO: 15)
AGGTAGAGACCCACGAGGCAGACCTCAGCGCTAGCGGAGTGTATA
CTGGCTTACTATGTTGGCACTGATGAGGGTGTCAGTGAAGTGCTT
CATGTGGCAGGAGAAAAAAGGCTGCACCGGTGCGTCAGCAGAATA
TGTGATACAGGATATATTCCGCTTCCTCGCTCACTGACTCGCTAC
GCTCGGTCGTTCGACTGCGGCGAGCGGAAATGGCTTACGAACGGG
GCGGAGATTTCCTGGAAGATGCCAGGAAGATACTTAACAGGGAAG
TGAGAGGGCCGCGGCAAAGCCGTTTTTCCATAGGCTCCGCCCCCC
TGACAAGCATCACGAAATCTGACGCTCAAATCAGTGGTGGCGAAA
CCCGACAGGACTATAAAGATACCAGGCGTTTCCCCCTGGCGGCTC
CCTCGTGCGCTCTCCTGTTCCTGCCTTTCGGTTTACCGGTGTCAT
TCCGCTGTTATGGCCGCGTTTGTCTCATTCCACGCCTGACACTCA
GTTCCGGGTAGGCAGTTCGCTCCAAGCTGGACTGTATGCACGAAC
CCCCCGTTCAGTCCGACCGCTGCGCCTTATCCGGTAACTATCGTC
TTGAGTCCAACCCGGAAAGACATGCAAAAGCACCACTGGCAGCAG
CCACTGGTAATTGATTTAGAGGAGTTAGTCTTGAAGTCATGCGCC
GGTTAAGGCTAAACTGAAAGGACAAGTTTTGGTGACTGCGCTCCT
CCAAGCCAGTTACCTCGGTTCAAAGAGTTGGTAGCTCAGAGAACC
TTCGAAAAACCGCCCTGCAAGGCGGTTTTTTCGTTTTCAGAGCAA
GAGATTACGCGCAGACCAAAACGATCTCAAGAAGATCATCTTATT
AAGGGGTCTGACGCTCAGTGGAACGAAAAGTATATATGAGTAAAC
TTGGTCTGACAGCTCGAGCTTACCCGTCTTACTGTCCCTAGTGCT
TGGATTCTCACCAATAAAAAACGCCCGGCGGCAACCGAGCGTTCT
GAACAAATCCAGATGGAGTTCTGAGGTCATTACTGGATCTATCAA
CAGGAGTCCAAGCGAGCTCGTAAACTTGGTCTGACAGTTACCAAT
GCTTAATCAGTGAGGCACCTATCTCAGCGATCTGTCTATTTCGTT
CATCCATAGTTGCCTGACTCCCCGTCGTGTAGATAACTACGATAC
GGGAGGGCTTACCATCTGGCCCCAGTGCTGCAATGATACCGCGTG
ATCCACGCTCACCGGCTCCAGATTTATCAGCAATAAACCAGCCAG
CCGGAAGGGCCGAGCGCAGAAGTGGTCCTGCAACTTTATCCGCCT
CCATCCAGTCTATTAATTGTTGCCGGGAAGCTAGAGTAAGTAGTT
CGCCAGTTAATAGTTTGCGCAACGTTGTTGCCATTGCTACAGGCA
TCGTGGTGTCACGCTCGTCGTTTGGTATGGCTTCATTCAGCTCCG
GTTCCCAACGATCAAGGCGAGTTACATGATCCCCCATGTTGTGCA
AAAAATCGGTTAGCTCCTTCGGTCCTCCGATCGTTGTCAGAAGTA
AGTTGGCCGCAGTGTTATCACTCATGGTTATGGCAGCACTGCATA
ATTCTCTTACTGTCATGCCATCCGTAAGATGCTTTTCTGTGACTG
GTGAGTACTCAACCAAGTCATTCTGAGAATAGTGTATGCGGCGAC
CGAGTTGCTCTTGCCCGGCGTCAATACGGGATAATACCGCGCCAC
ATAGCAGAACTTTAAAAGTGCTCATCATTGGAAAACGTTCTTCGG
GGCGAAAACTCTCAAGGATCTTACCGCTGTTGAGATCCAGTTCGA
TGTAACCCACTCGTGCACCCAACTGATCTTCAGCATCTTTTACTT
TCACCAGCGTTTCTGGGTGAGCAAAAACAGGAAGGCAAAATGCCG
CAAAAAAGGGAATAAGGGCGACACGGAAATGTTGAATACTCATAC
TCTTCCTTTTTCAATATTATTGAAGCATTTATCAGGGTTATTGTC
TCATGAGCGGATACATATTTGAATGTTACGTGCCCGATCAACTGC
GTTTATATACTAGTAGCGGCAGCTGCAGTCCGGCAAAAAAGGGCA
AGGTGTCACCACCCTGCCCTTTTTCTTTAAAACCGAAAAGATTAC
TTCGCGTTATGCAGGCTTCCTCGCTCACTGACTCGCTGCGCTCGG
TCGTTCGGCTGCGGCGAGCGGTATCAGCTCACTCAAAGGCGGTAA
TTTGACAGCTAGCTCAGTCCTAGGTATAATGCTAGCAGTTCGATG
AGAGCGATAACCAATCATCAGATAACTATGGCGGCACGTGCATTA
ACCACGGTTGTATCCCGTCTAAAGTACTCGTTTAGGCCTCTTTTG
CCACCGGGATTTCGGACTTGTCGCTCAGGTAAGCCTCCAGGCTGT
CGTCCAGAGCATCGATCCACGGGGTATGGTGCTTTGGAGCCATCG
TGCCGGTCATCAGGGAGCGATAGCTGTGGTCGCGAAACGTCATGA
TGTTCTCTTTCTTATGATGTTTCCATTCCAGGAACGTTTTGTTCG
TCGCCGGAATATCAAAGCTCGGATAATCGGTCATGTCGATCAGGT
TCTGGATATAGTCACCCTGGTAGGTATACATCTCTTCAGCCGTGA
CCAGCGTCAGCTCTTTCTCGCGCCACGCCATGCTGTCGGCCTTCA
TTTCTTCTTTGCTCGGCAACGGCAAACGGCCCATGATCACATCAC
GCGCATACCAAGCTTGGGCATCGAACATGTTGAAGCTGTACCATT
GGTCTTGCATACCAATGTAGAAGAACTTCGGATTATCCTCCCAAA
CAACGCCCTTGTACAGGTTCAGCGGCCACAGGCGATTGTTGGTCA
CCAGGCGCAAGTCGTCATTCAGAAACGGGAAGTGGTGGATGTAAC
CGGTGCACAGGATGATGGCATCAACTTTCTCCGAAGAACCATCTG
CGAAGTAAGCATTCTCGGTATCCACACGCACCAGGTTCGGACGCT
CGTCCCAGTTCTCCGGCCATTTGTAACCCATCGGTGCGGTGCGAT
AGCAGCTAATCAGTTTCTTCGCGCCATACTTGTAGCACTGGCTGC
CAATATCTTCCGCAGAGTACGAGCTGCCCACCAACAGAACGGTCT
TATCCTTGAACTCCAGCGCATCACGAAAGTCGTGGGCATGCAGAA
TACGACCACCGAACTTTTCGAAGCCTTCGAACTCCGGGACATACG
GGGTGCTGAAGTGACCGGTACAGCAAACCACATAGTCAAATTCTT
CGCTATAGATGGTGTCAGTGGTATGGTCCTGAACCGTCACAGTAA
AGGTCTGAGAATCCTCATTGAACTCAACATGACGAACCGCGGTAT
TGAAACGGATGTATTTGCGGACACCCGCCTTTTCCACGCGACCCT
TGATATAGTCCCACAGCACTTCACGCGGTGGGTAGCTGGCAATTG
GCTTACCAAAGTGTTCATCAAAGGTGTAATCAGCAAACTCCAAAC
ATTCTTTCGGACCGTTGGACCACAGGTAACGGTACATGGAGCTGT
GGACCGGTTCGCCGTTTTCGTCCAGACCGGTACGCCAAGTATAGT
TCCACTGGCCACCCCAGTCCGCCTGTTTCTCAAAACAAACCAACT
CCGGGATCTCAGCGCCTTTCTCTTGTGCGCTTTGAAACGCACGCA
ATTGCGCCATACCCGATGGGCCTGCGCCCAGAATTGCAATACGGG
TCGCCATTTTTTACCTCCTAAAAGTCTCTAGATTAAACAAAATTA
TTTGTAGAGGCTGTTTCGTCCTCACGGACTCATCAGACCGGAAAG
CACATCCGGTGACAGCTATTTTATTATGGAAGTTTGTTCACTCAA
CATTGCAAGACTGTACATACTTCCATAGTTTATTAATATAAACGC
AGAAAGGCCCACCCGAAGGTGAGCCAGTGTGACTCTAGTAGAGAG
CGTTCACCGACAAACAACAGATAAAACGAAAGGCCCAGTCTTTCG
ACTGAGCCTTTCGTTTTATTTGATGCCTGGCTAGGGCCCATACCC
AAAAAAAAACCCCGCCCCTGACAGGGCGGGGTTTTTTTT

pBSIT0

(SEQ ID NO: 16)
CGGGCTGAACGGGGGGTTCGTGCACACAGCCCAGCTTGGAGCGAA
CGACCTACACCGAACTGAGATACCTACAGCGTGAGCATTGAGAAA
GCGCCACGCTTCCCGAAGGGAGAAAGGCGGACAGGTATCCGGTAA
GCGGCAGGGTCGGAACAGGAGAGCGCACGAGGGAGCTTCCAGGGG
GAAACGCCTGGTATCTTTATAGTCCTGTCGGGTTTCGCCACCTCT
GACTTGAGCGTCGATTTTTGTGATGCTCGTCAGGGGGGCGGAGCC
TATGGAAAAACGCCAGCAACGCGGCCTTTTTACGGTTCCTGGCCT
TTTGCTGGCCTTTTGCTCACATGTTCTTTCCTGCGTTATCCCCTG
ATTCTGTGGATAACCGTATTACCGCCTTTGAGTGAGCTGATACCG
CTCGGTCGACTCTAGAGGATCCACGGGTACCTAACGGCAATAAAC
TGTTCACTTCAGTGATATTTAAAATATGCATCCTCTCCCTTTTTT
GTAAGTAATTATTATATCCGTGGGAGAGGAATACACATTGTCAGG
TAATCAATCATGCTGCAATAAATCATCGGCCAGTAAAGTGGAGAT
AGCCTCCATTCTCGAAAAATCCATACTCTCAGCGAAACCATCATC
AATCACTCATCCAGGCGTTTATGGGAGCGTCGCCAATGGCTGCTA
ACAATGCCAGACTTCCCCGTTGCGGAAATTCCACATCCCACAAAT
AGTCACAGTGATTGGGTGTTGAAATGATCCGGATGAGCATGTATC
TTTACGGTTATGTTATAACATAACAGGTAAAGGCTAGCAAAACAA
TAACTAGGATTCGAGGAGAAAAACATGAAAAAAAGAGGGGCGTTT
TTAGGGCTGTTGTTGGTTTCTGCCTGCGCATCAGTTTTCGCTGCC
AATAATGAAACCAGCAAGTCGGTCACTTTCCCAAAGTGTGAAGGT
CTGGATGCTGCCGGAATTGCCGCGAGCGTAAAACGTGATTACCAA
CAAAATCGCGTGGCGCGCTGGGCTGATGATCAAAAAATTGTCGGT
CAGGCCGATCCCGTGGCATGGGTCAGTTTGCAGGACATTCAGGGG
AAAGATGATAAATGGTCAGTACCGCTAACCGTGCGTGGTAAAAGT
GCCGATATTCATTACCAGGTCAGCGTGGACTGCAAAGCGGGAATG
GCGGAATATCAGCGGCGTTAACAGATAAAAAAAATCCTTAGCTTT
CGCTAAGGATGATTAATAATCTAGACCAGGCATCAAATAAAACGA
AAGGCTCAGTCGAAAGACTGGGCCTTTCGTTTTATCTGTTGTTTG
TCGGTGAACGCTCTCTACTAGAGTCACACTGGCTCACCTTCGGGG
GGCCTTTCTGCGTTTATACTCGAGGTGAAGACGAAAGGGCCTCGT
GATACGCCTATTTTTATAGGTTAATGTCATGATAATAATGGTTTC
TTAGACGAGCGTTTTGTTATCAATAAAAAAGGCCCCCCGATTTGG
GAGGCCTTTTTAGTTAGAAAGCTTTAGCGAGGTTTCTTTTTCACC
TGAATAGAGTGGTCATGATGGCACTGTTCAGGATGACGACACGCT
TCTACTTCTACACATGCTGAACATAATCCATGTGCTTCAATAACA
TTATGCCGCAGGGCAAAACCCATTTTTGCCGCCAGCGTATGCATG
ATGTCTTCCACGCCTTCAGCACATTCTTCTTTCACCGCGCCGCAG
CGATCGCAAATAAACATGGCTGAAGTATGGGTGGGCTGATCGAAC
AGATGACAGAGCACATAACTGTTGGTGGATTCCACCTTATGCACA
AAACCTTGTTCAAGCAGAAACTCCAGCGCGCGATAAACCGTTGGC
GGCTTGGCTTGCGGTTCAGCTTCGCGCAGCAAATCAAGCAGATCA
TAAGCGCTGATAGCGCCATCCTGCAGACTCATCAGGCGCAACACT
TCGAGGCGCTGTGGGGTCAGGCGCACATTACGCTGTGCGCAGATT
TTTTCAGCCTGCGCTAATAACTCCTGCGTTGTGGTCTTTTCCATA
TATTATCTCCTGAATTCGCTAGCACAGTCCCTAGGACTGAGCTAG
CTGTAAATCGATATCTGGCGAAAATGAGACGTTGATCGGCACGTA
AGAGGTTCCAACTTTCACCATAATGAAATAAGATCACTACCGGGC
GTATTTTTTGAGTTATCGAGATTTTCAGGAGCTAAGGAAGCTAAA
ATGGAGAAAAAAATCACTGGATATACCACCGTTGATATATCCCAA
TGGCATCGTAAAGAACATTTTGAGGCATTTCAGTCAGTTGCTCAA
TGTACCTATAACCAGACCGTTCAGCTGGATATTACGGCCTTTTTA
AAGACCGTAAAGAAAAATAAGCACAAGTTTTATCCGGCCTTTATT
CACATTCTTGCCCGCCTGATGAATGCTCATCCGGAATTTCGTATG
GCAATGAAAGACGGTGAGCTGGTGATATGGGATAGTGTTCACCCT
TGTTACACCGTTTTCCATGAGCAAACTGAAACGTTTTCATCGCTC
TGGAGTGAATACCACGACGATTTCCGGCAGTTTCTACACATATAT
TCGCAAGATGTGGCGTGTTACGGTGAAAACCTGGCCTATTTCCCT
AAAGGGTTTATTGAGAATATGTTTTTCGTCTCAGCCAATCCCTGG
GTGAGTTTCACCAGTTTTGATTTAAACGTGGCCAATATGGACAAC
TTCTTCGCCCCCGTTTTCACCATGGGCAAATATTATACGCAAGGC
GACAAGGTGCTGATGCCGCTGGCGATTCAGGTTCATCATGCCGTC
TGTGATGGCTTCCATGTCGGCAGAATGCTTAATGAATTACAACAG
TACTGCGATGAGTGGCAGGGCGGGGCGTAATTTGATATCGAGCTC
GCTTGGACTCCTGTTGATAGATCCAGTAATGACCTCAGAACTCCA
TCTGGATTTGTTCAGAACGCTCGGTTGCCGCCGGGCGTTTTTTAT
TGGTGAGAATCCAAGCACTAGTCATGACCAAAATCCCTTAACGTG
AGTTTTCGTTCCACTGAGCGTCAGACCCCGTAGAAAAGATCAAAG
GATCTTCTTGAGATCCTTTTTTTCTGCGCGTAATCTGCTGCTTGC
AAACAAAAAAACCACCGCTACCAGCGGTGGTTTGTTTGCCGGATC
AAGAGCTACCAACTCTTTTTCCGAAGGTAACTGGCTTCAGCAGAG
CGCAGATACCAAATACTGTCCTTCTAGTGTAGCCGTAGTTAGGCC
ACCACTTCAAGAACTCTGTAGCACCGCCTACATACCTCGCTCTGC
TAATCCTGTTACCAGTGGCTGCTGCCAGTGGCGATAAGTCGTGTC
TTACCGGGTTGGACTCAAGACGATAGTTACCGGATAAGGCGCAGC
GGT

PBSIT1

(SEQ ID NO: 17)
CGGGCTGAACGGGGGGTTCGTGCACACAGCCCAGCTTGGAGCGAA
CGACCTACACCGAACTGAGATACCTACAGCGTGAGCATTGAGAAA
GCGCCACGCTTCCCGAAGGGAGAAAGGCGGACAGGTATCCGGTAA
GCGGCAGGGTCGGAACAGGAGAGCGCACGAGGGAGCTTCCAGGGG
GAAACGCCTGGTATCTTTATAGTCCTGTCGGGTTTCGCCACCTCT
GACTTGAGCGTCGATTTTTGTGATGCTCGTCAGGGGGGCGGAGCC
TATGGAAAAACGCCAGCAACGCGGCCTTTTTACGGTTCCTGGCCT
TTTGCTGGCCTTTTGCTCACATGTTCTTTCCTGCGTTATCCCCTG
ATTCTGTGGATAACCGTATTACCGCCTTTGAGTGAGCTGATACCG
CTCGGTCGACTCTAGAGGATCCACGGGTACCTAACGGCAATAAAC
TGTTCACTTCAGTGATATTTAAAATATGCATCCTCTCCCTTTTTT
GTAAGTAATTATTATATCCGTGGGAGAGGAATACACATTGTCAGG
TAATCAATCATGCTGCAATAAATCATCGGCCAGTAAAGTGGAGAT
AGCCTCCATTCTCGAAAAATCCATACTCTCAGCGAAACCATCATC
AATCACTCATCCAGGCGTTTATGGGAGCGTCGCCAATGGCTGCTA
ACAATGCCAGACTTCCCCGTTGCGGAAATTCCACATCCCACAAAT
AGTCACAGTGATTGGGTGTTGAAATGATCCGGATGAGCATGTATC
TTTACGGTTATGTTATAACATAACAGGTAAAGGCTAGCAAAACAA
TAACTAGGATTCGAGGAGAAAAACATGAAAAAAAGAGGGGCGTTT
TTAGGGCTGTTGTTGGTTTCTGCCTGCGCATCAGTTTTCGCTGCC
AATAATGAAACCAGCAAGTCGGTCACTTTCCCAAAGTGTGAAGGT
CTGGATGCTGCCGGAATTGCCGCGAGCGTAAAACGTGATTACCAA
CAAAATCGCGTGGCGCGCTGGGCTGATGATCAAAAAATTGTCGGT
CAGGCCGATCCCGTGGCATGGGTCAGTTTGCAGGACATTCAGGGG
AAAGATGATAAATGGTCAGTACCGCTAACCGTGCGTGGTAAAAGT
GCCGATATTCATTACCAGGTCAGCGTGGACTGCAAAGCGGGAATG
GCGGAATATCAGCGGCGTAGCCCGGGCCAAGGCACGCAGAGCGAA
AACAGCTGCACCCATTTTCCGGGCAACCTGCCGAACATGCTGCGC
GATCTGCGCGATGCGTTTAGCCGCGTGAAAACCTTTTTTCAGATG
AAAGATCAGCTGGATAACCTGCTGCTGAAAGAAAGCCTGCTGGAA
GATTTTAAAGGCTATCTGGGCTGCCAAGCGCTGAGCGAAATGATT
CAGTTTTATCTGGAAGAAGTGATGCCGCAAGCGGAAAACCAAGAT
CCGGATATTAAAGCGCATGTGAACAGCCTGGGCGAAAACCTGAAA
ACCCTGCGCCTGCGCCTGCGCCGCTGCCATCGCTTTCTGCCGTGC
GAAAACAAAAGCAAAGCGGTGGAACAAGTGAAAAACGCGTTTAAC
AAACTGCAAGAAAAGGGAATATATAAGGCAATGTCAGAATTCGAC
ATCTTTATTAACTATATTGAAGCGTATATGACCATGAAAATTCGC
AACTAACAGATAAAAAAAATCCTTAGCTTTCGCTAAGGATGATTA
ATAATCTAGACCAGGCATCAAATAAAACGAAAGGCTCAGTCGAAA
GACTGGGCCTTTCGTTTTATCTGTTGTTTGTCGGTGAACGCTCTC
TACTAGAGTCACACTGGCTCACCTTCGGGTGGGCCTTTCTGCGTT
TATACTCGAGGTGAAGACGAAAGGGCCTCGTGATACGCCTATTTT
TATAGGTTAATGTCATGATAATAATGGTTTCTTAGACGAGCGTTT
TGTTATCAATAAAAAAGGCCCCCCGATTTGGGAGGCCTTTTTAGT
TAGAAAGCTTTAGCGAGGTTTCTTTTTCACCTGAATAGAGTGGTC
ATGATGGCACTGTTCAGGATGACGACACGCTTCTACTTCTACACA
TGCTGAACATAATCCATGTGCTTCAATAACATTATGCCGCAGGGC
AAAACCCATTTTTGCCGCCAGCGTATGCATGATGTCTTCCACGCC
TTCAGCACATTCTTCTTTCACCGCGCCGCAGCGATCGCAAATAAA
CATGGCTGAAGTATGGGTGGGCTGATCGAACAGATGACAGAGCAC
ATAACTGTTGGTGGATTCCACCTTATGCACAAAACCTTGTTCAAG
CAGAAACTCCAGCGCGCGATAAACCGTTGGCGGCTTGGCTTGCGG
TTCAGCTTCGCGCAGCAAATCAAGCAGATCATAAGCGCTGATAGC
GCCATCCTGCAGACTCATCAGGCGCAACACTTCGAGGCGCTGTGG
GGTCAGGCGCACATTACGCTGTGCGCAGATTTTTTCAGCCTGCGC
TAATAACTCCTGCGTTGTGGTCTTTTCCATATATTATCTCCTGAA
TTCGCTAGCACAGTCCCTAGGACTGAGCTAGCTGTAAATCGATAT
CTGGCGAAAATGAGACGTTGATCGGCACGTAAGAGGTTCCAACTT
TCACCATAATGAAATAAGATCACTACCGGGCGTATTTTTTGAGTT
ATCGAGATTTTCAGGAGCTAAGGAAGCTAAAATGGAGAAAAAAAT
CACTGGATATACCACCGTTGATATATCCCAATGGCATCGTAAAGA
ACATTTTGAGGCATTTCAGTCAGTTGCTCAATGTACCTATAACCA
GACCGTTCAGCTGGATATTACGGCCTTTTTAAAGACCGTAAAGAA
AAATAAGCACAAGTTTTATCCGGCCTTTATTCACATTCTTGCCCG
CCTGATGAATGCTCATCCGGAATTTCGTATGGCAATGAAAGACGG
TGAGCTGGTGATATGGGATAGTGTTCACCCTTGTTACACCGTTTT
CCATGAGCAAACTGAAACGTTTTCATCGCTCTGGAGTGAATACCA
CGACGATTTCCGGCAGTTTCTACACATATATTCGCAAGATGTGGC
GTGTTACGGTGAAAACCTGGCCTATTTCCCTAAAGGGTTTATTGA
GAATATGTTTTTCGTCTCAGCCAATCCCTGGGTGAGTTTCACCAG
TTTTGATTTAAACGTGGCCAATATGGACAACTTCTTCGCCCCCGT
TTTCACCATGGGCAAATATTATACGCAAGGCGACAAGGTGCTGAT
GCCGCTGGCGATTCAGGTTCATCATGCCGTCTGTGATGGCTTCCA
TGTCGGCAGAATGCTTAATGAATTACAACAGTACTGCGATGAGTG
GCAGGGCGGGGCGTAATTTGATATCGAGCTCGCTTGGACTCCTGT
TGATAGATCCAGTAATGACCTCAGAACTCCATCTGGATTTGTTCA
GAACGCTCGGTTGCCGCCGGGCGTTTTTTATTGGTGAGAATCCAA
GCACTAGTCATGACCAAAATCCCTTAACGTGAGTTTTCGTTCCAC
TGAGCGTCAGACCCCGTAGAAAAGATCAAAGGATCTTCTTGAGAT
CCTTTTTTTCTGCGCGTAATCTGCTGCTTGCAAACAAAAAAACCA
CCGCTACCAGCGGTGGTTTGTTTGCCGGATCAAGAGCTACCAACT
CTTTTTCCGAAGGTAACTGGCTTCAGCAGAGCGCAGATACCAAAT
ACTGTCCTTCTAGTGTAGCCGTAGTTAGGCCACCACTTCAAGAAC
TCTGTAGCACCGCCTACATACCTCGCTCTGCTAATCCTGTTACCA
GTGGCTGCTGCCAGTGGCGATAAGTCGTGTCTTACCGGGTTGGAC
TCAAGACGATAGTTACCGGATAAGGCGCAGCGGT

pBSIT2

(SEQ ID NO: 18)
CGGGCTGAACGGGGGGTTCGTGCACACAGCCCAGCTTGGAGCGAA
CGACCTACACCGAACTGAGATACCTACAGCGTGAGCATTGAGAAA
GCGCCACGCTTCCCGAAGGGAGAAAGGCGGACAGGTATCCGGTAA
GCGGCAGGGTCGGAACAGGAGAGCGCACGAGGGAGCTTCCAGGGG
GAAACGCCTGGTATCTTTATAGTCCTGTCGGGTTTCGCCACCTCT
GACTTGAGCGTCGATTTTTGTGATGCTCGTCAGGGGGGCGGAGCC
TATGGAAAAACGCCAGCAACGCGGCCTTTTTACGGTTCCTGGCCT
TTTGCTGGCCTTTTGCTCACATGTTCTTTCCTGCGTTATCCCCTG
ATTCTGTGGATAACCGTATTACCGCCTTTGAGTGAGCTGATACCG
CTCGGTCGACTCTAGAGGATCCACGGGTACCTAACGGCAATAAAC
TGTTCACTTCAGTGATATTTAAAATATGCATCCTCTCCCTTTTTT
GTAAGTAATTATTATATCCGTGGGAGAGGAATACACATTGTCAGG
TAATCAATCATGCTGCAATAAATCATCGGCCAGTAAAGTGGAGAT
AGCCTCCATTCTCGAAAAATCCATACTCTCAGCGAAACCATCATC
AATCACTCATCCAGGCGTTTATGGGAGCGTCGCCAATGGCTGCTA
ACAATGCCAGACTTCCCCGTTGCGGAAATTCCACATCCCACAAAT
AGTCACAGTGATTGGGTGTTGAAATGATCCGGATGAGCATGTATC
TTTACGGTTATGTTATAACATAACAGGTAAAGGCTAGCAAAACAA
TAACTAGGATTCGAGGAGAAAAACATGAAAAAAAGAGGGGCGTTT
TTAGGGCTGTTGTTGGTTTCTGCCTGCGCATCAGTTTTCGCTGCC
AATAATGAAACCAGCAAGTCGGTCACTTTCCCAAAGTGTGAAGGT
CTGGATGCTGCCGGAATTGCCGCGAGCGTAAAACGTGATTACCAA
CAAAATCGCGTGGCGCGCTGGGCTGATGATCAAAAAATTGTCGGT
CAGGCCGATCCCGTGGCATGGGTCAGTTTGCAGGACATTCAGGGG
AAAGATGATAAATGGTCAGTACCGCTAACCGTGCGTGGTAAAAGT
GCCGATATTCATTACCAGGTCAGCGTGGACTGCAAAGCGGGAATG
GCGGAATATCAGCGGCGTAGCCCGGGCCAAGGCACGCAGAGCGAA
AACAGCTGCACCCATTTTCCGGGCAACCTGCCGAACATGCTGCGC
GATCTGCGCGATGCGTTTAGCCGCGTGAAAACCTTTTTTCAGATG
AAAGATCAGCTGGATAACCTGCTGCTGAAAGAAAGCCTGCTGGAA
GATTTTAAAGGCTATCTGGGCTGCCAAGCGCTGAGCGAAATGATT
CAGTTTTATCTGGAAGAAGTGATGCCGCAAGCGGAAAACCAAGAT
CCGGATATTAAAGCGCATGTGAACAGCCTGGGCGAAAACCTGAAA
ACCCTGCGCCTGCGCCTGCGCCGCTGCCATCGCTTTCTGCCGTGC
GAAAACAAAAGCAAAGCGGTGGAACAAGTGAAAAACGCGTTTAAC
AAACTGCAAGAAAAGGGAATATATAAGGCAATGTCAGAATTCGAC
ATCTTTATTAACTATATTGAAGCGTATATGACCATGAAAATTCGC
AACTAAGGATCCATAATCAGGATCAATAAAACTGTTGCAGAAATG
ATTTCATTCATAACTCAAATTCCCTGATAATTGCCGCGGACTTTC
TGCGTGCTAACAAAGCAGGATAAGTCGCATTACTGATGGCTTCGC
TATCATTGATTAATTTCACTTGCGACTTTGGCTGCTTTTTGTATG
GTGAAAGATGTGCCAGGAGGAGACCGGCACATTTATACAGCACAC
ATCTTTGCAGGAAAAAACGCTTATGAAAAATGTTGGTTTTATCGG
CTGGCGCGGTATGGTCGGCTCCGTTCTCATGCAACGCATGGTTGA
AGAGCGCGACTTCGACGCCATTCGCCCTGTCTTCTTTTCTACTTC
TCAGCTTGGTCAGGCTGCGCCGTCTTTTGGCGGAACCACTGGCAC
ACTTCAGGATGCCTTTGATCTGGAGGCGCTAAAGGCCCTCGATAT
CATTGTGACCTGTCAGGGCGGCGATTATACCAACGAAATCTATCC
AAAGCTTCGTGAAAGCGGATGGCAAGGTTACTGGATTGACGCAGC
ATCATCTCTGCGCATGAAAGATGACGCCATCATCATTCTTGACCC
CGTCAATCAGGACGTCATTACCGACGGATTAAATAATGGCATCAG
GACTTTTGTTGGCGGTAACTGTACCGTAAGCCTGATGTTGATGTC
GCTGGGTGGTTTATTCGCCAATGATCTTGTTGATTGGGTGTCCGT
TGCAACCTACCAGGCCGCTTCCGGCGGTGGTGCGCGACATATGCG
TGAGTTATTAACCCAAATGGGCCATCTGTATGGCCATGTGGCAGA
TGAACTCGCGAACCCGTCCTCTGCTATTCTCGATATCGAACGCAA
AGTCACAACCTTAACCCGTAGCGGTGAGCTGCCGGTAGATAACTT
TGGCGTGCCGCTGGCGGGTAGCCTGATTCCGTGGATCGACAAACA
GCTTGATAACGGTCAGAGCCGCGAAGAGTGGAAAGGGCAGGCGGA
AACCAACAAGATCCTCAACACATCTTCCGTAATTCCGGTAGATGG
TTTATGTGTGCGTGTCGGGGCATTGCGCTGCCACAGCCAGGCATT
CACTATTAAATTGAAAAAAGATGTGTCTATTCCGACCGTGGAAGA
ACTGCTGGCTGCGCACAATCCGTGGGCGAAAGTTGTTCCAAACGA
TCGGGAAATCACTATGCGTGAGCTAACCCCAGCTGCCGTTACCGG
CACGCTGACCACGCCGGTAGGCCGTCTGCGTAAGCTGAATATGGG
ACCAGAGTTCCTGTCAGCCTTTACCGTGGGCGACCAGCTGCTGTG
GGGGGCCGCGGAGCCGCTGCGTCGGATGCTTCGTCAACTGGCGTA
ACAGATAAAAAAAATCCTTAGCTTTCGCTAAGGATGATTAATAAT
CTAGACCAGGCATCAAATAAAACGAAAGGCTCAGTCGAAAGACTG
GGCCTTTCGTTTTATCTGTTGTTTGTCGGTGAACGCTCTCTACTA
GAGTCACACTGGCTCACCTTCGGGTGGGCCTTTCTGCGTTTATAC
TCGAGGTGAAGACGAAAGGGCCTCGTGATACGCCTATTTTTATAG
GTTAATGTCATGATAATAATGGTTTCTTAGACGAGCGTTTTGTTA
TCAATAAAAAAGGCCCCCCGATTTGGGAGGCCTTTTTAGTTAGAA
AGCTTTAGCGAGGTTTCTTTTTCACCTGAATAGAGTGGTCATGAT
GGCACTGTTCAGGATGACGACACGCTTCTACTTCTACACATGCTG
AACATAATCCATGTGCTTCAATAACATTATGCCGCAGGGCAAAAC
CCATTTTTGCCGCCAGCGTATGCATGATGTCTTCCACGCCTTCAG
CACATTCTTCTTTCACCGCGCCGCAGCGATCGCAAATAAACATGG
CTGAAGTATGGGTGGGCTGATCGAACAGATGACAGAGCACATAAC
TGTTGGTGGATTCCACCTTATGCACAAAACCTTGTTCAAGCAGAA
ACTCCAGCGCGCGATAAACCGTTGGCGGCTTGGCTTGCGGTTCAG
CTTCGCGCAGCAAATCAAGCAGATCATAAGCGCTGATAGCGCCAT
CCTGCAGACTCATCAGGCGCAACACTTCGAGGCGCTGTGGGGTCA
GGCGCACATTACGCTGTGCGCAGATTTTTTCAGCCTGCGCTAATA
ACTCCTGCGTTGTGGTCTTTTCCATATATTATCTCCTGAATTCGC
TAGCACAGTCCCTAGGACTGAGCTAGCTGTAAATCGATATCTGGC
GAAAATGAGACGTTGATCGGCACGTAAGAGGTTCCAACTTTCACC
ATAATGAAATAAGATCACTACCGGGCGTATTTTTTGAGTTATCGA
GATTTTCAGGAGCTAAGGAAGCTAAAATGGAGAAAAAAATCACTG
GATATACCACCGTTGATATATCCCAATGGCATCGTAAAGAACATT
TTGAGGCATTTCAGTCAGTTGCTCAATGTACCTATAACCAGACCG
TTCAGCTGGATATTACGGCCTTTTTAAAGACCGTAAAGAAAAATA
AGCACAAGTTTTATCCGGCCTTTATTCACATTCTTGCCCGCCTGA
TGAATGCTCATCCGGAATTTCGTATGGCAATGAAAGACGGTGAGC
TGGTGATATGGGATAGTGTTCACCCTTGTTACACCGTTTTCCATG
AGCAAACTGAAACGTTTTCATCGCTCTGGAGTGAATACCACGACG
ATTTCCGGCAGTTTCTACACATATATTCGCAAGATGTGGCGTGTT
ACGGTGAAAACCTGGCCTATTTCCCTAAAGGGTTTATTGAGAATA
TGTTTTTCGTCTCAGCCAATCCCTGGGTGAGTTTCACCAGTTTTG
ATTTAAACGTGGCCAATATGGACAACTTCTTCGCCCCCGTTTTCA
CCATGGGCAAATATTATACGCAAGGCGACAAGGTGCTGATGCCGC
TGGCGATTCAGGTTCATCATGCCGTCTGTGATGGCTTCCATGTCG
GCAGAATGCTTAATGAATTACAACAGTACTGCGATGAGTGGCAGG
GCGGGGCGTAATTTGATATCGAGCTCGCTTGGACTCCTGTTGATA
GATCCAGTAATGACCTCAGAACTCCATCTGGATTTGTTCAGAACG
CTCGGTTGCCGCCGGGCGTTTTTTATTGGTGAGAATCCAAGCACT
AGTCATGACCAAAATCCCTTAACGTGAGTTTTCGTTCCACTGAGC
GTCAGACCCCGTAGAAAAGATCAAAGGATCTTCTTGAGATCCTTT
TTTTCTGCGCGTAATCTGCTGCTTGCAAACAAAAAAACCACCGCT
ACCAGCGGTGGTTTGTTTGCCGGATCAAGAGCTACCAACTCTTTT
TCCGAAGGTAACTGGCTTCAGCAGAGCGCAGATACCAAATACTGT
CCTTCTAGTGTAGCCGTAGTTAGGCCACCACTTCAAGAACTCTGT
AGCACCGCCTACATACCTCGCTCTGCTAATCCTGTTACCAGTGGC
TGCTGCCAGTGGCGATAAGTCGTGTCTTACCGGGTTGGACTCAAG
ACGATAGTTACCGGATAAGGCGCAGCGGT

pBSITM

(SEQ ID NO: 19)
TGGAAGATGCCAGGAAGATACTTAACAGGGAAGTGAGAGGGCCGC
GGCAAAGCCGTTTTTCCATAGGCTCCGCCCCCCTGACAAGCATCA
CGAAATCTGACGCTCAAATCAGTGGTGGCGAAACCCGACAGGACT
ATAAAGATACCAGGCGTTTCCCCCTGGCGGCTCCCTCGTGCGCTC
TCCTGTTCCTGCCTTTCGGTTTACCGGTGTCATTCCGCTGTTATG
GCCGCGTTTGTCTCATTCCACGCCTGACACTCAGTTCCGGGTAGG
CAGTTCGCTCCAAGCTGGACTGTATGCACGAACCCCCCGTTCAGT
CCGACCGCTGCGCCTTATCCGGTAACTATCGTCTTGAGTCCAACC
CGGAAAGACATGCAAAAGCACCACTGGCAGCAGCCACTGGTAATT
GATTTAGAGGAGTTAGTCTTGAAGTCATGCGCCGGTTAAGGCTAA
ACTGAAAGGACAAGTTTTGGTGACTGCGCTCCTCCAAGCCAGTTA
CCTCGGTTCAAAGAGTTGGTAGCTCAGAGAACCTTCGAAAAACCG
CCCTGCAAGGCGGTTTTTTCGTTTTCAGAGCAAGAGATTACGCGC
AGACCAAAACGATCTCAAGAAGATCATCTTATTAAGGGGTCTGAC
GCTCAGTGGAACGAAAAGTATATATGAGTAAACTTGGTCTGACAG
CTCGAGCTTACCCGTCTTACTGTCCCTAGTGCTTGGATTCTCACC
AATAAAAAACGCCCGGCGGCAACCGAGCGTTCTGAACAAATCCAG
ATGGAGTTCTGAGGTCATTACTGGATCTATCAACAGGAGTCCAAG
CGAGCTCGTAAACTTGGTCTGACAGTTACCAATGCTTAATCAGTG
AGGCACCTATCTCAGCGATCTGTCTATTTCGTTCATCCATAGTTG
CCTGACTCCCCGTCGTGTAGATAACTACGATACGGGAGGGCTTAC
CATCTGGCCCCAGTGCTGCAATGATACCGCGTGATCCACGCTCAC
CGGCTCCAGATTTATCAGCAATAAACCAGCCAGCCGGAAGGGCCG
AGCGCAGAAGTGGTCCTGCAACTTTATCCGCCTCCATCCAGTCTA
TTAATTGTTGCCGGGAAGCTAGAGTAAGTAGTTCGCCAGTTAATA
GTTTGCGCAACGTTGTTGCCATTGCTACAGGCATCGTGGTGTCAC
GCTCGTCGTTTGGTATGGCTTCATTCAGCTCCGGTTCCCAACGAT
CAAGGCGAGTTACATGATCCCCCATGTTGTGCAAAAAATCGGTTA
GCTCCTTCGGTCCTCCGATCGTTGTCAGAAGTAAGTTGGCCGCAG
TGTTATCACTCATGGTTATGGCAGCACTGCATAATTCTCTTACTG
TCATGCCATCCGTAAGATGCTTTTCTGTGACTGGTGAGTACTCAA
CCAAGTCATTCTGAGAATAGTGTATGCGGCGACCGAGTTGCTCTT
GCCCGGCGTCAATACGGGATAATACCGCGCCACATAGCAGAACTT
TAAAAGTGCTCATCATTGGAAAACGTTCTTCGGGGCGAAAACTCT
CAAGGATCTTACCGCTGTTGAGATCCAGTTCGATGTAACCCACTC
GTGCACCCAACTGATCTTCAGCATCTTTTACTTTCACCAGCGTTT
CTGGGTGAGCAAAAACAGGAAGGCAAAATGCCGCAAAAAAGGGAA
TAAGGGCGACACGGAAATGTTGAATACTCATACTCTTCCTTTTTC
AATATTATTGAAGCATTTATCAGGGTTATTGTCTCATGAGCGGAT
ACATATTTGAATGTTACGTGCCCGATCAACTGCGTTTATATACTA
GTAGCGGCAGCTGCAGTCCGGCAAAAAAGGGCAAGGTGTCACCAC
CCTGCCCTTTTTCTTTAAAACCGAAAAGATTACTTCGCGTTATGC
AGGCTTCCTCGCTCACTGACTCGCTGCGCTCGGTCGTTCGGCTGC
GGCGAGCGGTATCAGCTCACTCAAAGGCGGTAATTTGACAGCTAG
CTCAGTCCTAGGTATAATGCTAGCAGTTCGATGAGAGCGATAACC
AATCATCAGATAACTATGGCGGCACGTGCATTAACCACGGTTGTA
TCCCGTCTAAAGTACTCGTCTTTAGTTGCGAATTTTCATGGTCAT
ATACGCTTCAATATAGTTAATAAAGATGTCGAATTCTGACATTGC
CTTATATATTCCCTTTTCTTGCAGTTTGTTAAACGCGTTTTTCAC
TTGTTCCACCGCTTTGCTTTTGTTTTCGCACGGCAGAAAGCGATG
GCAGCGGCGCAGGCGCAGGCGCAGGGTTTTCAGGTTTTCGCCCAG
GCTGTTCACATGCGCTTTAATATCCGGATCTTGGTTTTCCGCTTG
CGGCATCACTTCTTCCAGATAAAACTGAATCATTTCGCTCAGCGC
TTGGCAGCCCAGATAGCCTTTAAAATCTTCCAGCAGGCTTTCTTT
CAGCAGCAGGTTATCCAGCTGATCTTTCATCTGAAAAAAGGTTTT
CACGCGGCTAAACGCATCGCGCAGATCGCGCAGCATGTTCGGCAG
GTTGCCCGGAAAATGGGTGCAGCTGTTTTCGCTCTGCGTGCCTTG
GCCCGGGCTACGCCGCTGATATTCCGCCATTCCCGCTTTGCAGTC
CACGCTGACCTGGTAATGAATATCGGCACTTTTACCACGCACGGT
TAGCGGTACTGACCATTTATCATCTTTCCCCTGAATGTCCTGCAA
ACTGACCCATGCCACGGGATCGGCCTGACCGACAATTTTTTGATC
ATCAGCCCAGCGCGCCACGCGATTTTGTTGGTAATCACGTTTTAC
GCTCGCGGCAATTCCGGCAGCATCCAGACCTTCACACTTTGGGAA
AGTGACCGACTTGCTGGTTTCATTATTGGCAGCGAAAACTGATGC
GCAGGCAGAAACCAACAACAGCCCTAAAAACGCCCCTCTTTTTTT
CATGTTTTTCTCCTCGAATCCTAGTTATTGTTTTCTATTTTATTA
TGGAAGTTTGTTCACTCAACATTGCAAGACTGTACATACTTCCAT
AGTTTATTAATATAAACGCAGAAAGGCCCACCCGAAGGTGAGCCA
GTGTGACTCTAGTAGAGAGCGTTCACCGACAAACAACAGATAAAA
CGAAAGGCCCAGTCTTTCGACTGAGCCTTTCGTTTTATTTGATGC
CTGGCTAGGGCCTACGCCCGGTAGTGATCTTATTTCATTATGGTG
AAAGTTGGAACCTCTTACGGGTAGAGACCCACGAGGCAGACCTCA
GCGCTAGCGGAGTGTATACTGGCTTACTATGTTGGCACTGATGAG
GGTGTCAGTGAAGTGCTTCATGTGGCAGGAGAAAAAAGGCTGCAC
CGGTGCGTCAGCAGAATATGTGATACAGGATATATTCCGCTTCCT
CGCTCATTACGCCAGTTGACGAAGCATCCGACGCAGCGGCTCCGC
GGCCCCCCACAGCAGCTGGTCGCCCACGGTAAAGGCTGACAGGAA
CTCTGGTCCCATATTCAGCTTACGCAGACGGCCTACCGGCGTGGT
CAGCGTGCCGGTAACGGCAGCTGGGGTTAGCTCACGCATAGTGAT
TTCCCGATCGTTTGGAACAACTTTCGCCCACGGATTGTGCGCAGC
CAGCAGTTCTTCCACGGTCGGAATAGACACATCTTTTTTCAATTT
AATAGTGAATGCCTGGCTGTGGCAGCGCAATGCCCCGACACGCAC
ACATAAACCATCTACCGGAATTACGGAAGATGTGTTGAGGATCTT
GTTGGTTTCCGCCTGCCCTTTCCACTCTTCGCGGCTCTGACCGTT
ATCAAGCTGTTTGTCGATCCACGGAATCAGGCTACCCGCCAGCGG
CACGCCAAAGTTATCTACCGGCAGCTCACCGCTACGGGTTAAGGT
TGTGACTTTGCGTTCGATATCGAGAATAGCAGAGGACGGGTTCGC
GAGTTCATCTGCCACATGGCCATACAGATGGCCCATTTGGGTTAA
TAACTCACGCATATGTCGCGCACCACCGCCGGAAGCGGCCTGGTA
GGTTGCAACGGACACCCAATCAACAAGATCATTGGCGAATAAACC
ACCCAGCGACATCAACATCAGGCTTACGGTACAGTTACCGCCAAC
AAAAGTCCTGATGCCATTATTTAATCCGTCGGTAATGACGTCCTG
ATTGACGGGGTCAAGAATGATGATGGCGTCATCTTTCATGCGCAG
AGATGATGCTGCGTCAATCCAGTAACCTTGCCATCCGCTTTCACG
AAGCTTTGGATAGATTTCGTTGGTATAATCGCCGCCCTGACAGGT
CACAATGATATCGAGGGCCTTTAGCGCCTCCAGATCAAAGGCATC
CTGAAGTGTGCCAGTGGTTCCGCCAAAAGACGGCGCAGCCTGACC
AAGCTGAGAAGTAGAAAAGAAGACAGGGCGAATGGCGTCGAAGTC
GCGCTCTTCAACCATGCGTTGCATGAGAACGGAGCCGACCATACC
GCGCCAGCCGATAAAACCAACATTTTTCATAAGCGTTTTTTCCTG
CAAAGATGTGTGCTGTATAAATGTGCCGGTCTCCTCCTGGCACAT
CTTTCACCATACAAAAAGCAGCCAAAGTCGCAAGTGAAATTAATC
AATGATAGCGAAGCCATCAGTAATGCGACTTATCCTGCTTTGTTA
GCACGCAGAAAGTCCGCGGCAATTATCAGGGAATTTGAGTTATGA
ATGAAATCATTTCTGCAACAGTTTTATTGATCCTGATTATGGATC
C

In certain embodiments, the system and/or composition comprises all or a portion of a polynucleotide from any of SEQ ID NO:9-19. The portion may comprise one or more of a promoter, gene of interest (which may be Zur), integrase, recombinase, target site, reporter gene, therapeutic gene, or a combination thereof.

### III. KITS OF THE DISCLOSURE

Any of the compositions described herein may be comprised in a kit. In particular embodiments, bacteria, one or more polynucleotides, including vectors of any kind, one or more primers to produce certain desired sequences, one or more expression constructs, and/or one or more reagents to manipulate any of these may be encompassed in a kit. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the compositions and any other reagent containers in close confinement for commercial sale. Such containers may include injection or blow-molded plastic containers into which the desired vials are retained. The bacteria may be cryopreserved, in some instances.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Identification and Use of Calprotectin-Responsive Promoters

The present example concerns identification and use of promoters that are responsive to calprotectin. In particular embodiments, the use concerns monitoring inflammation in the gut with a biosensor that utilizes calprotectin-responsive promoters to regulate expression of a detectable gene product and/or one or more therapeutic genes.

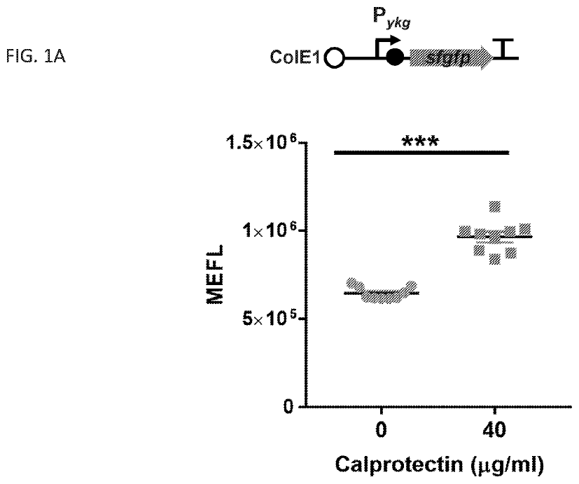

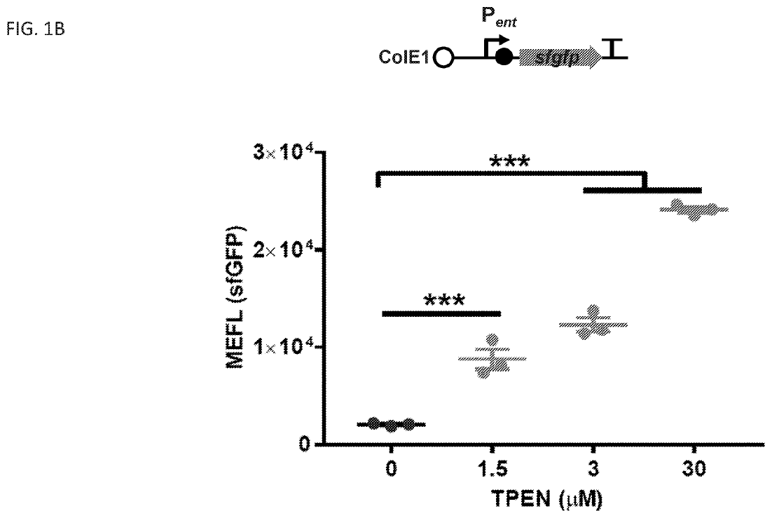

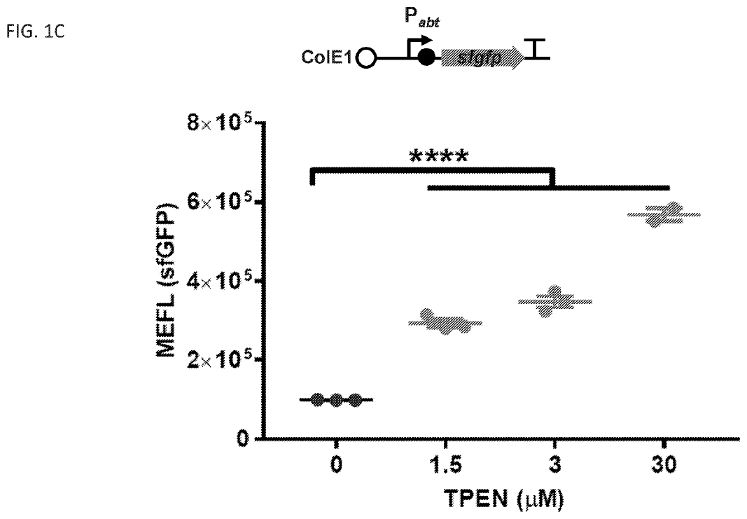

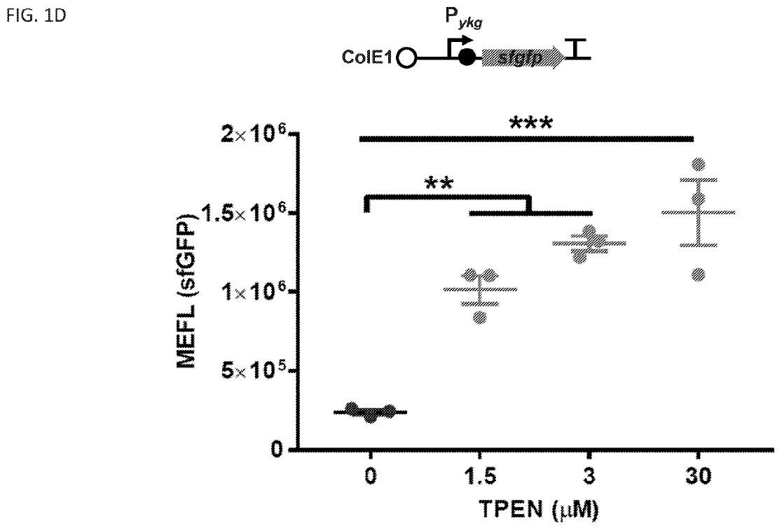

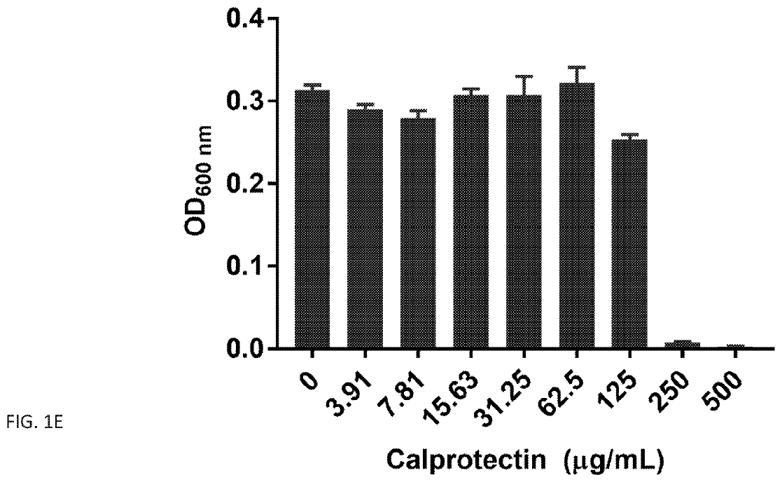

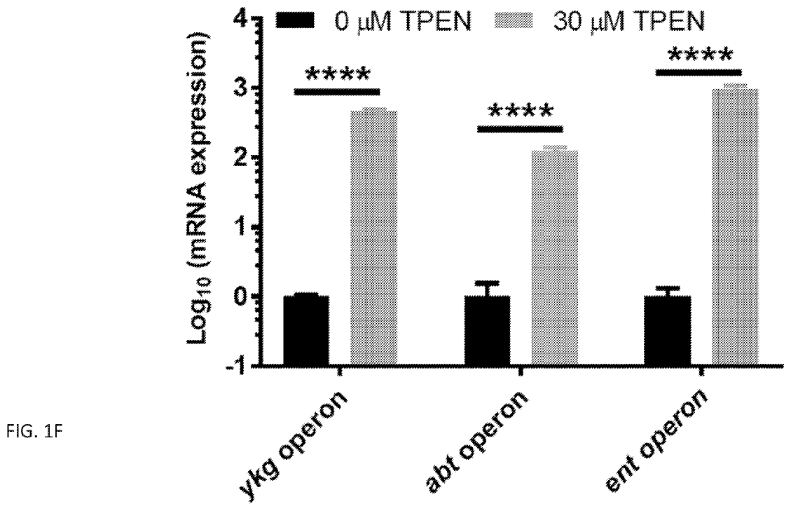

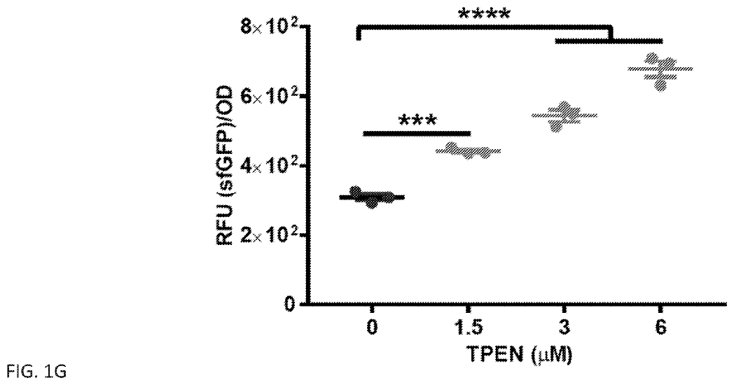

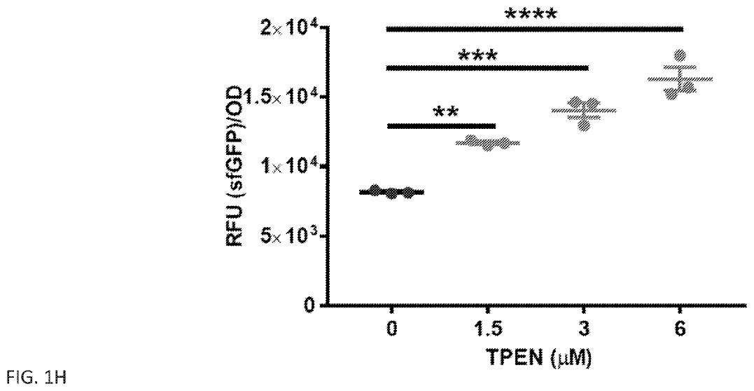

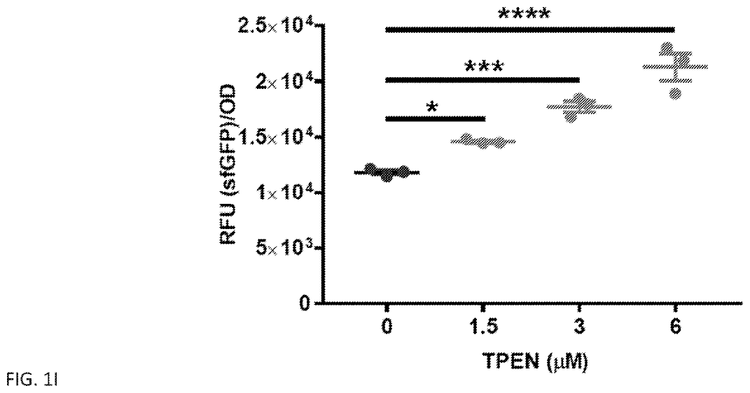

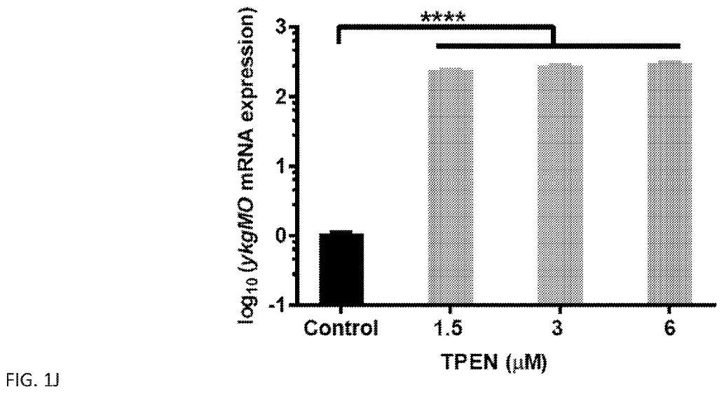

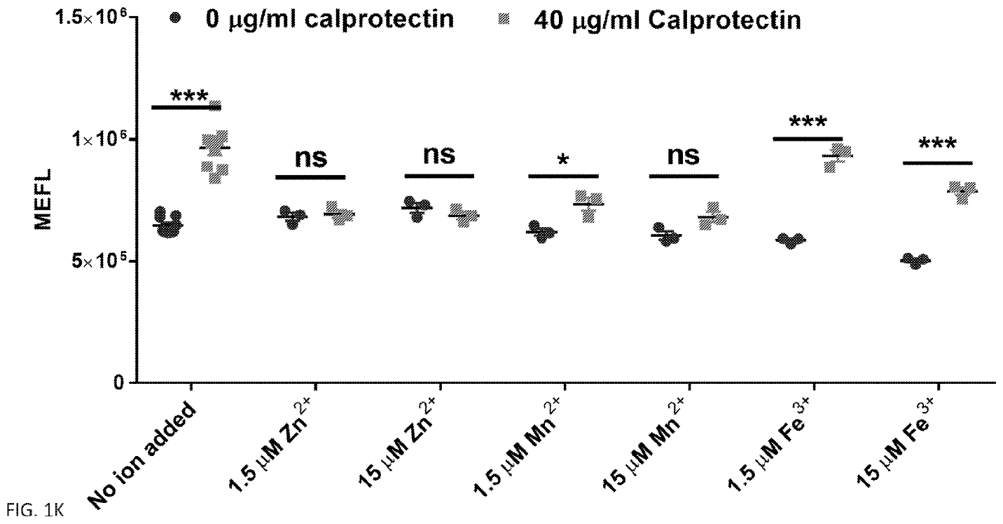

Identification of E. coli Nissle promoters responsive to calprotectin. Calprotectin is a neutrophil associated protein that exhibits nutritional immunity against microbes by binding to and sequestering metals required for growth. Currently, fecal calprotectin levels are used as a non-invasive way of monitoring intestinal inflammation, however compliance with such tests is low due to the need for patients to handle their own feces. Therefore, the development of an ingestible bacterial biosensor that would sense and report intestinal inflammation was contemplated. To this end, promoters were identified in Escherichia coli Nissle 1917 (EcN) that are upregulated during exposure to calprotectin. To do this the minimal inhibitory concentration of calprotectin incubated with EcN in vitro was first identified. Two-fold dilutions of calprotectin were incubated with 10⁴ EcN cells overnight and the lowest dilution (256 μg/mL) that inhibited growth was identified (FIG. 1E).

It was considered that exposing EcN to a sub-inhibitory concentration of calprotectin that was near the MIC would yield the best opportunity to identify physiologically relevant promoters that respond to calprotectin. The minimal inhibitory concentration of calprotectin (250 μg/mL) was first identified using EcN growth in LB media (FIG. 1E). Therefore, EcN were grown into the mid-exponential phase in LB media and then treated cultures with 125 g/mL of calprotectin for 30 minutes or without calprotectin as a negative control. As an additional control, a mutant version was used of calprotectin that lacks the ability to bind metals, rendering the protein unable to carry out nutritional immunity. A pilot RNA sequencing experiment was performed on the samples and genes were identified that were significantly upregulated in calprotectin-treated bacteria compared to untreated controls. The three most highly expressed promoters induced by calprotectin were the alternative ribosomal protein operon (ykgMO, upregulated up to 10.3 fold), the siderophore enterobactin operon (ent, upregulated up to 4.8 fold), and an ABC transporter operon of unknown function (abt). Each of these genes were subsequently verified as being upregulated by incubating EcN with 30 μM of the metal chelator TPEN [N,N,N′, N′-tetrakis(2-pyridinylmethyl)-1,2-ethanediamine] in LB media for 30 minutes by RT-qPCR (Table 1, FIG. 1F).

TABLE 1
Calprotectin-Induced Genes Chosen for Sensor Construction

| | | Fold-Change |
|---|---|---|
| | | in Gene |
| | Host Microbe | Expression |
| [symbol] [symbol] operon | Escherichia coli [symbol] | 4.38-4.78 |
| 50S Ribosomal Protein L36/L31 type B | Escherichia coli [symbol] | 4.20-10.26 |
| ATP-binding cassette transporter cluster | Escherichia coli [symbol] | 3.80-9.44 |

Data are ranges of expression is calprotectin-induced gene clusters over untreated cells

Selection of the ykgMO promoter to sense calprotectin levels. To assess the ykg, ent, and abt promoters as potential sensors of the metal sequestration activity of calprotectin, P_{ykg}, Pₑₙₜ, and Paht were cloned into plasmid pColE1 driving the expression of superfolder GFP (sfgfp). Sensor fluorescence was next analyzed in M9 minimal medium and used metal chelator TPEN to mimic the metal depletion of calprotectin. The data showed that these three promoters constructs were all activated between 2-3 fold by TPEN compared to the control, confirming what was observed in the RNAseq and RT-qPCR analysis (FIGS. 1B-1D and FIGS. 1G-1I). Among them, the ykg promoter responded to TPEN at as low as 1.5 M in M9 media and showed higher fluorescence compared to the ent and abt promoters (FIG. 1D and FIG. 1J).

A focus was chosen on the ykgMO promoter as an in vivo biosensor, denoted as BSI0 (EcN harboring plasmid bacterial sensor of inflammation). First, this operon was highly expressed when induced as it encodes ribosomal proteins required for ribosome function during zinc starvation28. This allowed for the engineering of a sensor with a large dynamic range of expression. Second, the ykg operon is regulated by the zinc sensing transcriptional repressor Zur, which requires the depletion of zinc to extremely low levels to become activated²⁸. Therefore, one would expect this operon will only be activated under extreme zinc depletion conditions, such as at sites of neutrophil infiltration and calprotectin release during an acute inflammatory response. Sequestration of metals such as zinc by calprotectin during neutrophil infiltration during inflammation has been directly demonstrated in vivo^{29.30}.

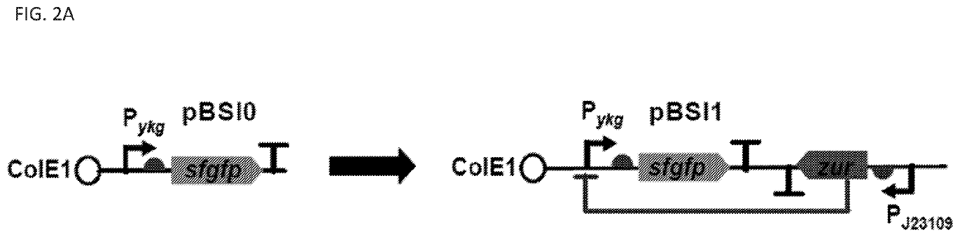

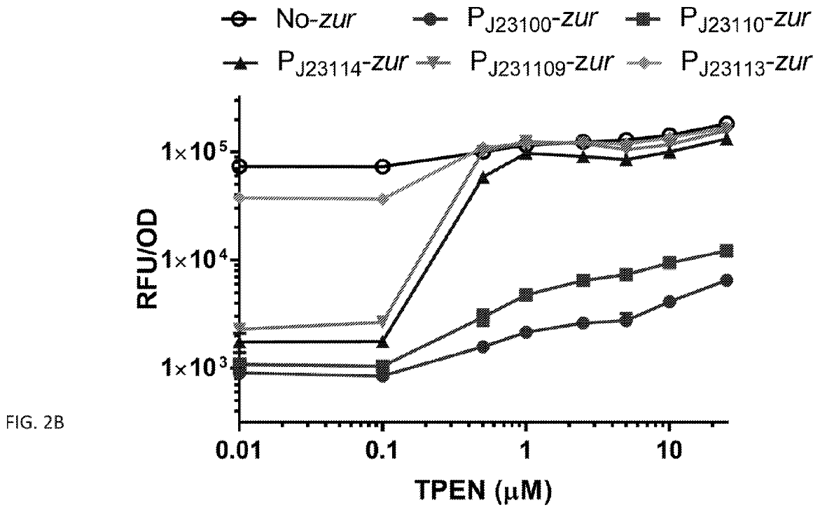

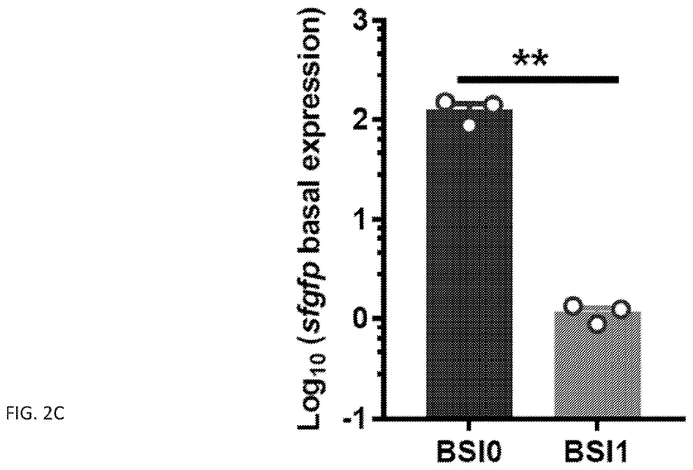

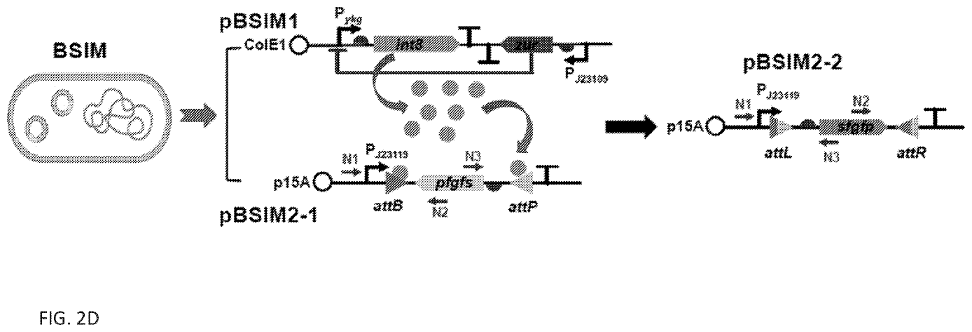

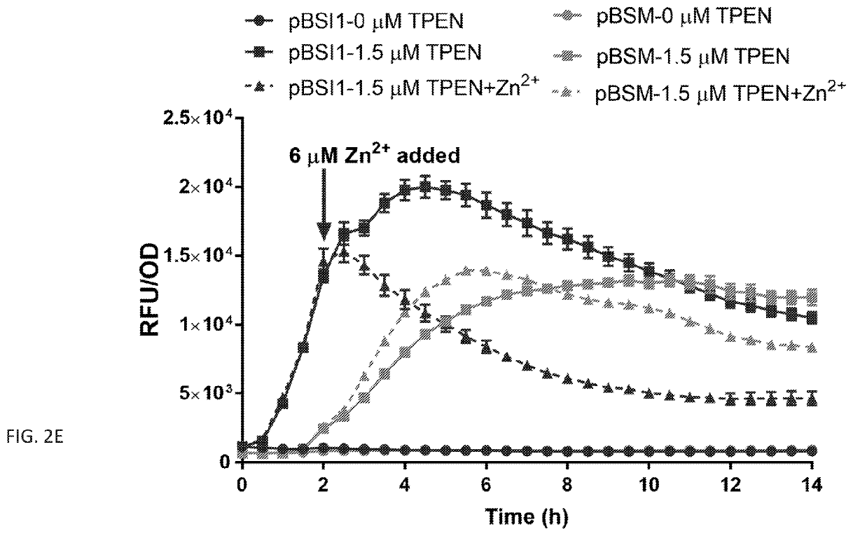

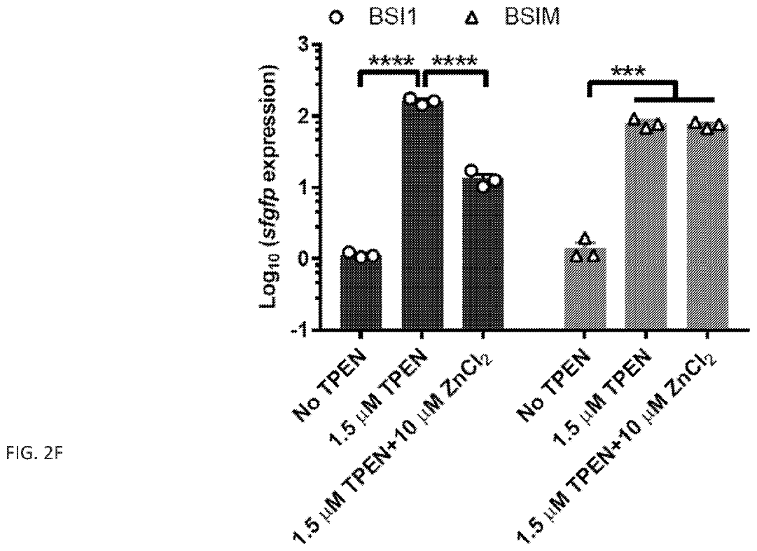

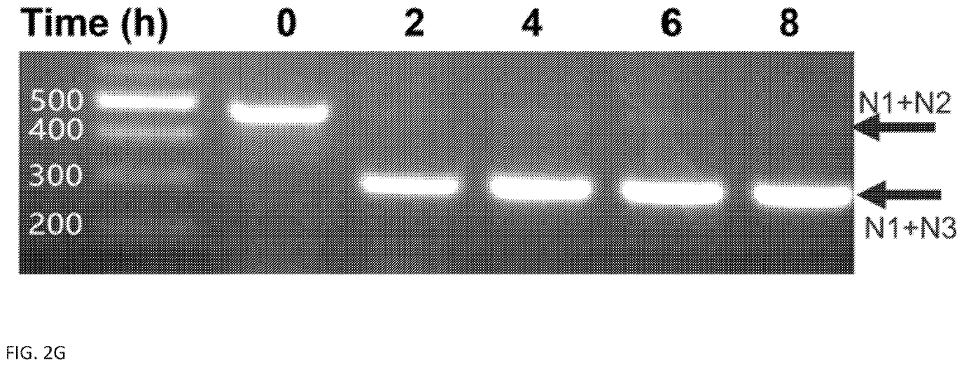

To validate that the P_{ykg} is sensitive to zinc but not other metals bound by calprotectin, BSI0 was incubated with 40 μg/mL of calprotectin with or without supplementation of an equimolar amount of zinc, manganese, or iron. Incubation with Zn, but not Mn or Fe, was able to totally repress the activation of BSI0 in the presence of calprotectin (FIG. 2D), confirming that zinc is the critical metal for regulating this promoter.

Optimization of the ykgMO promoter for sensing inflammation during nutritional immunity.

To increase the dynamic range of the P_{ykg} expression, the high level of basal expression when the operon is not activated by zinc limitation needed to be reduced. It was considered that the basal level of expression was high because the ykg promoter was being expressed from a multicopy plasmid while the Zur repressor was being synthesized from the chromosome, altering their normal stoichiometry. To identify the optimal level of Zur to reduce the basal expression while maintaining the ability to respond to zinc limitation, several constructs were tested in which the zur gene was driven by constitutive promoters of increasing strength on the same plasmid as the ykg promoter (FIG. 2A) . . . . J23114 and J23109 were able to significantly reduce the basal expression from the ykg promoter by 100-fold while retaining the ability to be maximally induced by zinc depletion with the metal chelating agent TPEN (FIG. 2B). Other promoters were either too weak (J23113) to reduce the basal level of promoter expression or were too strong (J23100, J23110) to allow induction of the P_{ykg} upon zinc depletion. Additional studies utilized the J23109 promoter construct, now denoted as pBSI1 (bacterial sensor of inflammation). Basal transcription of BSI1 was significantly lower compared to BSI0 (FIG. 2C).

Establishment of memory to record sensing of inflammation. Because it was considered that zinc sequestration to levels that activate the sensor will only occur at sites of active inflammation and neutrophil infiltration, it was contemplated that a useful biosensor would need to become permanently activated upon sensing inflammation, such as for future reporting in feces. A two-plasmid system was developed in which the activation of the ykg promoter by zinc depletion drives the expression of integrase 8 in the first plasmid, pBSIM1 (FIG. 2D). In the second plasmid, the sfgfp gene is flanked by attachment sites (attB, attP) that are recognized by integrase 8. The expression and activity of integrase 8 will flip the orientation of the sfgfp gene such that the gene is now in under the control of a strong constitutive promoter (J23119) in pBSIM2 (FIG. 2D and FIG. 2G). The resultant strain was named BSIM (bacterial sensor of inflammation with memory) to denote the addition of the memory circuit to the sensor (FIG. 2D).

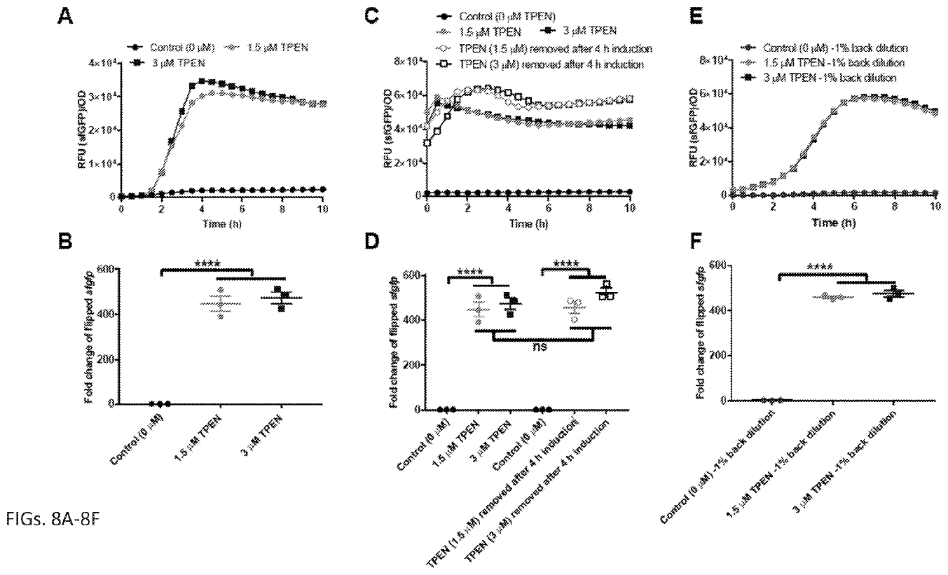

To test if BSIM could be activated by zinc depletion and remain on after restoration of zinc concentration, BSIM cells were cultured in M9 media and induced with 1.5 μM of TPEN at mid-log phase. After 2 hours induction zinc (10 μM) was added to the cultures. As a control, the BSI1 biosensor that lacks the memory switch and should be turned off by the addition of zinc was tested. As expected, both constructs were strongly activated by TPEN, with the memory construct showing slightly slower induction, most likely due to the need to express the integrase and flip the orientation of the sfgfp gene (FIG. 2E). After zinc addition, the sfgfp of BSIM remained strongly expressed as expected, while the sfgfp expression of BSI1 declined rapidly (FIGS. 2E-F). The stable expression of sfgfp in BSIM was also verified by dilution of TPEN treated cultures into fresh medium without TPEN (FIGS. 8A-8F). The data demonstrated the integration of the memory circuit allows for continued expression of a reporter upon sensing of metal depletion after metal concentrations have been restored.

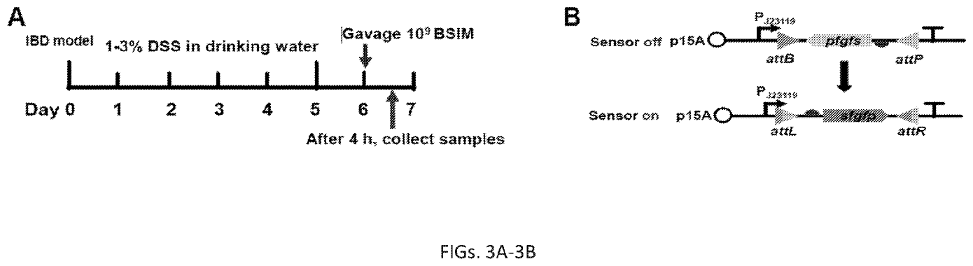

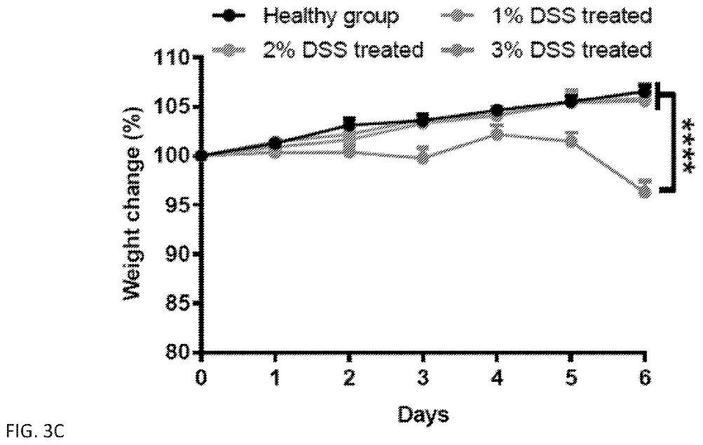

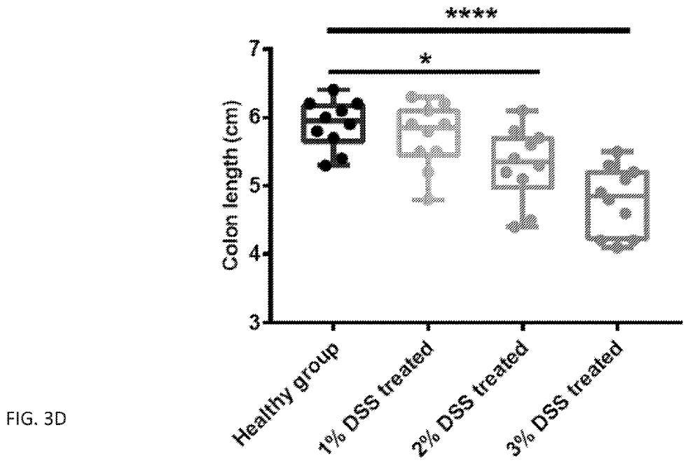

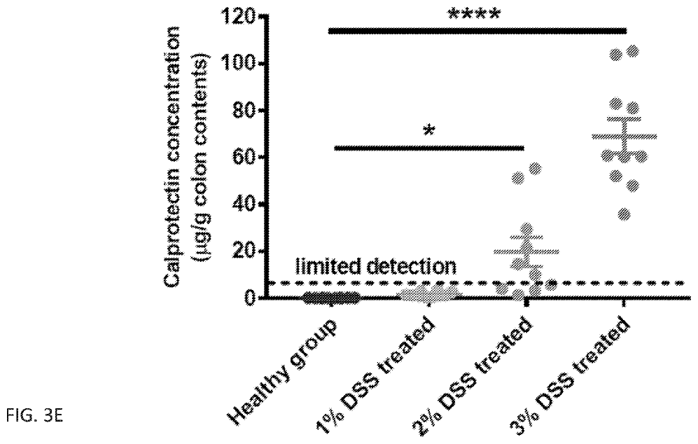

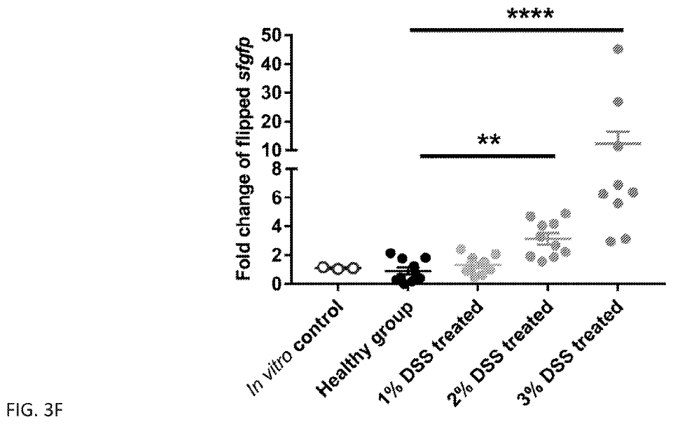

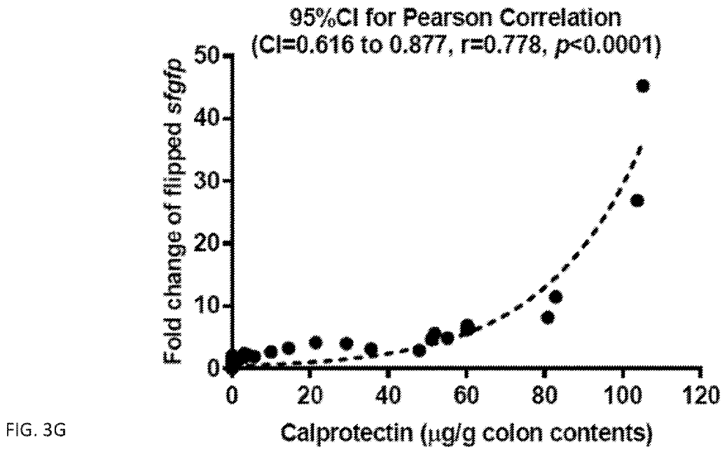

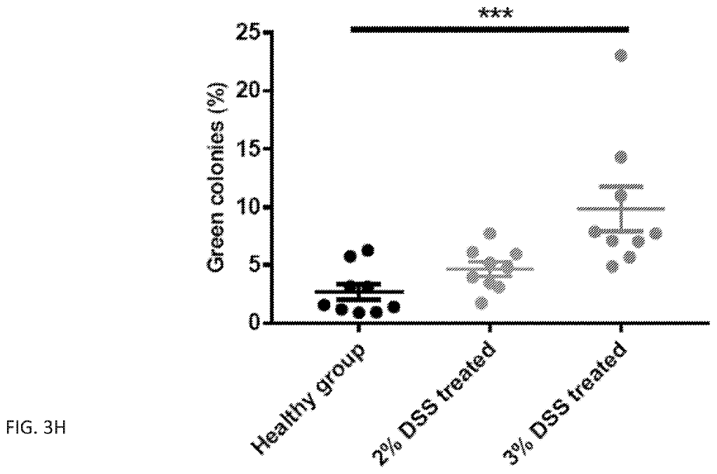

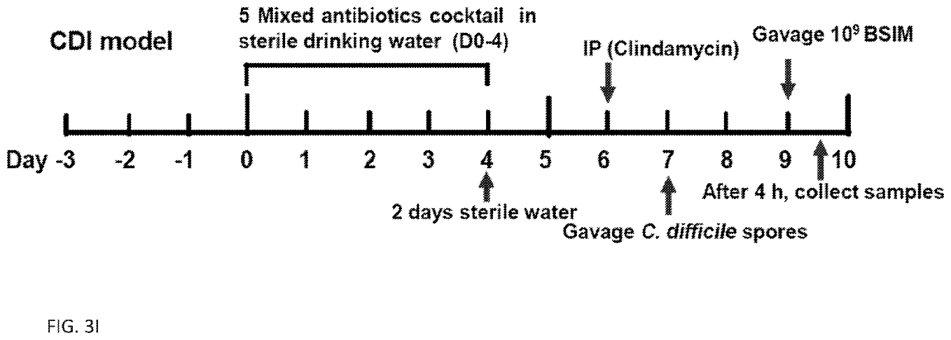

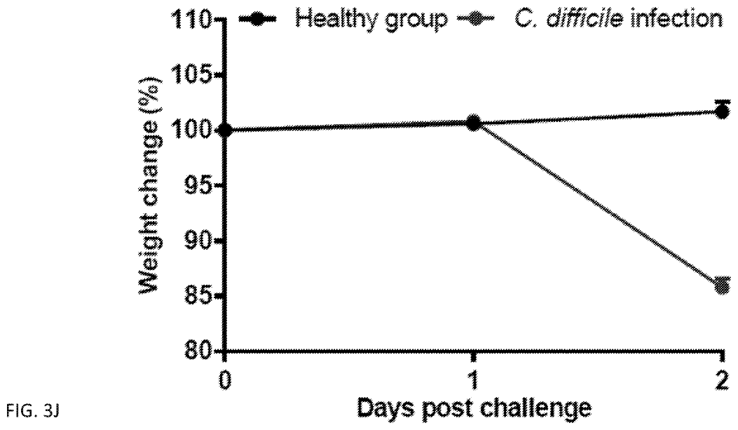

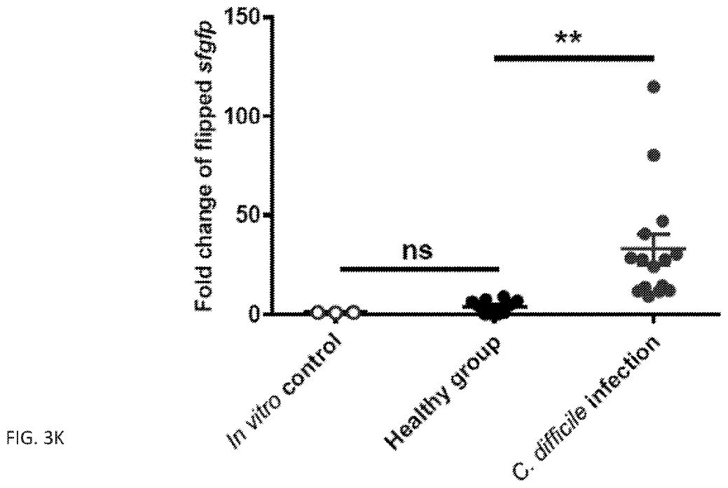

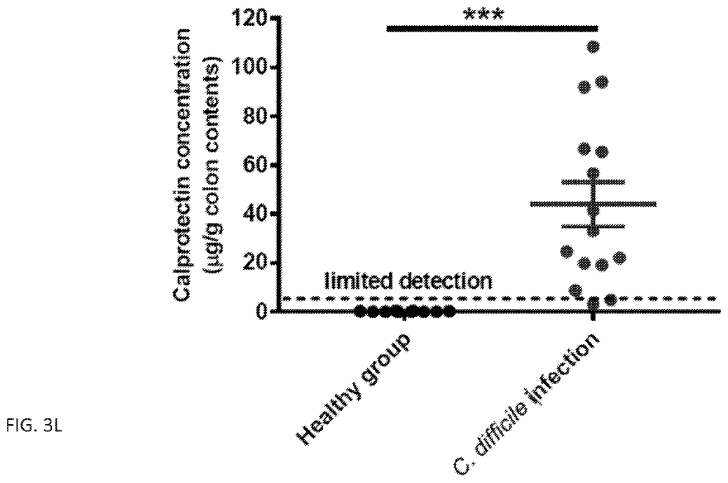

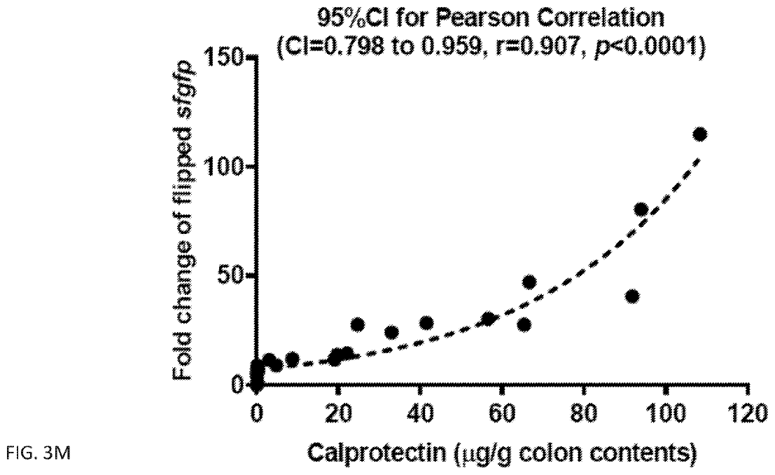

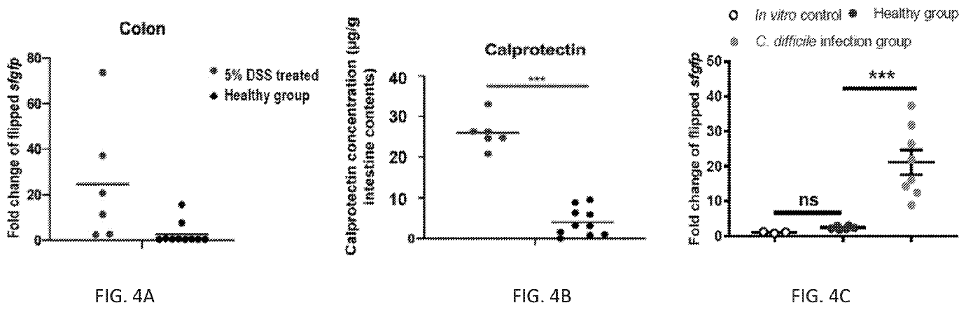

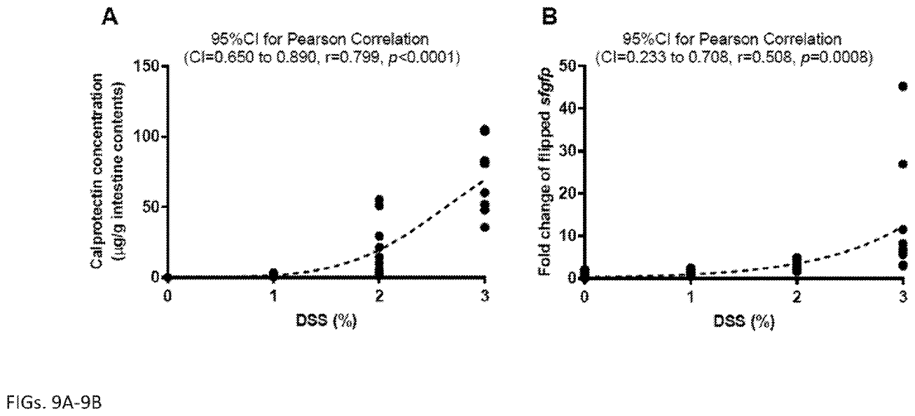

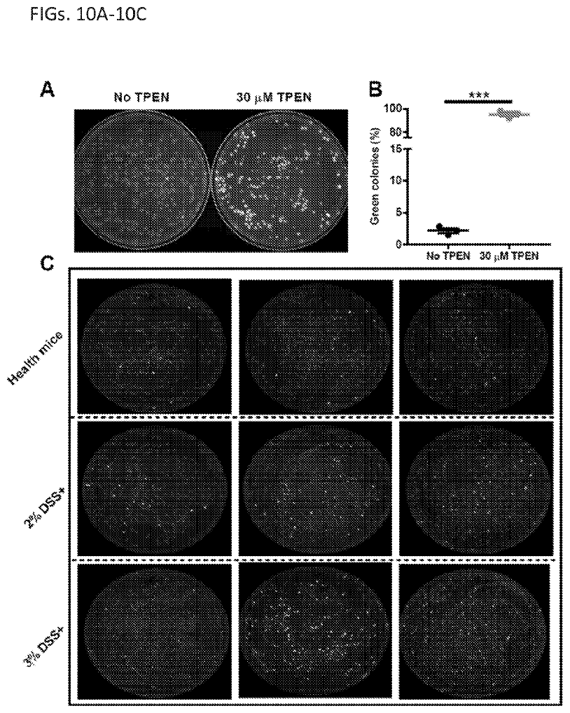

Genetic memory circuit biosensor senses intestinal inflammation in vivo. To test if the BSIM would be able to sense and accurately report inflammation in the context of the intestinal tract, the biosensor was tested in two animal models of inflammation. In the first model, the mice were treated with dextran sodium sulfate (DSS), a chemical agent used to induce inflammatory colitis in mice. Animals were treated with DSS in their drinking water for 7 days and clinical symptoms (weight loss, stool change) were observed. The BSIM biosensor was gavaged intragastrically into animals on day 6 and colons and luminal contents were collected four hours later (FIGS. 3A-B). To quantify activation of BSIM in the gut, qPCR was performed with primers that measure the flipped orientation of sfgfp (BSIM on) in colon contents. BSIM was first tested in vivo using mice that had been exposed to a high dose (5%) of DSS and demonstrated strong activation of BSIM and increased calprotectin level in the DSS treated mice compared to control animals (FIG. 4A-B). To evaluate the sensitivity of BSIM, mice were treated with different levels of DSS (1-3%) in drinking water. Treating mice with 3% DSS caused significant weight loss (FIG. 3C), while both 2 and 3% DSS caused shortening of colon length (FIG. 3D) and increased calprotectin levels in the colon (FIG. 3E). Treatment with 1% DSS showed no overt clinical symptoms. Mice treated with 3% DSS showed a 10-fold increase in BSIM activation (range 3-45 fold, p<0.0001) while healthy control mice and mice treated with 1% DSS showed no significant induction of BSIM (FIG. 3F). Mice treated with 2% DSS displayed a trend towards increased BSIM activation (3-fold increase, range 2-5 fold, p=0.0062). Meanwhile, both calprotectin levels and sensor activation were correlated with DSS concentration (FIGS. 9A-9B). To evaluate the percentage of cells in which BSIM was activated in the mice, colon contents were plated on LB medium with ABX to select cells harboring BSIM and then counted the number of cells displaying green fluorescence by microscopy. A 4 fold increase in the number of activated BSIM was found in 3% DSS treated mice compared to that of the healthy mice, with an intermediate activation in 2% DSS observed (FIG. 3H and FIG. 9).

To further validate the utility of BSIM for detecting inflammation in vivo, a second model was chose that instead models intestinal inflammation that was based on an infectious agent, Clostridoides difficile, which has been shown to induce a large infiltration of neutrophils during infection.31 Animals were treated with a regimen of antibiotics followed by a single intraperitoneal injection of clindamycin prior to infection with C. difficile VPI10463 (ribotype 087, high toxin producer) (FIG. 4C) or C. difficile R20291 (RT027) (FIG. 4I-M) that causes a moderate to severe infection under these conditions (FIG. 3I). As expected, animals showed weight loss and clinical symptoms of disease within 48 hours (FIG. 3J). The memory biosensor BSIM was then orally delivered into animals after which colon contents were collected 4 hours later. As in the DSS colitis experiments, strong activation (9-115 fold) of BSIM occurred in the C. difficile-treated animals while it remained off or lowly expressed in healthy controls (FIG. 3K). As observed in the DSS study, BSIM activation correlated with increased levels of calprotectin in the intestine (FIGS. 3L-3M).

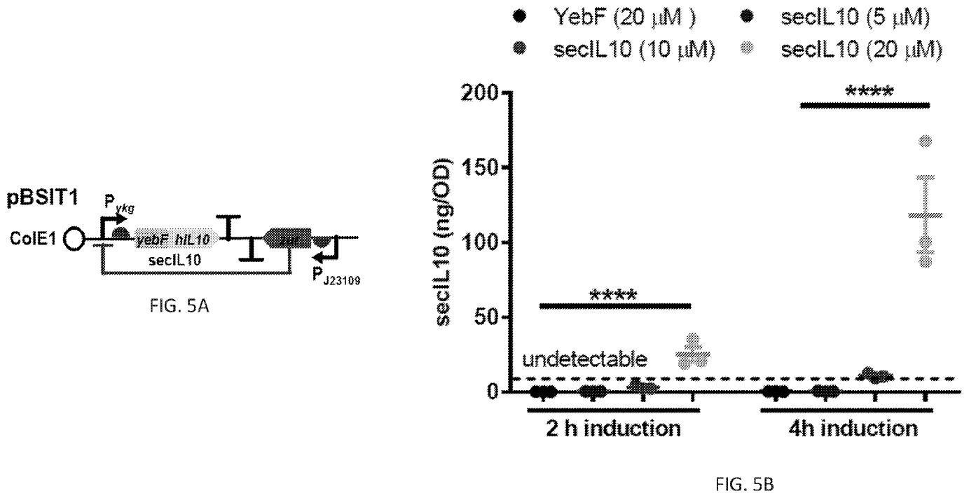

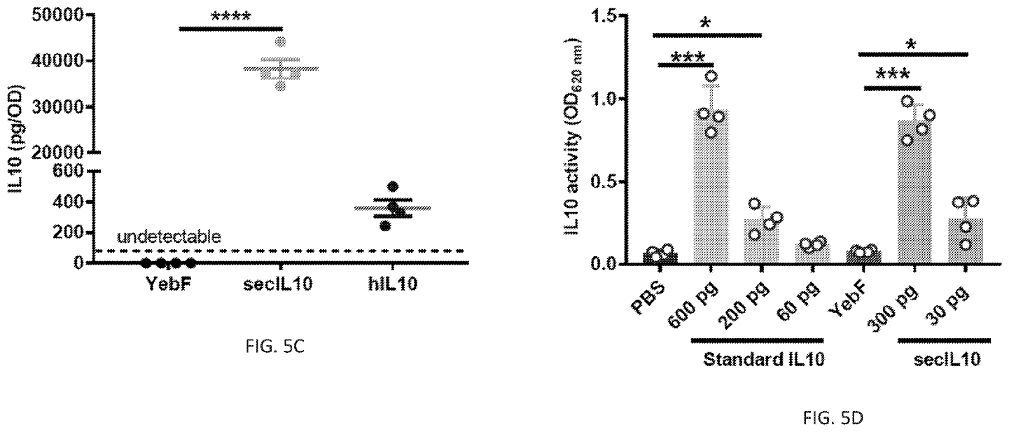

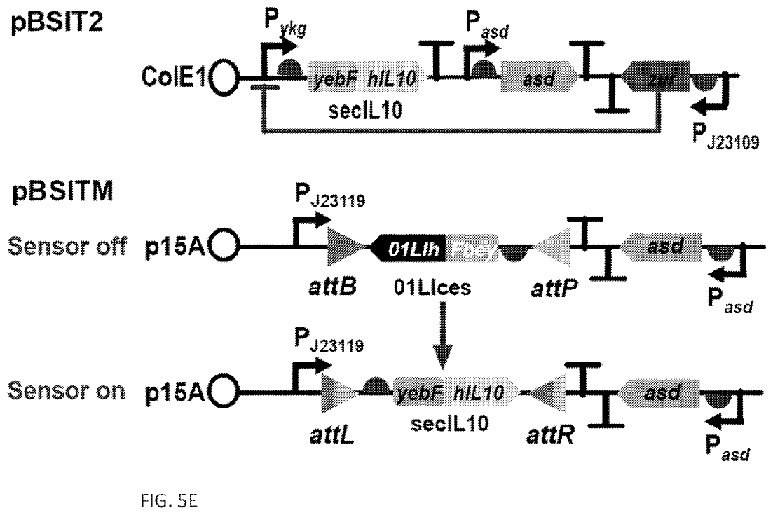

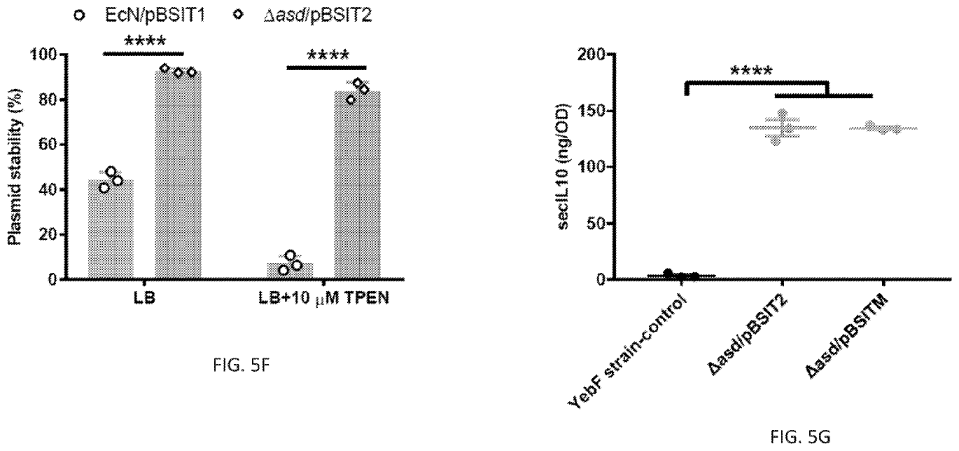

Secretion of functionally active human IL10 (hIL-10) in response to inflammation. hIL-10 has potent anti-inflammatory activity and has been used to successfully ameliorate intestinal inflammation in animal models of colitis. However, difficulties in secreting large proteins from EcN made it necessary to devise a system to have sufficient secretion of hIL-10 to ameliorate disease. Previously it was shown that the YebF protein can be used to facilitate the secretion of proteins into the extracellular medium²⁷. sfgfp was replaced with a fusion gene yebl-hll.10 (designated secIL 10) and yebl′ only, resulting in the secIL 10 expression plasmid pBSIT1 (FIG. 5A) and yebl′ control pBSITC1, respectively. The ability of E. coli Nissle (EcN) carrying pBSIT1 was tested for production and secretion of secIL 10 in LB growth medium in the presence of 5-20 μM of TPEN, EcN/pBSITC1 (BSITC1) served as a control. It was observed that 119 ng/OD of secIL 10 was secreted into the culture when 20 μM TPEN was added after 4 h induction (FIG. 5B). This data showed a significant higher concentration of secIL 10 in BSIT1 compared to YebF control and leaking expression construct (FIG. 5C). These data showed that the biosensor can be used to induce and secrete the anti-inflammatory IL-10 cytokine when fused to YebF.

To test if the secIL-10 secreted into the supernatant was functionally active as a fusion protein BSIT1 was induced with 20 μM TPEN for 3 h and conditioned supernatant was tested for IL-10 activity using an IL-10 reporter cell line. The secreted IL-10 from BSIT1 activated the reporter as strongly as recombinant IL-10 (FIG. 5D). The data showed that the engineered therapeutic biosensor BSIT1 can secrete active IL-10.

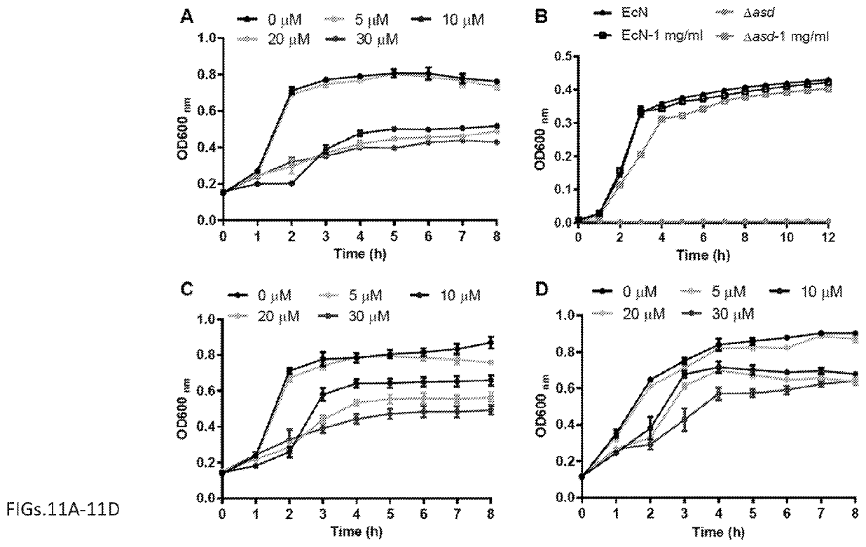

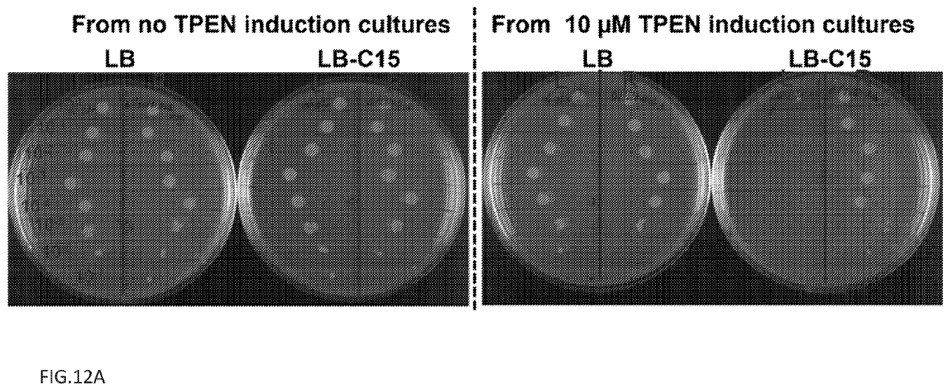

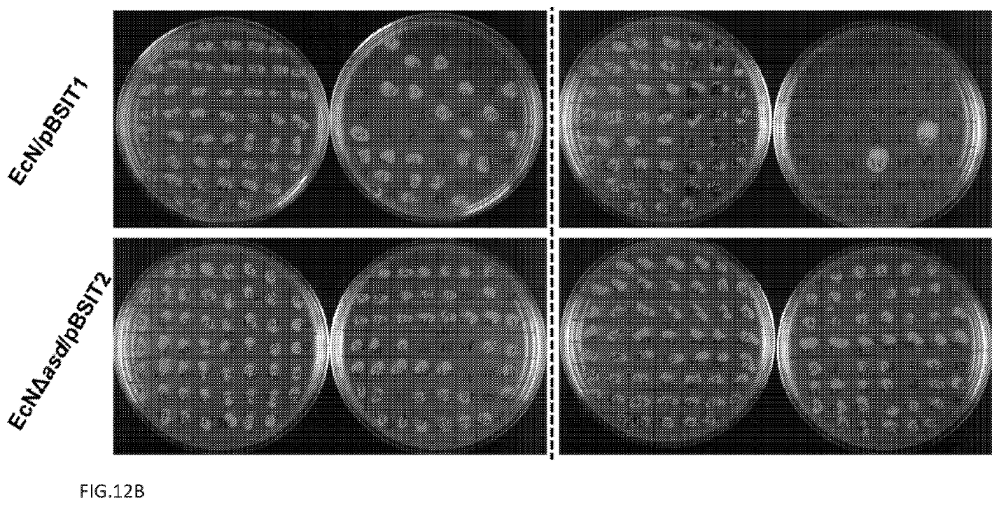

To ensure that pBSIT1 would remain stably associated with EcN in the absence of antibiotic selection, an existing strategy was used for making the presence of pBSIT1 essential for EcN³² growth. An essential gene (asd) that is required in lysine, threonine, and methionine biosynthesis was deleted in the EcN genome and was complemented in sensor plasmid pBSIT (pBSIT2) (FIG. 5E). The asd mutant strain harboring plasmid pBSITC2 (BSITC2) which only contains yebl was used as a control. The data confirmed that EcNΔasd cannot grow without the essential substrate diaminopimelic acid (DAP) added to the growth medium (FIG. 11B). The therapeutic biosensor EcNΔasd/pBSIT2 (BSIT2) exhibited improved stability after TPEN induction compared to BSIT1 (FIG. 5F, generated from FIG. 12). EcNΔasd/pBSIT2 (BSIT2) showed a similar growth profile to EcN/pBSIT1 (BSIT1) (FIGS. 11C-11D) and displayed similar secIL 10 production in induced supernatant compared to BSIT1 (FIG. 5G).

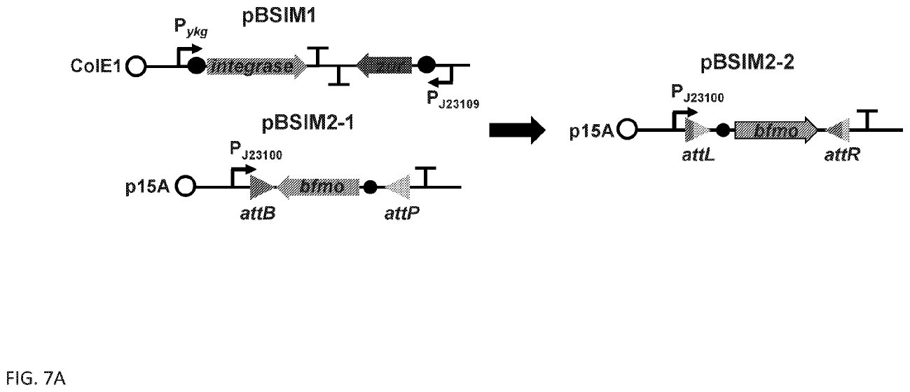

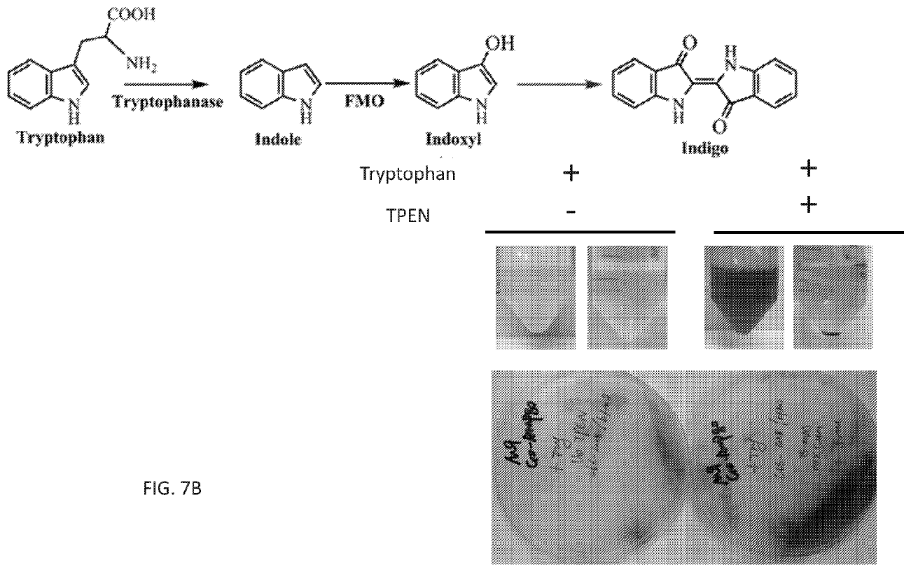

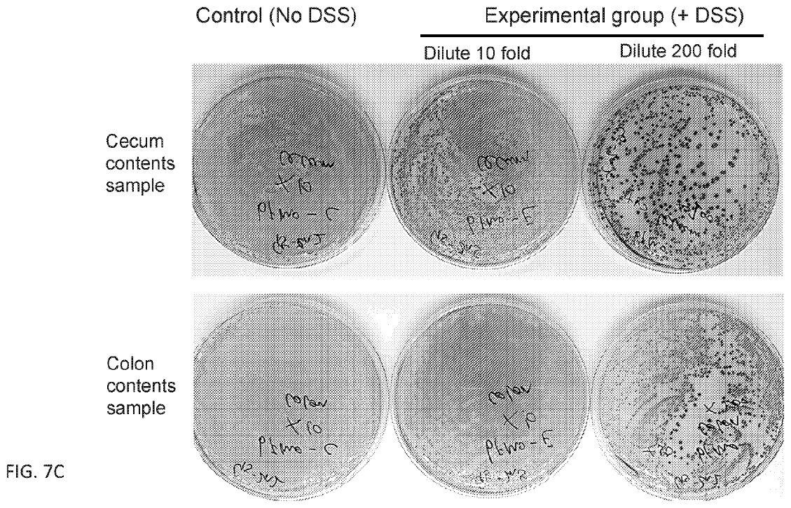

To achieve identification of the gut inflammation by visible pools color change, the sfgfp gene was replaced with the bfmo gene in the pBSIM2-1 plasmid (denoted pBSIM2-bfmo), which can transform tryptophan to indigo and produce blue pools from an inflamed gut (FIG. 7A). The bfmo-based memory circuit biosensor was tested in vitro, as shown in FIG. 7B. When TPEN and tryptophan were added to culture media, the biosensor can outcome a visible deep blue color both in liquid cultures and agar plates (FIG. 7B). To test the activity of the bfmo biosensor in vivo, the bfmo biosensor was gavaged into DSS-treated mice, and feces and colon contents were collected. Then, the collected samples were diluted and plated on culture plates with tryptophan added. Results showed that more blue colonies can be found in the DSS-treated mice sample (FIG. 7C). The data showed that the bfmo gene-based biosensor can be activated in the inflamed gut and used for gut inflammation detection.

### Therapeutic Biosensor Ameliorates Intestinal Inflammation in Vivo.

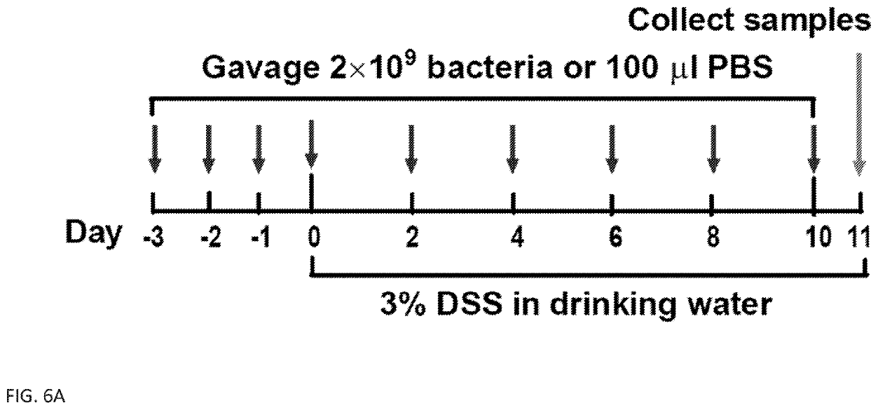

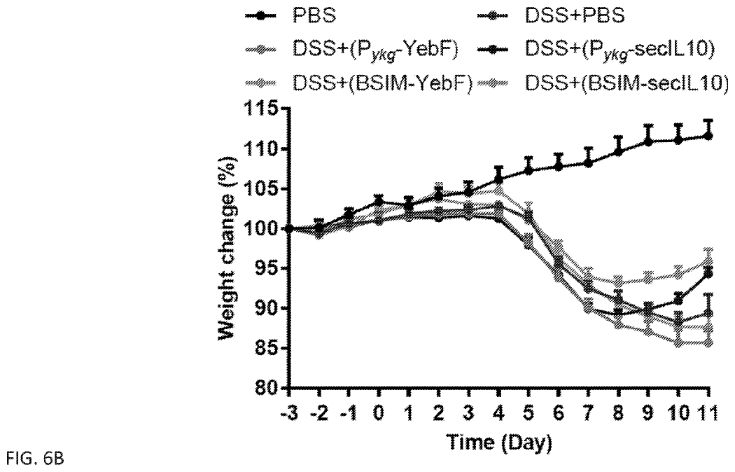

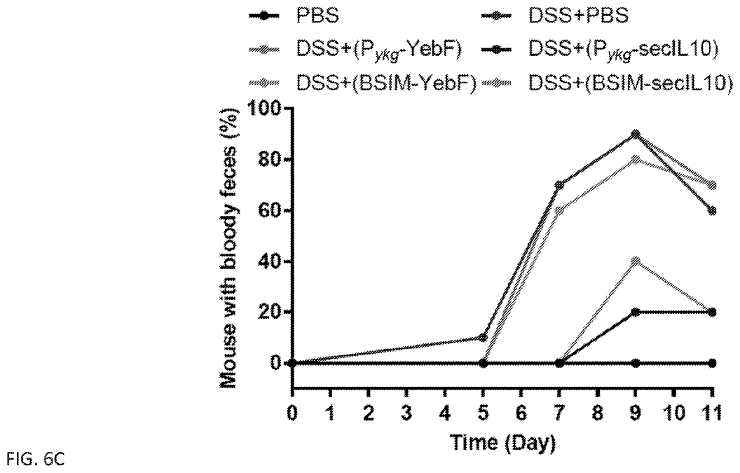

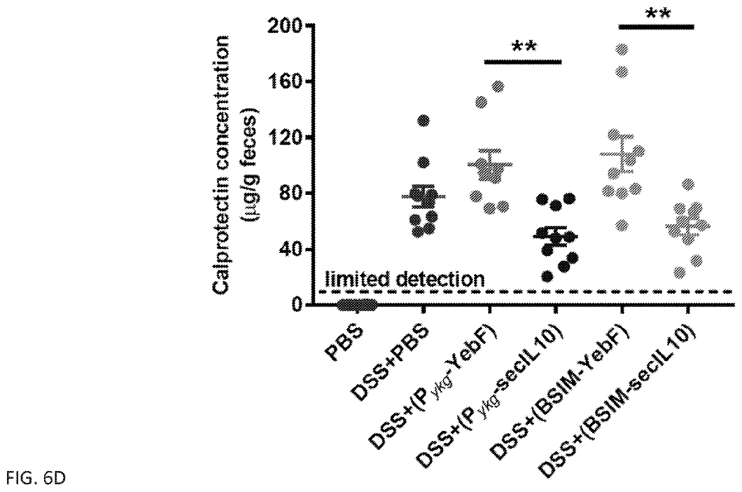

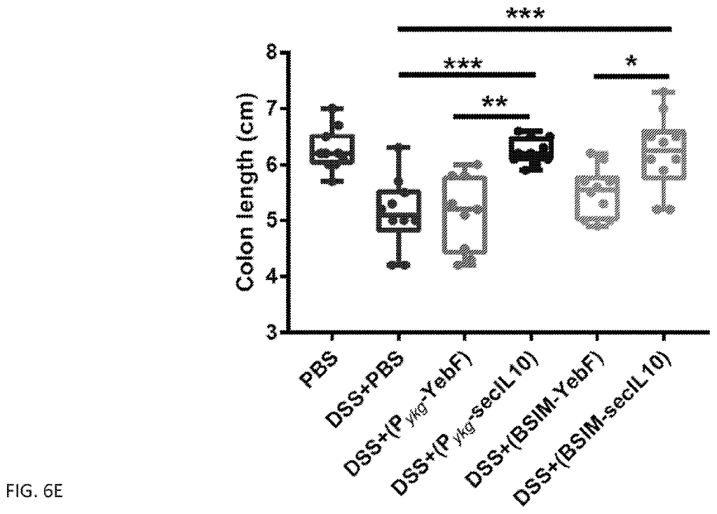

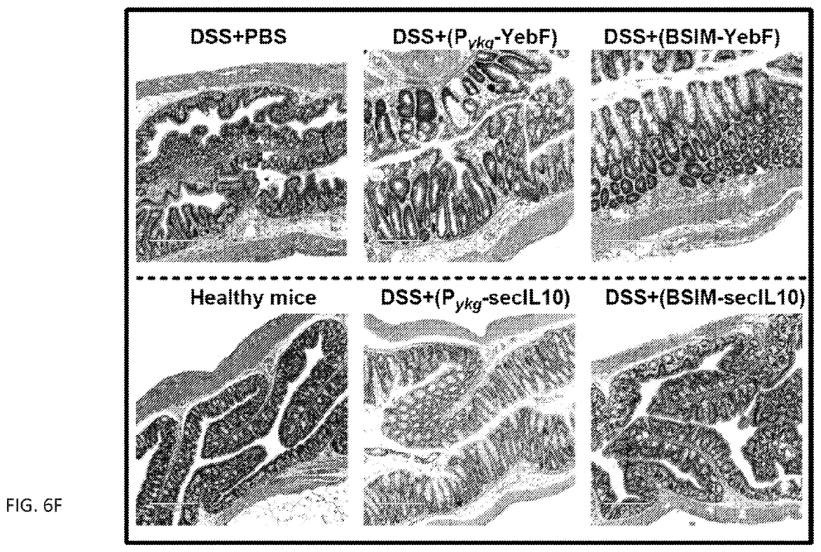

To evaluate the ability of the therapeutic biosensor to reduce inflammation in vivo in response to sensing inflammation, an intragastric inocula of 2×10⁹ CFU of BSIT2 (Δasd/pBSIT2) was applied to 3% DSS treated mice (FIG. 6A). A significant weight change was not observed between secIL10 treated groups and control groups (DSS and DSS+YebF) (FIG. 6B), but BSIT2 displayed fewer mice with bloody feces and delayed inflammation compared to control groups (FIG. 6C). The fecal calprotectin concentration of BSIT2 was significantly lower than that in the control groups, indicating a lower level of inflammation in the BSIT2 treated animals (FIG. 6D). Histological score analysis was performed for the distal colon on collected colons. The colon length of BSIT2 treated mice was significantly longer than DSS+YebF and DSS+PBS treated mice and similar to the healthy mice (FIG. 6E). More goblet cells and crypt damage was also observed along with a higher infiltration of inflammatory cells into the mucosa in controls vs BSIT2 treated mice. (FIG. 6F). The lower level of the histological score of BSIT2 treated mice demonstrated the amelioration of inflammation with secIL 10 being regulated by the inflammation biosensor. The data showed the inflammation biosensor can be actively coupled with inflammation amelioration in vivo.

### Significance of Certain Embodiments

The human intestinal tract is a single cell layer epithelium that separates the body from the outside world and controls what is absorbed and repelled, contributes to the immune response, and keeps indigenous and ingested microbes at bay. Inflammatory bowel disease, consisting of Crohn's disease and Ulcerative colitis, is a chronic inflammation of the intestine that undergoes cycles of flares and remission. Management of IBD is challenging, because there are no available tests that can be performed non-invasively and that do not require patients to handle their feces. The present disclosure concerns development of a bacterial biosensor that accurately distinguishes between healthy and inflamed mice in two independent mouse models of colitis.

A number of studies have shown that newly-identified biomarkers, including thiosulfate, tetrathionate, pH, and reactive oxygen species, are candidates for using synthetic microbes to sense inflammation. However, most of these biomarkers are currently not validated for the ability to diagnose intestinal inflammation. Calprotectin-inducible promoters in E. coli Nissle are utilized herein because of calprotectin's well-defined role in nutritional immunity and because fecal calprotectin is the most widely accepted test for monitoring intestinal inflammation. It was considered that E. coli has evolved regulons to address metal limitation imposed by calprotectin released by neutrophils in the gut during inflammation. Because strains of E. coli have had to exist and compete in the intestinal environment during co-evolution with humans, it seemed likely that promoters responding to calprotectin release by neutrophils would function in a physiological range of inflammation.

The ykgMO operon is a well-studied gene cluster that responds to zinc limitation by expressing YkgM and YkgO when zinc concentrations fall to levels that threaten cell growth. YkgM and YkgO encode alternative large ribosomal subunit proteins L31 and L36, respectively. Under zinc replete conditions the normal versions of L31 and L36 contain zinc molecules that are required for their function. The YkgM (L31) and YkgO (L36) proteins do not require zinc to perform their functions in the ribosome and allow translation to continue under conditions of zinc starvation. Thus, the ykgMO) promoter was an excellent candidate promoter for the optimization of a biosensor because it is highly expressed when repression by Zur, is relieved by zinc limitation, and it was relatively simple to engineer a low basal level of expression of the promoter by regulating the level of Zur in the cell. This was validated by the identification of the operon as being induced by calprotectin in vitro as well as being able to distinguish inflamed versus healthy guts in two independent animal models.

### EXAMPLES OF MATERIALS AND METHODS

### Bacterial Strains and Media

All strains used are listed in Table 2. Escherichia coli Nissle 1917 (referred hereafter as EcN) was grown aerobically at 37° C. using either Luria-Bertani (LB) broth (1% tryptone, 0.5% yeast extract, 1% NaCl) or the minimal defined media M9 (200 ml 5×M9 salts+0.4% glucose+2 mM MgSO₄+0.1 mM CaCl₂+0.2% casamino acids for 1 liter M9 media). Escherichia coli EC1000 was used as an electroporation cloning host. Chloramphenicol (15 μg/mL) and ampicillin (100 μg/mL) were used for E. coli selection when needed. Clostridioides difficile R20291 was cultured in BHIS media (brain heart infusion broth supplemented with 0.5% yeast extract and 0.1% L-cysteine, and 1.5% agar for agar plates) at 37° C. in an anaerobic chamber (90% N₂, 5% H₂, 5% CO₂). For spores preparation, C. difficile strains were cultured in Clospore media and purified.

TABLE 2
Bacteria and plasmids utilized in this disclosure

| Strains or plasmids | Genotype or phenotype | Reference |
|---|---|---|
| Strains | | |
| E. coli DH5α | Cloning host | NEB |
| E. coli Nissle1917 | Engineered biosensor host | Lab stock |
| (EcN) | | |
| EcNΔasd | Essential gene asd deleted in EcN | This work |
| BSI0 | EcN containing pBSI0 | This work |
| BSI1 | EcN containing pBSI1 | This work |
| BSIM | EcN containing pBSIM1 and | This work |
| | pBSIM2 | |
| BSIM-bfmo | EcN containing pBSIM1 and | This work |
| | pBSIM2-bfmo | |
| BSIT0 | EcN containing pBSIT0 | This work |
| BSIT1 | EcN containing pBSIT1 | This work |
| BSIT2 | EcNΔasd containing pBSIT2 | This work |
| BSIT3 | EcNΔasd containing pBSIT3 | This work |
| C. difficile R20291 | Clinical isolate; ribotype 027 | Lab stock |
| C. difficile VPI10463 | Clinical isolate; ribotype 078 | Lab stock |
| Plasmids | | |
| pBSI0 | pColE-P_{ykg} -sfgfp | This work |
| pBSI0-Pₑₙₜ | pColE-Pₑₙₜ -sfgfp | This work |
| pBSI0-P_{abt} | pColE-P_{abt}-sfgfp | This work |
| pBSI1 | pColE-P_{ykg} -sfgfp-P_{J23109}-zur | This work |
| pBSIM1 | pColE-P_{ykg} -int8-P_{J23109}-zur | This work |
| pBSIM2 | p15A- P_{J23119}-rsfgfp-P_{J23109}-zur | This work |
| pBSIM2-bfmo | p15A- P_{J23119}-rbfmo-P_{J23109}-zur | This work |
| pBSIT0 | pColE-P_{ykg} -yebF-P_{J23109}-zur | This work |
| pBSIT1 | pColE-P_{ykg} -secIL10-P_{J23109}-zur | This work |
| pBSIT2 | pColE-P_{ykg} -secIL10-P_{asd}-asd- | This work |
| | P_{J23109}-zur | |
| pBSITM | p15A-P_{J23119}-rsecIL10-P_{asd}-asd- | This work |
| | P_{J23109}-zur | |

### Biosensor Promoters Identification

To explore biosensor promoters that can respond to calprotectin, the transcriptome of EcN treated with the sub-inhibitory amount of calprotectin was analyzed. To detect the minimum inhibitory concentration (MIC) of calprotectin, EcN was grown overnight in LB broth. From the overnight culture, 10⁴ cells in 38 μL of LB broth were seeded into a 96 well-plate. 62 μL of calprotectin in buffer (20 mM Tris, 100 mM NaCl, 10 mM beta-mercaptoethanol, 3 mM CaCl₂)) was added in 2-fold dilutions. Recombinant human calprotectin was supplied by Dr. Walter Chazin of Vanderbilt University. OD600 values were taken after 16-18 hours of growth.

For transcriptome analysis, EcN cells were grown overnight, and then back-diluted 1:100 into 5 mL of LB broth. When cells were grown up to the log phase, the sub-inhibitory amount of calprotectin was added to the cultures at a final concentration of 125 μg/mL. Growth continued for 30 min in the presence of calprotectin, and then transcription was frozen by the addition of ice-cold ethanol. Bacteria were collected via centrifugation, and RNA was isolated using the QIAgen RNEasy kit (Qiagen, Hilden, Germany). Slight modifications were included: lysozyme/proteinase K was used to disrupt the EcN cell wall. RNA was quantified and tested for purity using a Denovix spectrophotometer (Denovix, Wilmington, Delaware). RNA-seq was performed by Applied Biological Materials (ABM) (Richmond, British Columbia, Canada) on an Illumina NextSeq sequencer, at an average of 5 million reads per sample. rRNA depletion and quality check were also performed by ABM. Sequences were received from ABM in fastq format. Raw sequence filtering and alignment were performed by the Center for Metagenomics and Microbiome Research at Baylor College of Medicine. Sequences were aligned against EcN reference genome. Raw counts were obtained and gene expression analysis was performed using DESeq2 on R-Studio. Genes that had at least a 2-fold increase in expression in the presence of calprotectin were passed for construct engineering.

### Biosensor Plasmids Construction

Genomic DNA was isolated and promoter regions were amplified via PCR. The pColE1 plasmid was used for the biosensor construction. Initial test constructs consisted of the intergenic promoter regions for the following genes: ent (′H (enterobactin synthase operon), ykgM ( ) (paralog for L31/L36 ribosomal accessory protein), ABC Transporter (WP_000977398.1). Each promoter region was cloned directly upstream of a super fold green fluorescent protein cassette (sfgfp). All constructs were assembled via the Gibson Reaction (New England Biosciences, Ipswich, Massachusetts). The resultant plasmids were named as pColE1-Pₑₙₜ-sfGFP, pColE1-Pykg-sfgfp, and pColE1-Pabt-sfgfp, respectively. All Gibson oligos were made using IDT, and all amplicons were synthesized with Phusion polymerase (NEBiosciences, Ipswich, Massachusetts). A listing of all constructed plasmids used in this study can be found in Table 2.

### Flow Cytometry and Data Analysis

EcN biosensor constructs were grown to early log phase (˜0.1 OD600) in M9 media with glucose, and were back diluted to 0.01 OD600 and then grown for ˜4 hours aerobically, until OD600 reached ˜0.15. At this point, cells were kept on ice until analysis on a Becton Dickinson FACScan flow cytometer. Flow cytometry runs were performed in 96 well plates. Briefly, 10-40 μL of cells were added to 1 mL of PBS sheath fluid and run through the flow cytometer for a total of 10000 events. Cells were thresholded on forward/side scatter, and .fsc files were analyzed with the FlowCal software. In short, FlowCal identifies the densest region of cells on the associated scatterplot, and analyzes the fluorescent output of 30% of the cells in this region so as to evaluate a homogenous dataset and remove possible outliers. This results in a geometric mean of total fluorescent output. In this case, sfGFP output is reported as molecules of equivalent fluorophores (MEF), and was evaluated on the FL1 channel. More information on FlowCal can be found at: http://taborlab.github.io/FlowCal/.

### Calprotectin and TPEN Induction and Metal Complementation

Final concentrations of 40 μg/mL recombinant human calprotectin and 1.5, 3, or 30 μM TPEN were used for the biososensor constructs induction test, respectively. The following mix was added to each well-124 μL of recombinant human calprotectin in calprotectin buffer, 56 μL of M9 media, and 20 μL of cells (10+). TPEN (Sigma Aldrich, St. Louis, MO), a synthetic metal chelator, induction assays were performed as calprotectin induction, with the following differences-160 μL of M9 media, 20 μL of cells (10+), and 20 μL of either 15, 30, or 300 μM TPEN was added to each well. TPEN was dissolved in absolute ethanol. The absolute ethanol was used as a negative control.

Mixes were similar to the calprotectin induction assays with the addition of zinc sulfate, manganese sulfate, or iron chloride. Concentrations of metals were added in excess of 1× and 10× the binding capacity of 40 μg/mL (1.5 μM) calprotectin. In total, 4 μM and 40 μM of zinc and iron were added, and 2 μM and 20 μM of manganese was added.

### Optimization of Biosensor

To reduce the background of Pygk biosensor, zinc binding transcription factor Zur was inserted into pColE-based sensor construct (pColE1-Pykg-sfgfp) under the control of different constitutive expresison promoters. Among them, five different constitutive promoters J23100 (P1), J12110 (P2), J121140 (P3), J23109 (P4), and J23113 (P5) from Registry of Standard Biological Parts (http://parts.igem.org/Promoters/Catalog/Anderson) were selected and tested. The sensitivity and expression strength of new biosensor constructs was analyzed through fluorescence detection and RT-qPCR.

Because it was expected that zinc sequestration to levels that will activate the biosensor will only occur at sites of active inflammation with neutrophil infiltration, it was considered that the ideal biosensor would need to become permanently activated upon sensing inflammation for future readout in the stool. A memory switch biosensor was developed containing a two-plasmid system using phage integrase 8 being driven by the P_{ykg} promoter regulated by Zur on one plasmid (pBSIM1) and a sfGFP gene on the second plasmid in the opposite orientation of the strong promoter P1 (J23119) (denoted pBSIM2). The sfgfp gene is flanked by att sites that are recognized by the integrase and when expressed will flip the orientation of the sfgfp gene to allow expression from promoter P1.

### Therapeutic Biosensor Construction

To engineer secreted IL 10 expression EcN strain, YebF, a facilitating carrier protein was fused up to IL10 (YebF-IL10, designated secIL 10) and assembled into pColE1 based plasmid pBSI1 which replaced sfgfp with secIL10 (denoted as pBSIT1). pBSIT1 was then transformed into EcN (denoted as EcN/BSIT1). To achive stable expression of secIL 10, an essential gene asd in EcN was deleted by CRISPR-Cas9 and complemented asd gene in the therapeutic sensor plasmid, named as pBST2. Meanwhile, the sfgfp gene in pBSIM2 was also replaced with secIL10 (named as pBSITM) to get the therapeutic biosensor with memory circuit. Supernatants of therapeutic biosensor with TPEN induced were analyzed by IL10 ELISA kit. The activity of secIL10 was analyzed by the Human&Murine IL-10 reporter cells (HEK-Blue™ IL10 Cells) that is engineered to respond to functional IL-10 according to instruction.

### Animal Experiments

For dextran sodium sulfate (DSS, MW=40,000, Thermos Scientific) induced IBD mouse model, six-week-old female and male C57BL/6 mice were procured from Baylor College of Medicine in Houston, Texas. Mice were transferred to an established protocol that was approved by the Baylor College of Medicine Institutional Animal Care and Use Committee (IACUC). Mice were treated with or without 5% (w/v) DSS in drinking water for 5 days. On the fifth day, mice were gavaged with 10⁹ CFU of EcN biosensor. 4-6 hours after gavage, fecal pellets and colon contents from all mice were collected. The total DNA from stool and colon contents were isolated by E.Z.N.A Stool DNA kit (Omega) according to the instruction for biosensor activation efficiency analysis by qPCR.

To test the biosensor in the Clostridioides difficile infection mouse model (CDI), six-week-old female and male C57BL/6 mice were given an orally administered antibiotic cocktail (kanamycin 0.4 mg ml-1, gentamicin 0.035 mg ml-1, colistin 0.042 mg ml-1, metronidazole 0.215 mg ml-1, and vancomycin 0.045 mg ml-1) in drinking water for 4 days. After 4 days of antibiotic treatment, all mice were given autoclaved water for 2 days, followed by one dose of clindamycin (10 mg kg-1, intraperitoneal route) 24 h before spores challenge (Day 0). After that, mice were orally gavaged with either 10⁴ or 10⁵ of spores or PBS as a control. 2 days after spores gavage, 10⁹ CFU of EcN biosensor were gavaged to all mice. 4-6 hours after biosensor gavage, fecal pellets (if possible) and colon contents were collected for biosensor activation test.

To test the therapeutic biosensor for inflammation amelioration in vivo, the IBD animal model was used. Six-week-old male C57BL/6 mice were purchased. Before DSS treatment, mice were gavaged with PBS (control group and +DSS group or sensor constructs for 3 days. Following, the mice were treated with or without 3% (w/v) DSS in drinking water and orally gavaged with PBS or engineered EcN constructs every two days for 10 days. The mice weight and disease severity was monitored every day. On the eleventh day, colon and colon contents from all mice were collected.

### REFERENCES

1 McDowell, C. & Haseeb, M. Inflammatory bowel disease (IBD). (2017).
2 Ye, Y., Manne, S., Treem, W. R. & Bennett, D. Prevalence of Inflammatory Bowel Disease in Pediatric and Adult Populations: Recent Estimates From Large National Databases in the United States, 2007-2016. Inflamm Bowel Dis 26, 619-625, doi: 10.1093/ibd/izz182 (2020).
3 Kaplan, G. G. The global burden of IBD: from 2015 to 2025. Nat Rev Gastroenterol Hepatol 12, 720-727, doi: 10.1038/nrgastro.2015.150 (2015).
4 Singh, S. et al. Trends in U.S. Health Care Spending on Inflammatory Bowel Diseases, 1996-2016. Inflamm Bowel Dis 28, 364-372, doi: 10.1093/ibd/izab074 (2022).
5 Gupta, N., Renugopalakrishnan, V., Liepmann, D., Paulmurugan, R. & Malhotra, B. D. Cell-based biosensors: Recent trends, challenges and future perspectives. Biosens Bioelectron 141, 111435, doi: 10.1016/j.bios.2019.111435 (2019).
6 Pickard, J. M. et al. Rapid fucosylation of intestinal epithelium sustains host-commensal symbiosis in sickness. Nature 514, 638-641, doi: 10.1038/nature13823 (2014).
7 Kotula, J. W. et al. Programmable bacteria detect and record an environmental signal in the mammalian gut. Proc Natl Acad Sci USA 111, 4838-4843, doi: 10.1073/pnas. 1321321111 (2014).
8 Drouault, S., Anba, J. & Corthier, G. Streptococcus thermophilus is able to produce a beta-galactosidase active during its transit in the digestive tract of germ-free mice. Appl Environ Microbiol 68, 938-941, doi: 10.1128/AEM.68.2.938-941.2002 (2002).
9 Mimee, M., Tucker, A. C., Voigt, C. A. & Lu, T. K. Programming a Human Commensal Bacterium, Bacteroides thetaiotaomicron, to Sense and Respond to Stimuli in the Murine Gut Microbiota. Cell Syst 2, 214, doi: 10.1016/j.cels.2016.03.007 (2016).
10 Daeffler, K. N. et al. Engineering bacterial thiosulfate and tetrathionate sensors for detecting gut inflammation. Mol Syst Biol 13, 923, doi: 10.15252/msb.20167416 (2017).
11 Nammi, D. & Neelapu, N. R. R. in Recent Advancements in Biomarkers and Early Detection of Gastrointestinal Cancers 133-168 (Springer, 2020).
12 Walsham, N. E. & Sherwood, R. A. Fecal calprotectin in inflammatory bowel disease. Clin Exp Gastroenterol 9, 21-29, doi: 10.2147/CEG.S51902 (2016).
13 Ricciuto, A. & Griffiths, A. M. Clinical value of fecal calprotectin. Crit Rev Clin Lab Sci 56, 307-320, doi: 10.1080/10408363.2019.1619159 (2019).
14 Vogl, T., Leukert, N., Barczyk, K., Strupat, K. & Roth, J. Biophysical characterization of S100A8 and S100A9 in the absence and presence of bivalent cations. Biochim Biophys Acta 1763, 1298-1306, doi: 10.1016/j.bbamcr.2006.08.028 (2006).
15 Fagerhol, M., Andersson, K., Naess-Andresen, C., Brandtzaeg, P. & Dale, I. J. B. R., Fla: CRC Press, Inc. Calprotectin (the L1 leukocyte protein) In: Smith VL, Dedman JR, editors. Stimulus response coupling: the role of intracellular calcium-binding proteins. 187-210 (1990).
16 Brophy, M. B., Hayden, J. A. & Nolan, E. M. Calcium ion gradients modulate the zinc affinity and antibacterial activity of human calprotectin. J Am Chem Soc 134, 18089-18100, doi: 10.1021/ja307974e (2012).
17 Nakashige, T. G., Zhang, B., Krebs, C. & Nolan, E. M. J. N. c. b. Human calprotectin is an iron-sequestering host-defense protein. 11, 765-771 (2015).
18 Nakashige, T. G., Zygiel, E. M., Drennan, C. L. & Nolan, E. M. Nickel Sequestration by the Host-Defense Protein Human Calprotectin. J Am Chem Soc 139, 8828-8836, doi: 10.1021/jacs. 7601212 (2017).
19 Zhou, J. et al. Programmable probiotics modulate inflammation and gut microbiota for inflammatory bowel disease treatment after effective oral delivery. Nat Commun 13, 3432, doi: 10.1038/s41467-022-31171-0 (2022).
20 Riglar, D. T. & Silver, P. A. Engineering bacteria for diagnostic and therapeutic applications. Nat Rev Microbiol 16, 214-225, doi: 10.1038/nrmicro.2017.172 (2018).
21 Aggarwal, N., Breedon, A. M. E., Davis, C. M., Hwang, I. Y. & Chang, M. W. Engineering probiotics for therapeutic applications: recent examples and translational outlook. Curr Opin Biotechnol 65, 171-179, doi: 10.1016/j.copbio.2020.02.016 (2020).
22 Li, M. C. & He, S. H. IL-10 and its related cytokines for treatment of inflammatory bowel disease. World J Gastroenterol 10, 620-625, doi: 10.3748/wjg.v10.i5.620 (2004).
23 Steidler, L. et al. Treatment of murine colitis by Lactococcus lactis secreting interleukin-10. Science 289, 1352-1355 (2000).
24 Watterlot, L. et al. Intragastric administration of a superoxide dismutase-producing recombinant Lactobacillus casei BL23 strain attenuates DSS colitis in mice. Int J Food Microbiol 144, 35-41, doi: 10.1016/j.ijfoodmicro.2010.03.037 (2010).
25 Whelan, R. A. et al. A transgenic probiotic secreting a parasite immunomodulator for site-directed treatment of gut inflammation. Mol Ther 22, 1730-1740, doi: 10.1038/mt.2014.125 (2014).
26 Fioranelli, M. & Roccia, M. G. J. J. I. C. Twenty-five years of studies and trials for the therapeutic application of IL-10 immunomodulating properties. From high doses administration to low dose medicine new paradigm. 1, 2-6 (2014).
27 Zhang, G., Brokx, S. & Weiner, J. H. Extracellular accumulation of recombinant proteins fused to the carrier protein YebF in Escherichia coli. Nat Biotechnol 24, 100-10⁴, doi: 10.1038/nbt1174 (2006).
28 Gabriel, S. E. & Helmann, J. D. Contributions of Zur-controlled ribosomal proteins to growth under zinc starvation conditions. J Bacteriol 191, 6116-6122, doi: 10.1128/JB.00802-09 (2009).
29 Liu, J. Z. et al. Zinc sequestration by the neutrophil protein calprotectin enhances Salmonella growth in the inflamed gut. Cell Host Microbe 11, 227-239, doi: 10.1016/j.chom.2012.01.017 (2012).
30 Kehl-Fie, T. E. et al. Nutrient metal sequestration by calprotectin inhibits bacterial superoxide defense, enhancing neutrophil killing of Staphylococcus aureus. Cell Host Microbe 10, 158-164, doi: 10.1016/j.chom.2011.07.004 (2011).
31 Jose, S. & Madan, R. Neutrophil-mediated inflammation in the pathogenesis of Clostridium difficile infections. Anaerobe 41, 85-90, doi: 10.1016/j.anaerobe.2016.04.001 (2016).
32 Chien, T. et al. Enhancing the tropism of bacteria via genetically programmed biosensors. Nat Biomed Eng 6, 94-104, doi: 10.1038/s41551-021-00772-3 (2022).
33 Liu, L. et al. An Electrochemical Biosensor with Dual Signal Outputs: Toward Simultaneous Quantification of pH and O(2) in the Brain upon Ischemia and in a Tumor during Cancer Starvation Therapy. Angew Chem Int Ed Engl 56, 10471-10475, doi: 10.1002/anie.201705615 (2017).
34 Corbin, B. D. et al. Metal chelation and inhibition of bacterial growth in tissue abscesses. Science 319, 962-965, doi: 10.1126/science. 1152449 (2008).
35 Perez, J., Springthorpe, V. S. & Sattar, S. A. Clospore: a liquid medium for producing high titers of semi-purified spores of Clostridium difficile. J AOAC Int 94, 618-626 (2011).

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps. Moreover, the scope of the present application is not intended to be limited to the particular embodiments of the process, machine, manufacture, composition of matter, means, methods and steps described in the specification.

What is claimed is:

1. A method of monitoring intestinal inflammation in an individual and/or treating intestinal inflammation in an individual, comprising the steps of:
(a) providing to the individual an effective amount of a population of non-pathogenic bacteria comprising at least two engineered polynucleotides, wherein:
(1) a first said polynucleotide comprises one or more calprotectin-responsive sequences operably linked to expression of one or more proteins having DNA inversion activity; and
(2) a second said polynucleotide comprises a reverse orientation of a gene product of interest flanked by attachment or recognition sites for the one or more proteins having DNA inversion activity, wherein exposure of the one or more proteins having DNA inversion activity to the second polynucleotide results in inversion of the reverse orientation of the gene product into an orientation of the gene product by which a functional gene product is produced; wherein:
(b1) the gene product of interest is a detectable readout product, and the method further comprises examining the feces of said individual for the detectable readout product;
and/or
(b2) the gene product of interest is a therapeutic gene, and the intestinal inflammation is treated.

2. The method of claim 1, wherein in (b2), one or more calprotectin-responsive sequences are operably linked to expression of the therapeutic gene.

3. The method of claim 1 or 2, wherein the first polynucleotide and/or second polynucleotide further comprise expression of the zinc uptake regulator (zur) repressor.

4. The method of claim 3, wherein the expression of zur is regulated by a constitutive promoter.

5. The method of claim 4, wherein the constitutive promoter is J23114, J23109, J23100, J23110, J23119, and/or J23113.

6. The method of any one of claims 1-5, wherein the therapeutic gene is IL-10 or a fusion of IL-10 with a secretion facilitating protein.

7. The method of claim 6, wherein the secretion facilitating protein is YebF or OsmY.

8. The method of any one of claims 1-7, wherein the calprotectin-responsive sequence is a bacterial regulatory sequence.

9. The method of any one of claims 1-8, wherein the calprotectin-responsive sequence comprises a functional part or all of one or more promoters from Escherichia coli Nissle 1917 (EcN).

10. The method of claim 9, wherein the calprotectin-responsive sequence comprises a functional part or all of one or more promoters from Escherichia coli Nissle 1917 (EcN) that results in upregulation of the one or more proteins having DNA inversion activity.

11. The method of any one of claims 1-10, wherein the calprotectin-responsive sequence is directly or indirectly sensitive to a metal to which calprotectin binds.

12. The method of claim 11, wherein the metal is free zinc, iron, manganese, or a combination thereof.

13. The method of any one of claims 1-12, wherein the calprotectin-responsive sequence comprises a functional part or all of alternative ribosomal protein operon (ykgMO), the siderophore enterobactin (ent) operon, and/or an ABC transporter operon (abt).

14. The method of any one of claims 1-13, wherein the readout product is a detectable colorimetric, ultraviolet, ultrasound, and/or fluorescent marker.

15. The method of any one of claims 1-14, wherein the readout product is detectable upon conversion by an enzyme of a substrate to a detectable product.

16. The method of any one of claims 1-15, wherein the readout product is detectable upon conversion by an enzyme of a pro-dye to a visible dye.

17. The method of any one of claims 1-16, wherein the readout product is detectable to the naked eye.

18. The method of any one of claims 1-17, wherein the examining step lacks the need for handling of the feces.

19. The method of any one of claims 1-18, wherein the readout product is one or more of the following: violacein, one or more chromoproteins, one or more carotenoids, one or more phycobilins, one or more anthocyanins, and/or indigo.

20. The method of any one of claims 1-19, wherein the readout product is green fluorescence protein, yellow fluorescent protein, blue fluorescent protein, mCherry, or cyan fluorescent protein.

21. The method of any one of claims 1-20, wherein the providing step occurs orally.

22. The method of any one of claims 1-21, wherein the providing step is performed by the individual.

23. The method of any one of claims 1-22, wherein the providing step is performed once.

24. The method of any one of claims 1-22, wherein the providing step occurs on a regular basis.

25. The method of any one of claim 1-22 or 24, wherein the providing step occurs daily or weekly or monthly.

26. The method of any one of claims 1-25, wherein the providing step occurs during the presence or absence of one or more symptoms of intestinal inflammation.

27. The method of any one of claims 1-26, wherein the examining step of the feces for the detectable readout product is performed by the individual.

28. The method of any one of claims 1-27, wherein when the readout product is detected, the individual obtains treatment for the intestinal inflammation.

29. The method of any one of claims 1-28, wherein the intestinal inflammation is from inflammatory bowel disease (IBD) or from an infection.

30. The method of claim 29, wherein the IBD is Crohn's Disease (CD) or ulcerative colitis (UC).

31. The method of claim 29, wherein the infection is Clostridium difficile.

32. The method of any one of claims 1-31, wherein when the readout product is detected, the individual receives treatment of the inflammation prior to onset of one or more symptoms.

33. The method of any one of claims 1-32, wherein the amount of bacteria is in a range from 10⁶ to 10¹¹.

34. The method of any one of claims 1-33, wherein the bacteria are E. coli, Lactobacillus reuteri, Lactococcus lactis, Bacteroides thetaiotamicro, or Bacillus subtilis.

35. A method of treating intestinal inflammation in an individual, comprising the step of providing to the individual an effective amount of a population of non-pathogenic bacteria comprising one or more engineered polynucleotides, wherein said polynucleotide(s) comprises:
(a) one or more calprotectin-responsive sequences operably linked to expression of a therapeutic gene; and
(b) sequence of the zinc uptake regulator (zur) repressor.

36. The method of claim 35, wherein the sequences of (a) and (b) are on the same polynucleotide.

37. The method of claim 35, wherein the sequences of (a) and (b) are on different polynucleotides.

38. The method of any one of claims 35-37, wherein the expression of zur is regulated by a constitutive promoter.

39. The method of claim 38, wherein the constitutive promoter is J23114 or J23109.

40. The method of any one of claims 35-39, wherein the therapeutic gene is IL-10 or a fusion of IL-10 with a facilitating carrier protein.

41. The method of claim 40, wherein the facilitating carrier protein is YebF.

42. The method of any one of claims 35-41, wherein at substantially the same time as, or prior to, the providing step, the individual is provided an effective amount of a population of non-pathogenic bacteria comprising at least two engineered polynucleotides, wherein:
(1) a first said polynucleotide comprises one or more calprotectin-responsive sequences operably linked to expression of one or more proteins having DNA inversion activity; and
(2) a second said polynucleotide comprises a reverse orientation of a gene product of interest flanked by attachment or recognition sites for the one or more proteins having DNA inversion activity, wherein exposure of the one or more proteins having DNA inversion activity to the second polynucleotide results in inversion of the reverse orientation of the gene product into an orientation of the gene product by which a functional gene product is produced;
wherein the gene product of interest is a detectable readout product, and the method further comprises examining the feces of said individual for the detectable readout product.

43. The method of claim 42, wherein when both providing steps occur at substantially the same time, the non-pathogenic bacteria of each providing step is the same bacteria.

44. A non-pathogenic bacterial composition, comprising at least two engineered polynucleotides, wherein:
(1) a first said polynucleotide comprises one or more calprotectin-responsive sequences operably linked to expression of an integrase; and
(2) a second said polynucleotide comprises a reverse orientation of a gene product of interest flanked by attachment sites for the integrase; wherein:
(a) the gene product of interest is a detectable readout product; and/or
(b) the gene product of interest is a therapeutic gene.

45. The composition of claim 44, wherein in (2), one or more calprotectin-responsive sequences are operably linked to expression of the gene product of interest.

46. The composition of claim 44 or 45, wherein the polynucleotide further comprises expression of the zinc uptake regulator (zur) repressor.

47. The composition of claim 46, wherein the expression of zur is regulated by a constitutive promoter.

48. The composition of claim 47, wherein the constitutive promoter is J23114 or J23109.

49. The composition of any one of claims 44-48, wherein the therapeutic gene is IL-10, Elafin, IL-22, IL-36, anti-TNF nanobodies or a fusion thereof with a facilitating carrier protein.

50. The composition of claim 49, wherein the facilitating carrier protein is YebF.

51. The composition of any one of claims 44-50, wherein the calprotectin-responsive sequence is a bacterial regulatory sequence.

52. The composition of any one of claims 44-51, wherein the calprotectin-responsive sequence comprises a functional part or all of one or more promoters from Escherichia coli Nissle 1917 (EcN).

53. The composition of claim 52, wherein the calprotectin-responsive sequence comprises a functional part or all of one or more promoters from Escherichia coli Nissle 1917 (EcN) that results in upregulation of the integrase.

54. The composition of any one of claims 44-53, wherein the calprotectin-responsive sequence is directly or indirectly sensitive to a metal to which calprotectin binds.

55. The composition of claim 54, wherein the metal is free zinc, iron, manganese, or a combination thereof.

56. The composition of any one of claims 44-55, wherein the readout product is a detectable colorimetric, ultraviolet, ultrasound, and/or fluorescent marker.

57. The composition of any one of claims 44-56, wherein the readout product is one or more of the following: violacein, one or more carotenoids, one or more phycobilins, one or more anthocyanins, and indigo.

58. The composition of any one of claims 44-57, wherein the readout product is green fluorescence protein, yellow fluorescent protein, blue fluorescent protein, one or more phycobilins, one or more anthocyanins, or cyan fluorescent protein.

59. A system, comprising the composition of any one of claims 44-58.

60. The system of claim 59, further comprising a storage device, a detectable readout analysis device, or both.

61. A kit comprising the composition of any one of claims 44-58, or the system of claim 59 or 60.


SEQUENCE LISTING (19 sequences)

SEQ ID NO: 1 - DNA, 364 bp, Escherichia coli
  source 1..364: mol_type = genomic DNA
taacggcaataaactgttcacttcagtgatatttaaaatatgcatcctctcccttttttgtaagtaattattatatccgtgggagaggaatacacattgtcaggtaatcaatcatgctgcaataaatcatcggccagtaaagtggagatagcctccattctcgaaaaatccatactctcagcgaaaccatcatcaatcactcatccaggcgtttatgggagcgtcgccaatggctgctaacaatgccagacttccccgttgcggaaattccacatcccacaaatagtcacagtgattgggtgttgaaatgatccggatgagcatgtatctttacggttatgttataacataacaggtaaaaatg

SEQ ID NO: 2 - DNA, 336 bp, Escherichia coli
  source 1..336: mol_type = genomic DNA
aacagacccgaagaaaatgaaatataagaaaagatcaacgagtgaagaaaaagttcaaaaaatggctgccggggaggaaggaaagtaccggatggaaagagtcccccctaaagcagactgacagacataacaaatccccggggggatttgtgtataagagacagtacttatctggaggtaattgcaatatctctgtgaacttacacagggtgggcttaccgcatacactgacacttagcggatcgacagaacattattaacagagcatcactgaacgctacataatcagagttgcataaataaaatgttattaacatcacaatcacaacatttcga

SEQ ID NO: 3 - DNA, 187 bp, Escherichia coli
  source 1..187: mol_type = genomic DNA
aagtcagatcctgttattaatgaagttaatgcttctcattttcattgtaaccacaaccagatgcaaccccgagttgcagattgcgttacctcaagagttgacatagtgcgcgtttgcttttaggttagcgaccgaaaatataaatgataatcattattaaagcctttatcattttgtggaggatgat

SEQ ID NO: 4 - DNA, 35 bp, synthetic construct
  source 1..35: mol_type = other DNA
tttatggctagctcagtcctaggtacaatgctagc

SEQ ID NO: 5 - DNA, 35 bp, synthetic construct
  source 1..35: mol_type = other DNA
tttacagctagctcagtcctagggactgtgctagc

SEQ ID NO: 6 - DNA, 35 bp, synthetic construct
  source 1..35: mol_type = other DNA
ttgacagctagctcagtcctaggtataatgctagc

SEQ ID NO: 7 - DNA, 120 bp, Bacillus subtilis
  source 1..120: mol_type = genomic DNA
atgccatgaggtttacgatgtgaaacagagggaaggatataagaagattgacaaaacgtttgtaaggttataaaataaaacgtaattattacgatttattgaaaggagatacccatgaaa

SEQ ID NO: 8 - DNA, 110 bp, Bacillus subtilis
  source 1..110: mol_type = genomic DNA
acaaagcggtatgcttgcttttttgaatggtgaatgatacattttaaatcgtaacaatttcgatttaaagggaggctatgtgacttggctaaatcgtaatattacgattt

SEQ ID NO: 9 - DNA, 3059 bp, synthetic construct
  source 1..3059: mol_type = other DNA
gctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagtaacggcaataaactgttcacttcagtgatatttaaaatatgcatcctctcccttttttgtaagtaattattatatccgtgggagaggaatacacattgtcaggtaatcaatcatgctgcaataaatcatcggccagtaaagtggagatagcctccattctcgaaaaatccatactctcagcgaaaccatcatcaatcactcatccaggcgtttatgggagcgtcgccaatggctgctaacaatgccagacttccccgttgcggaaattccacatcccacaaatagtcacagtgattgggtgttgaaatgatccggatgagcatgtatctttacggttatgttataacataacaggtaaaaatgaaagaggagaaatactagatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgatgataataatctagaccaggcatcaaataaaacgaaaggctcagtcgaaagactgggcctttcgttttatctgttgtttgtcggtgaacgctctctactagagtcacactggctcaccttcgggtgggcctttctgcgtttataggtacccgtggatcctctagagtcgaccgagcggtatcagctcactcaaaggcggtaatacggttatccacagaatcaggggataacgcaggaaagaacatgtgagcaaaaggccagcaaaaggccaggaaccgtaaaaaggccgcgtt

SEQ ID NO: 10 - DNA, 2882 bp, synthetic construct
  source 1..2882: mol_type = other DNA
gctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaagtcagatcctgttattaatgaagttaatgcttctcattttcattgtaaccacaaccagatgcaaccccgagttgcagattgcgttacctcaagagttgacatagtgcgcgtttgcttttaggttagcgaccgaaaatataaatgataatcattattaaagcctttatcattttgtggaggatgataaagaggagaaatactagatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgatgataataatctagaccaggcatcaaataaaacgaaaggctcagtcgaaagactgggcctttcgttttatctgttgtttgtcggtgaacgctctctactagagtcacactggctcaccttcgggtgggcctttctgcgtttataggtacccgtggatcctctagagtcgaccgagcggtatcagctcactcaaaggcggtaatacggttatccacagaatcaggggataacgcaggaaagaacatgtgagcaaaaggccagcaaaaggccaggaaccgtaaaaaggccgcgtt

SEQ ID NO: 11 - DNA, 3031 bp, synthetic construct
  source 1..3031: mol_type = other DNA
gctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaacagacccgaagaaaatgaaatataagaaaagatcaacgagtgaagaaaaagttcaaaaaatggctgccggggaggaaggaaagtaccggatggaaagagtcccccctaaagcagactgacagacataacaaatccccggggggatttgtgtataagagacagtacttatctggaggtaattgcaatatctctgtgaacttacacagggtgggcttaccgcatacactgacacttagcggatcgacagaacattattaacagagcatcactgaacgctacataatcagagttgcataaataaaatgttattaacatcacaatcacaacatttcgaaaagaggagaaatactagatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgatgataataatctagaccaggcatcaaataaaacgaaaggctcagtcgaaagactgggcctttcgttttatctgttgtttgtcggtgaacgctctctactagagtcacactggctcaccttcgggtgggcctttctgcgtttataggtacccgtggatcctctagagtcgaccgagcggtatcagctcactcaaaggcggtaatacggttatccacagaatcaggggataacgcaggaaagaacatgtgagcaaaaggccagcaaaaggccaggaaccgtaaaaaggccgcgtt

SEQ ID NO: 12 - DNA, 3689 bp, synthetic construct
  source 1..3689: mol_type = other DNA
cgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcggtcgactctagaggatccacgggtacctaacggcaataaactgttcacttcagtgatatttaaaatatgcatcctctcccttttttgtaagtaattattatatccgtgggagaggaatacacattgtcaggtaatcaatcatgctgcaataaatcatcggccagtaaagtggagatagcctccattctcgaaaaatccatactctcagcgaaaccatcatcaatcactcatccaggcgtttatgggagcgtcgccaatggctgctaacaatgccagacttccccgttgcggaaattccacatcccacaaatagtcacagtgattgggtgttgaaatgatccggatgagcatgtatctttacggttatgttataacataacaggtaaaaatgaaagaggagaaatactagatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgatgataataatctagaccaggcatcaaataaaacgaaaggctcagtcgaaagactgggcctttcgttttatctgttgtttgtcggtgaacgctctctactagagtcacactggctcaccttcgggtgggcctttctgcgtttatactcgaggtgaagacgaaagggcctcgtgatacgcctatttttataggttaatgtcatgataataatggtttcttagacgagcgttttgttatcaataaaaaaggccccccgatttgggaggcctttttagttagaaagctttagcgaggtttctttttcacctgaatagagtggtcatgatggcactgttcaggatgacgacacgcttctacttctacacatgctgaacataatccatgtgcttcaataacattatgccgcagggcaaaacccatttttgccgccagcgtatgcatgatgtcttccacgccttcagcacattcttctttcaccgcgccgcagcgatcgcaaataaacatggctgaagtatgggtgggctgatcgaacagatgacagagcacataactgttggtggattccaccttatgcacaaaaccttgttcaagcagaaactccagcgcgcgataaaccgttggcggcttggcttgcggttcagcttcgcgcagcaaatcaagcagatcataagcgctgatagcgccatcctgcagactcatcaggcgcaacacttcgaggcgctgtggggtcaggcgcacattacgctgtgcgcagattttttcagcctgcgctaataactcctgcgttgtggtcttttccatatattatctcctgaattcgctagcacagtccctaggactgagctagctgtaaatcgatatctggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttgatatcgagctcgcttggactcctgttgatagatccagtaatgacctcagaactccatctggatttgttcagaacgctcggttgccgccgggcgttttttattggtgagaatccaagcactagtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggt

SEQ ID NO: 13 - DNA, 4398 bp, synthetic construct
  source 1..4398: mol_type = other DNA
cgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcggtcgactctagaggatccacgggtacctaacggcaataaactgttcacttcagtgatatttaaaatatgcatcctctcccttttttgtaagtaattattatatccgtgggagaggaatacacattgtcaggtaatcaatcatgctgcaataaatcatcggccagtaaagtggagatagcctccattctcgaaaaatccatactctcagcgaaaccatcatcaatcactcatccaggcgtttatgggagcgtcgccaatggctgctaacaatgccagacttccccgttgcggaaattccacatcccacaaatagtcacagtgattgggtgttgaaatgatccggatgagcatgtatctttacggttatgttataacataacaggtaaaggctagcaaaacaataactaggattcgaatgaaagttgccgtttattgtcgtgttagcaccctggaacagaaagaacatggtcatagcattgaagaacaagagcgtaaactgaaaagcttctgcgatattaatgattggaccgtgtatgatacctatatcgatgcaggttatagcggtgcaaaacgtgatcgtccggaactgcagcgtctgatgaatgatattaacaaatttgatctggtgctggtgtataaactggatcgtctgacccgtaatgttcgtgatctgctggacctgctggaaatctttgaaaaaaatgatgtgagctttcgtagcgccaccgaagtttatgataccaccaccgcaatgggtcgtctgtttgttaccctggttggtgcaatggcagaatgggaacgtgaaaccattcgtgaacgtacccagatgggtaaactggcagcactgcgtaaaggtattatgctgaccacccctccgttttattatgaccgtgtggataataagtttgtgccgaacaaatacaaagacgttattctgtgggcatatgacgaagcaatgaaaggtcagagcgcaaaagcaattgcacgcaaactgaataatagcgatattccgcctccgaataatacccagtggcagggtcgtaccattacccatgccctgcgtaatccgtttacccgtggtcattttgattggggtggtgtgcatattgaaaataaccatgaaccgatcatcaccgatgagatgtatgagaaagttaaagatcgcctgaatgaacgcgtgaacaccaaaaaagttcgtcataccagcatttttcgtggcaaactggtttgtccggtttgtaatgcacgcctgaccctgaatagccataaaaagaaaagcaatagcggctatatctttgtgaaacagtactactgcaacaactgtaaagttaccccgaatctgaaaccggtgtacatcaaagaaaaagaagtgattaaagttttttacaattatctgaaacgcttcgatctggaaaaatatgaggttacccagaaacagaacgaaccggaaatcaccatcgatatcaataaagttatggaacagcgcaaacgctaccataaactgtatgcaagcggtctgatgcaagaagatgaactgtttgacctgattaaagaaaccgatcagaccattgccgaatatgaaaaacagaatgaaaaccgcgaagtgaagcagtatgatatcgaagatatcaaacagtataaagatctgctgttagaaatgtgggatatcagctccgatgaagataaagaggactttatcaaaatggcgattaaaaacatctattttgaatatatcattggcaccggtaacaccagccgtaaacgtaatagcctgaaaattacgagcattgaattctattaacagataaaaaaaatccttagctttcgctaaggatgattaataatctagaccaggcatcaaataaaacgaaaggctcagtcgaaagactgggcctttcgttttatctgttgtttgtcggtgaacgctctctactagagtcacactggctcaccttcgggtgggcctttctgcgtttatactcgaggtgaagacgaaagggcctcgtgatacgcctatttttataggttaatgtcatgataataatggtttcttagacgagcgttttgttatcaataaaaaaggccccccgatttgggaggcctttttagttagaaagctttagcgaggtttctttttcacctgaatagagtggtcatgatggcactgttcaggatgacgacacgcttctacttctacacatgctgaacataatccatgtgcttcaataacattatgccgcagggcaaaacccatttttgccgccagcgtatgcatgatgtcttccacgccttcagcacattcttctttcaccgcgccgcagcgatcgcaaataaacatggctgaagtatgggtgggctgatcgaacagatgacagagcacataactgttggtggattccaccttatgcacaaaaccttgttcaagcagaaactccagcgcgcgataaaccgttggcggcttggcttgcggttcagcttcgcgcagcaaatcaagcagatcataagcgctgatagcgccatcctgcagactcatcaggcgcaacacttcgaggcgctgtggggtcaggcgcacattacgctgtgcgcagattttttcagcctgcgctaataactcctgcgttgtggtcttttccatatattatctcctgaattcgctagcacagtccctaggactgagctagctgtaaatcgatatctggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttgatatcgagctcgcttggactcctgttgatagatccagtaatgacctcagaactccatctggatttgttcagaacgctcggttgccgccgggcgttttttattggtgagaatccaagcactagtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggt

SEQ ID NO: 14 - DNA, 3393 bp, synthetic construct
  source 1..3393: mol_type = other DNA
aggtagagacccacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaagtatatatgagtaaacttggtctgacagctcgagcttacccgtcttactgtccctagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgtgatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaatcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgttacgtgcccgatcaactgcgtttatatactagtagcggcagctgcagtccggcaaaaaagggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatttgacagctagctcagtcctaggtataatgctagcagttcgatgagagcgataaccaatcatcagataactatggcggcacgtgcattaaccacggttgtatcccgtctaaagtactcgttcatcatttgtacagttcatccataccatgcgtgatgcccgctgcggttacgaactccagcagaaccatatgatcgcgtttctcgttcggatctttagacagaacgctttgcgtgctcagatagtgattgtctggcagcagaacaggaccatcaccgattggagtgttttgctggtagtgatcagccagctgcacgctgccatcctccacgttgtggcgaattttaaaattcgctttaatgccatttttttgtttatcggcggtgatgtaaacattgtggctgttaaaattgtattccagcttatggcccaggatattgccgtcttctttaaagtcaatgcctttcagctcaatgcggtttaccagggtatcgccttcaaatttcacttccgcacgcgttttgtacgtgccgtcatccttaaaggaaatcgtgcgttcctgcacatagccttccggcatggcggacttgaagaagtcatgctgcttcatatggtccggataacgagcaaagcactgaacaccataagtcagcgtcgttaccagagtcggccaaggtaccggcagtttaccagtagtacagatgaacttcagcgtcagtttaccattagttgcgtcaccttcaccctcgccacgcacggaaaacttatgaccgttgacatcaccatccagttccaccagaatagggacgacaccagtgaacagctcttcgcctttacgcatctagtatttctcctctttctctagattaaacaaaattatttgtagaggctgtttcgtcctcacggactcatcagaccggaaagcacatccggtgacagctattttattatggaagtttgttcactcaacattgcaagactgtacatacttccatagtttattaatataaacgcagaaaggcccacccgaaggtgagccagtgtgactctagtagagagcgttcaccgacaaacaacagataaaacgaaaggcccagtctttcgactgagcctttcgttttatttgatgcctggctagggcccatacccaaaaaaaaaccccgcccctgacagggcggggtttttttt

SEQ ID NO: 15 - DNA, 4044 bp, synthetic construct
  source 1..4044: mol_type = other DNA
aggtagagacccacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaagtatatatgagtaaacttggtctgacagctcgagcttacccgtcttactgtccctagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgtgatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaatcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgttacgtgcccgatcaactgcgtttatatactagtagcggcagctgcagtccggcaaaaaagggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatttgacagctagctcagtcctaggtataatgctagcagttcgatgagagcgataaccaatcatcagataactatggcggcacgtgcattaaccacggttgtatcccgtctaaagtactcgtttaggcctcttttgccaccgggatttcggacttgtcgctcaggtaagcctccaggctgtcgtccagagcatcgatccacggggtatggtgctttggagccatcgtgccggtcatcagggagcgatagctgtggtcgcgaaacgtcatgatgttctctttcttatgatgtttccattccaggaacgttttgttcgtcgccggaatatcaaagctcggataatcggtcatgtcgatcaggttctggatatagtcaccctggtaggtatacatctcttcagccgtgaccagcgtcagctctttctcgcgccacgccatgctgtcggccttcatttcttctttgctcggcaacggcaaacggcccatgatcacatcacgcgcataccaagcttgggcatcgaacatgttgaagctgtaccattggtcttgcataccaatgtagaagaacttcggattatcctcccaaacaacgcccttgtacaggttcagcggccacaggcgattgttggtcaccaggcgcaagtcgtcattcagaaacgggaagtggtggatgtaaccggtgcacaggatgatggcatcaactttctccgaagaaccatctgcgaagtaagcattctcggtatccacacgcaccaggttcggacgctcgtcccagttctccggccatttgtaacccatcggtgcggtgcgatagcagctaatcagtttcttcgcgccatacttgtagcactggctgccaatatcttccgcagagtacgagctgcccaccaacagaacggtcttatccttgaactccagcgcatcacgaaagtcgtgggcatgcagaatacgaccaccgaacttttcgaagccttcgaactccgggacatacggggtgctgaagtgaccggtacagcaaaccacatagtcaaattcttcgctatagatggtgtcagtggtatggtcctgaaccgtcacagtaaaggtctgagaatcctcattgaactcaacatgacgaaccgcggtattgaaacggatgtatttgcggacacccgccttttccacgcgacccttgatatagtcccacagcacttcacgcggtgggtagctggcaattggcttaccaaagtgttcatcaaaggtgtaatcagcaaactccaaacattctttcggaccgttggaccacaggtaacggtacatggagctgtggaccggttcgccgttttcgtccagaccggtacgccaagtatagttccactggccaccccagtccgcctgtttctcaaaacaaaccaactccgggatctcagcgcctttctcttgtgcgctttgaaacgcacgcaattgcgccatacccgatgggcctgcgcccagaattgcaatacgggtcgccattttttacctcctaaaagtctctagattaaacaaaattatttgtagaggctgtttcgtcctcacggactcatcagaccggaaagcacatccggtgacagctattttattatggaagtttgttcactcaacattgcaagactgtacatacttccatagtttattaatataaacgcagaaaggcccacccgaaggtgagccagtgtgactctagtagagagcgttcaccgacaaacaacagataaaacgaaaggcccagtctttcgactgagcctttcgttttatttgatgcctggctagggcccatacccaaaaaaaaaccccgcccctgacagggcggggtttttttt

SEQ ID NO: 16 - DNA, 3379 bp, synthetic construct
  source 1..3379: mol_type = other DNA
cgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcggtcgactctagaggatccacgggtacctaacggcaataaactgttcacttcagtgatatttaaaatatgcatcctctcccttttttgtaagtaattattatatccgtgggagaggaatacacattgtcaggtaatcaatcatgctgcaataaatcatcggccagtaaagtggagatagcctccattctcgaaaaatccatactctcagcgaaaccatcatcaatcactcatccaggcgtttatgggagcgtcgccaatggctgctaacaatgccagacttccccgttgcggaaattccacatcccacaaatagtcacagtgattgggtgttgaaatgatccggatgagcatgtatctttacggttatgttataacataacaggtaaaggctagcaaaacaataactaggattcgaggagaaaaacatgaaaaaaagaggggcgtttttagggctgttgttggtttctgcctgcgcatcagttttcgctgccaataatgaaaccagcaagtcggtcactttcccaaagtgtgaaggtctggatgctgccggaattgccgcgagcgtaaaacgtgattaccaacaaaatcgcgtggcgcgctgggctgatgatcaaaaaattgtcggtcaggccgatcccgtggcatgggtcagtttgcaggacattcaggggaaagatgataaatggtcagtaccgctaaccgtgcgtggtaaaagtgccgatattcattaccaggtcagcgtggactgcaaagcgggaatggcggaatatcagcggcgttaacagataaaaaaaatccttagctttcgctaaggatgattaataatctagaccaggcatcaaataaaacgaaaggctcagtcgaaagactgggcctttcgttttatctgttgtttgtcggtgaacgctctctactagagtcacactggctcaccttcgggtgggcctttctgcgtttatactcgaggtgaagacgaaagggcctcgtgatacgcctatttttataggttaatgtcatgataataatggtttcttagacgagcgttttgttatcaataaaaaaggccccccgatttgggaggcctttttagttagaaagctttagcgaggtttctttttcacctgaatagagtggtcatgatggcactgttcaggatgacgacacgcttctacttctacacatgctgaacataatccatgtgcttcaataacattatgccgcagggcaaaacccatttttgccgccagcgtatgcatgatgtcttccacgccttcagcacattcttctttcaccgcgccgcagcgatcgcaaataaacatggctgaagtatgggtgggctgatcgaacagatgacagagcacataactgttggtggattccaccttatgcacaaaaccttgttcaagcagaaactccagcgcgcgataaaccgttggcggcttggcttgcggttcagcttcgcgcagcaaatcaagcagatcataagcgctgatagcgccatcctgcagactcatcaggcgcaacacttcgaggcgctgtggggtcaggcgcacattacgctgtgcgcagattttttcagcctgcgctaataactcctgcgttgtggtcttttccatatattatctcctgaattcgctagcacagtccctaggactgagctagctgtaaatcgatatctggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttgatatcgagctcgcttggactcctgttgatagatccagtaatgacctcagaactccatctggatttgttcagaacgctcggttgccgccgggcgttttttattggtgagaatccaagcactagtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggt

SEQ ID NO: 17 - DNA, 3859 bp, synthetic construct
  source 1..3859: mol_type = other DNA
cgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcggtcgactctagaggatccacgggtacctaacggcaataaactgttcacttcagtgatatttaaaatatgcatcctctcccttttttgtaagtaattattatatccgtgggagaggaatacacattgtcaggtaatcaatcatgctgcaataaatcatcggccagtaaagtggagatagcctccattctcgaaaaatccatactctcagcgaaaccatcatcaatcactcatccaggcgtttatgggagcgtcgccaatggctgctaacaatgccagacttccccgttgcggaaattccacatcccacaaatagtcacagtgattgggtgttgaaatgatccggatgagcatgtatctttacggttatgttataacataacaggtaaaggctagcaaaacaataactaggattcgaggagaaaaacatgaaaaaaagaggggcgtttttagggctgttgttggtttctgcctgcgcatcagttttcgctgccaataatgaaaccagcaagtcggtcactttcccaaagtgtgaaggtctggatgctgccggaattgccgcgagcgtaaaacgtgattaccaacaaaatcgcgtggcgcgctgggctgatgatcaaaaaattgtcggtcaggccgatcccgtggcatgggtcagtttgcaggacattcaggggaaagatgataaatggtcagtaccgctaaccgtgcgtggtaaaagtgccgatattcattaccaggtcagcgtggactgcaaagcgggaatggcggaatatcagcggcgtagcccgggccaaggcacgcagagcgaaaacagctgcacccattttccgggcaacctgccgaacatgctgcgcgatctgcgcgatgcgtttagccgcgtgaaaaccttttttcagatgaaagatcagctggataacctgctgctgaaagaaagcctgctggaagattttaaaggctatctgggctgccaagcgctgagcgaaatgattcagttttatctggaagaagtgatgccgcaagcggaaaaccaagatccggatattaaagcgcatgtgaacagcctgggcgaaaacctgaaaaccctgcgcctgcgcctgcgccgctgccatcgctttctgccgtgcgaaaacaaaagcaaagcggtggaacaagtgaaaaacgcgtttaacaaactgcaagaaaagggaatatataaggcaatgtcagaattcgacatctttattaactatattgaagcgtatatgaccatgaaaattcgcaactaacagataaaaaaaatccttagctttcgctaaggatgattaataatctagaccaggcatcaaataaaacgaaaggctcagtcgaaagactgggcctttcgttttatctgttgtttgtcggtgaacgctctctactagagtcacactggctcaccttcgggtgggcctttctgcgtttatactcgaggtgaagacgaaagggcctcgtgatacgcctatttttataggttaatgtcatgataataatggtttcttagacgagcgttttgttatcaataaaaaaggccccccgatttgggaggcctttttagttagaaagctttagcgaggtttctttttcacctgaatagagtggtcatgatggcactgttcaggatgacgacacgcttctacttctacacatgctgaacataatccatgtgcttcaataacattatgccgcagggcaaaacccatttttgccgccagcgtatgcatgatgtcttccacgccttcagcacattcttctttcaccgcgccgcagcgatcgcaaataaacatggctgaagtatgggtgggctgatcgaacagatgacagagcacataactgttggtggattccaccttatgcacaaaaccttgttcaagcagaaactccagcgcgcgataaaccgttggcggcttggcttgcggttcagcttcgcgcagcaaatcaagcagatcataagcgctgatagcgccatcctgcagactcatcaggcgcaacacttcgaggcgctgtggggtcaggcgcacattacgctgtgcgcagattttttcagcctgcgctaataactcctgcgttgtggtcttttccatatattatctcctgaattcgctagcacagtccctaggactgagctagctgtaaatcgatatctggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttgatatcgagctcgcttggactcctgttgatagatccagtaatgacctcagaactccatctggatttgttcagaacgctcggttgccgccgggcgttttttattggtgagaatccaagcactagtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggt

SEQ ID NO: 18 - DNA, 5204 bp, synthetic construct
  source 1..5204: mol_type = other DNA
cgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcggtcgactctagaggatccacgggtacctaacggcaataaactgttcacttcagtgatatttaaaatatgcatcctctcccttttttgtaagtaattattatatccgtgggagaggaatacacattgtcaggtaatcaatcatgctgcaataaatcatcggccagtaaagtggagatagcctccattctcgaaaaatccatactctcagcgaaaccatcatcaatcactcatccaggcgtttatgggagcgtcgccaatggctgctaacaatgccagacttccccgttgcggaaattccacatcccacaaatagtcacagtgattgggtgttgaaatgatccggatgagcatgtatctttacggttatgttataacataacaggtaaaggctagcaaaacaataactaggattcgaggagaaaaacatgaaaaaaagaggggcgtttttagggctgttgttggtttctgcctgcgcatcagttttcgctgccaataatgaaaccagcaagtcggtcactttcccaaagtgtgaaggtctggatgctgccggaattgccgcgagcgtaaaacgtgattaccaacaaaatcgcgtggcgcgctgggctgatgatcaaaaaattgtcggtcaggccgatcccgtggcatgggtcagtttgcaggacattcaggggaaagatgataaatggtcagtaccgctaaccgtgcgtggtaaaagtgccgatattcattaccaggtcagcgtggactgcaaagcgggaatggcggaatatcagcggcgtagcccgggccaaggcacgcagagcgaaaacagctgcacccattttccgggcaacctgccgaacatgctgcgcgatctgcgcgatgcgtttagccgcgtgaaaaccttttttcagatgaaagatcagctggataacctgctgctgaaagaaagcctgctggaagattttaaaggctatctgggctgccaagcgctgagcgaaatgattcagttttatctggaagaagtgatgccgcaagcggaaaaccaagatccggatattaaagcgcatgtgaacagcctgggcgaaaacctgaaaaccctgcgcctgcgcctgcgccgctgccatcgctttctgccgtgcgaaaacaaaagcaaagcggtggaacaagtgaaaaacgcgtttaacaaactgcaagaaaagggaatatataaggcaatgtcagaattcgacatctttattaactatattgaagcgtatatgaccatgaaaattcgcaactaaggatccataatcaggatcaataaaactgttgcagaaatgatttcattcataactcaaattccctgataattgccgcggactttctgcgtgctaacaaagcaggataagtcgcattactgatggcttcgctatcattgattaatttcacttgcgactttggctgctttttgtatggtgaaagatgtgccaggaggagaccggcacatttatacagcacacatctttgcaggaaaaaacgcttatgaaaaatgttggttttatcggctggcgcggtatggtcggctccgttctcatgcaacgcatggttgaagagcgcgacttcgacgccattcgccctgtcttcttttctacttctcagcttggtcaggctgcgccgtcttttggcggaaccactggcacacttcaggatgcctttgatctggaggcgctaaaggccctcgatatcattgtgacctgtcagggcggcgattataccaacgaaatctatccaaagcttcgtgaaagcggatggcaaggttactggattgacgcagcatcatctctgcgcatgaaagatgacgccatcatcattcttgaccccgtcaatcaggacgtcattaccgacggattaaataatggcatcaggacttttgttggcggtaactgtaccgtaagcctgatgttgatgtcgctgggtggtttattcgccaatgatcttgttgattgggtgtccgttgcaacctaccaggccgcttccggcggtggtgcgcgacatatgcgtgagttattaacccaaatgggccatctgtatggccatgtggcagatgaactcgcgaacccgtcctctgctattctcgatatcgaacgcaaagtcacaaccttaacccgtagcggtgagctgccggtagataactttggcgtgccgctggcgggtagcctgattccgtggatcgacaaacagcttgataacggtcagagccgcgaagagtggaaagggcaggcggaaaccaacaagatcctcaacacatcttccgtaattccggtagatggtttatgtgtgcgtgtcggggcattgcgctgccacagccaggcattcactattaaattgaaaaaagatgtgtctattccgaccgtggaagaactgctggctgcgcacaatccgtgggcgaaagttgttccaaacgatcgggaaatcactatgcgtgagctaaccccagctgccgttaccggcacgctgaccacgccggtaggccgtctgcgtaagctgaatatgggaccagagttcctgtcagcctttaccgtgggcgaccagctgctgtggggggccgcggagccgctgcgtcggatgcttcgtcaactggcgtaacagataaaaaaaatccttagctttcgctaaggatgattaataatctagaccaggcatcaaataaaacgaaaggctcagtcgaaagactgggcctttcgttttatctgttgtttgtcggtgaacgctctctactagagtcacactggctcaccttcgggtgggcctttctgcgtttatactcgaggtgaagacgaaagggcctcgtgatacgcctatttttataggttaatgtcatgataataatggtttcttagacgagcgttttgttatcaataaaaaaggccccccgatttgggaggcctttttagttagaaagctttagcgaggtttctttttcacctgaatagagtggtcatgatggcactgttcaggatgacgacacgcttctacttctacacatgctgaacataatccatgtgcttcaataacattatgccgcagggcaaaacccatttttgccgccagcgtatgcatgatgtcttccacgccttcagcacattcttctttcaccgcgccgcagcgatcgcaaataaacatggctgaagtatgggtgggctgatcgaacagatgacagagcacataactgttggtggattccaccttatgcacaaaaccttgttcaagcagaaactccagcgcgcgataaaccgttggcggcttggcttgcggttcagcttcgcgcagcaaatcaagcagatcataagcgctgatagcgccatcctgcagactcatcaggcgcaacacttcgaggcgctgtggggtcaggcgcacattacgctgtgcgcagattttttcagcctgcgctaataactcctgcgttgtggtcttttccatatattatctcctgaattcgctagcacagtccctaggactgagctagctgtaaatcgatatctggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttgatatcgagctcgcttggactcctgttgatagatccagtaatgacctcagaactccatctggatttgttcagaacgctcggttgccgccgggcgttttttattggtgagaatccaagcactagtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggt

SEQ ID NO: 19 - DNA, 4726 bp, synthetic construct
  source 1..4726: mol_type = other DNA
tggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaagtatatatgagtaaacttggtctgacagctcgagcttacccgtcttactgtccctagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgtgatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaatcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgttacgtgcccgatcaactgcgtttatatactagtagcggcagctgcagtccggcaaaaaagggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatttgacagctagctcagtcctaggtataatgctagcagttcgatgagagcgataaccaatcatcagataactatggcggcacgtgcattaaccacggttgtatcccgtctaaagtactcgtctttagttgcgaattttcatggtcatatacgcttcaatatagttaataaagatgtcgaattctgacattgccttatatattcccttttcttgcagtttgttaaacgcgtttttcacttgttccaccgctttgcttttgttttcgcacggcagaaagcgatggcagcggcgcaggcgcaggcgcagggttttcaggttttcgcccaggctgttcacatgcgctttaatatccggatcttggttttccgcttgcggcatcacttcttccagataaaactgaatcatttcgctcagcgcttggcagcccagatagcctttaaaatcttccagcaggctttctttcagcagcaggttatccagctgatctttcatctgaaaaaaggttttcacgcggctaaacgcatcgcgcagatcgcgcagcatgttcggcaggttgcccggaaaatgggtgcagctgttttcgctctgcgtgccttggcccgggctacgccgctgatattccgccattcccgctttgcagtccacgctgacctggtaatgaatatcggcacttttaccacgcacggttagcggtactgaccatttatcatctttcccctgaatgtcctgcaaactgacccatgccacgggatcggcctgaccgacaattttttgatcatcagcccagcgcgccacgcgattttgttggtaatcacgttttacgctcgcggcaattccggcagcatccagaccttcacactttgggaaagtgaccgacttgctggtttcattattggcagcgaaaactgatgcgcaggcagaaaccaacaacagccctaaaaacgcccctctttttttcatgtttttctcctcgaatcctagttattgttttctattttattatggaagtttgttcactcaacattgcaagactgtacatacttccatagtttattaatataaacgcagaaaggcccacccgaaggtgagccagtgtgactctagtagagagcgttcaccgacaaacaacagataaaacgaaaggcccagtctttcgactgagcctttcgttttatttgatgcctggctagggcctacgcccggtagtgatcttatttcattatggtgaaagttggaacctcttacgggtagagacccacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcattacgccagttgacgaagcatccgacgcagcggctccgcggccccccacagcagctggtcgcccacggtaaaggctgacaggaactctggtcccatattcagcttacgcagacggcctaccggcgtggtcagcgtgccggtaacggcagctggggttagctcacgcatagtgatttcccgatcgtttggaacaactttcgcccacggattgtgcgcagccagcagttcttccacggtcggaatagacacatcttttttcaatttaatagtgaatgcctggctgtggcagcgcaatgccccgacacgcacacataaaccatctaccggaattacggaagatgtgttgaggatcttgttggtttccgcctgccctttccactcttcgcggctctgaccgttatcaagctgtttgtcgatccacggaatcaggctacccgccagcggcacgccaaagttatctaccggcagctcaccgctacgggttaaggttgtgactttgcgttcgatatcgagaatagcagaggacgggttcgcgagttcatctgccacatggccatacagatggcccatttgggttaataactcacgcatatgtcgcgcaccaccgccggaagcggcctggtaggttgcaacggacacccaatcaacaagatcattggcgaataaaccacccagcgacatcaacatcaggcttacggtacagttaccgccaacaaaagtcctgatgccattatttaatccgtcggtaatgacgtcctgattgacggggtcaagaatgatgatggcgtcatctttcatgcgcagagatgatgctgcgtcaatccagtaaccttgccatccgctttcacgaagctttggatagatttcgttggtataatcgccgccctgacaggtcacaatgatatcgagggcctttagcgcctccagatcaaaggcatcctgaagtgtgccagtggttccgccaaaagacggcgcagcctgaccaagctgagaagtagaaaagaagacagggcgaatggcgtcgaagtcgcgctcttcaaccatgcgttgcatgagaacggagccgaccataccgcgccagccgataaaaccaacatttttcataagcgttttttcctgcaaagatgtgtgctgtataaatgtgccggtctcctcctggcacatctttcaccatacaaaaagcagccaaagtcgcaagtgaaattaatcaatgatagcgaagccatcagtaatgcgacttatcctgctttgttagcacgcagaaagtccgcggcaattatcagggaatttgagttatgaatgaaatcatttctgcaacagttttattgatcctgattatggatcc